(19) United States
(12) Patent Application Publication
(10) Pub. No.: US 2024/0293481 A1
(43) Pub. Date: Sep. 5, 2024

(54) METHODS AND COMPOSITIONS FOR PREDICTING CANCER SURVIVAL AND CAR T CELL TOXICITY

(71) Applicants: MEMORIAL SLOAN-KETTERING CANCER CENTER, New york, NY (US); SLOAN-KETTERING INSTITUTE FOR CANCER RESEARCH, New York, NY (US); MEMORIAL HOSPITAL FOR CANCER AND ALLIED DISEASES, New York, NY (US); THE TRUSTEES OF THE UNIVERSITY OF PENNSYLVANIA, Philadelphia, PA (US)

(72) Inventors: Melody Smith, New York, NY (US); Marcel Van Den Brink, New York, NY (US); Anqi Dai, New York, NY (US); Sean Devlin, New York, NY (US); Marco Ruella, Philadelphia, PA (US); Andrea Facciabene, Philadelphia, PA (US)

(73) Assignees: MEMORIAL SLOAN-KETTERING CANCER CENTER, New york, NY (US); SLOAN-KETTERING INSTITUTE FOR CANCER RESEARCH, New York, NY (US); MEMORIAL HOSPITAL FOR CANCER AND ALLIED DISEASES, New York, NY (US); THE TRUSTEES OF THE UNIVERSITY OF PENNSYLVANIA, Philadelphia, PA (US)

(21) Appl. No.: 18/656,397

(22) Filed: May 6, 2024

Related U.S. Application Data
(63) Continuation of application No. PCT/US2022/049144, filed on Nov. 7, 2022.
(60) Provisional application No. 63/303,461, filed on Jan. 26, 2022.
(60) Provisional application No. 63/276,450, filed on Nov. 5, 2021.

(51) Int. Cl. A61K 35/742; A61K 35/17; A61K 35/745; A61K 35/747; A61K 39/00; A61K 45/06; C12Q 1/06; C12Q 1/689

(52) U.S. Cl. CPC A61K 35/742; A61K 35/17; A61K 35/745; A61K 35/747; A61K 39/4611; A61K 39/4631; A61K 39/464412; A61K 39/464417; A61K 45/06; C12Q 1/06; C12Q 1/689; C12Q 2600/106

(57) ABSTRACT

The present disclosure relates to compositions and methods for predicting cancer survival or toxicity in a subject receiving a chimeric antigen receptor (CAR) T cell therapy. The present disclosure further discloses compositions, e.g., pharmaceutical compositions, and methods for treating said subject.

### CROSS-REFERENCE TO RELATED APPLICATIONS

This application is a continuation of International Patent Application No. PCT/US22/49144, filed Nov. 7, 2022, which claims priority to U.S. Provisional Application No. 63/276,450, filed Nov. 5, 2021, and U.S. Provisional Application No. 63/303,461, filed Jan. 26, 2022, the contents of each of which are incorporated herein by reference in its entirety.

### STATEMENT OF GOVERNMENT INTEREST

This invention was made with government support under grant numbers K08CA194256, 1K99CA212302, R00CA212302, 1R01CA219871-01A1, 1P01CA214278, R01CA226983, P01 CA23766, P30 CA008748, K08HL143189, R01-CA228358, R01-CA228308, R01-HL147584, P01-CA023766, R01-HL125571, R01-HL123340 and P01-AG052359 awarded by the National Institutes of Health. The Government has certain rights in the invention.

### TECHNICAL FIELD

The present disclosure relates to compositions and methods for predicting cancer survival or toxicity in a subject receiving a chimeric antigen receptor (CAR) T cell therapy. The present disclosure further discloses compositions, e.g., pharmaceutical compositions, and methods for treating said subject.

### BACKGROUND

CD19-targeted chimeric antigen receptor (CAR) T cells have transformed the treatment of patients with relapsed or refractory CD19-positive hematologic malignancies. Four CAR T cell therapies have been approved by the Food and Drug Administration for the treatment of CD19-positive hematologic malignancies. Despite the promising results, most patients experience a lack of response or disease relapse, which can occur due to a lack of CAR T cell persistence, T cell exhaustion or loss of the target antigen due to deletions spanning the CD19 locus, CAR masking or alternative splicing. Additionally, patients can develop unique CAR-mediated toxicities that manifest either as the systemic inflammatory cytokine release syndrome (CRS) or as neurologic events, termed immune effector cell-associated neurotoxicity syndrome (ICANS).

CRS is the most frequent toxicity following CAR T cell therapy, and it results from the activity and expansion of CAR T cells upon recognition of its target antigen followed by the release of pro-inflammatory cytokines that trigger monocyte and macrophage activation with further cytokine release. The etiology of ICANS is poorly understood but has been linked to dysfunction in the blood-brain barrier, but less is known about the precise etiology. ICANS most often occurs following CRS, and its incidence ranges up to 87%. ICANS can be extremely challenging to treat and is a cause of severe morbidity and death in CAR T cell-treated patients. Overall, different CAR costimulatory domains, such as CD28 or 4-1BB, have been associated with different frequency and intensity of toxicity, with a higher incidence of severe CRS and neurotoxicity in CARs with CD28⁸. Several other factors have been associated with CAR T cell-specific toxicities. Tumor burden, intensity of lymphodepletion intensity, serum cytokine concentrations, CAR T cell dose and degree of CAR T cell expansion have been correlated with CRS, while serum cytokine concentrations, younger patient age, B-cell ALL diagnosis, high bone marrow disease burden, higher CAR T cell dose and pre-existing neurologic comorbidity have been linked to neurotoxicity^{5,16,19-21}. These risk factors, however, fail to predict with precision which patients will develop toxicities and to what degree, which raises the possibility that other variables contribute to the function of CAR T cells in vivo both with respect to their anti-tumor function and their propensity to induce toxicities.

Accordingly, there is a need in the art for methods for predicting cancer survival or CAR T cell-associated toxicity in a subject receiving a CAR T cell therapy.

### SUMMARY

The present disclosure provides for methods of determining whether a subject having a cancer has an increased likelihood or decreased likelihood to exhibit cancer survival following a CAR T cell therapy. In certain embodiments, the present disclosure provides for methods of determining whether a subject having a cancer has an increased likelihood or decreased likelihood to exhibit CAR T cell associated toxicity. The present disclosure further provides therapeutic bacteria and pharmaceutical compositions thereof for treating subjects identified as having a decreased likelihood of cancer survival and/or an increased likelihood to exhibit CAR T cell associated toxicity.

In certain embodiments, the present disclosure provides pharmaceutical compositions comprising an effective amount of a therapeutic bacteria. In certain embodiments, a pharmaceutical composition of the present disclosure can include an effective amount of a therapeutic bacterium or a spore thereof selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Streptococcus oralis, Lutispora thermophila, Agathobaculum butyriciproducens, Lactobacillus salivarius, Schaalia odontolytica, Longicatena caecimuris, Bifidobacterium breve, Ruminococcus bromii, Blautia luti, Faecalibacterium prausnitzii and a combination thereof. Alternatively or additionally, a pharmaceutical composition of the present disclosure can include an effective amount of a bacterium or a spore thereof comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Streptococcus oralis, Lutispora thermophila, Agathobaculum butyriciproducens, Lactobacillus salivarius, Schaalia odontolytica, Longicatena caecimuris, Bifidobacterium breve, Ruminococcus bromii, Blautia luti, Faecalibacterium prausnitzii or a combination thereof.

In certain embodiments, the pharmaceutical composition can include a therapeutic bacterium or a spore thereof selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti, Faecalibacterium prausnitzii and a combination thereof. In certain embodiments, the pharmaceutical composition can include a bacterium or a spore thereof comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti, Faecalibacterium prausnitzii or a combination thereof. In certain embodiments, the effective amount of the bacterium or spore thereof increases the likelihood of cancer survival in the subject administered the pharmaceutical composition.

In certain embodiments, the pharmaceutical composition can include a therapeutic bacterium or a spore thereof selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans or a combination thereof and/or a bacterium or a spore thereof comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans or a combination thereof.

In certain embodiments, the pharmaceutical composition can include a therapeutic bacterium or a spore thereof selected from the group consisting of Ruminococcus bromii, Longicatena caecimuris, Bifidobacterium breve, Blautia luti, Faecalibacterium prausnitzii or a combination thereof and/or a bacterium or a spore thereof comprising a 16S rRNA gene sequence having at least 94.5%, 95%, 97%, 98%, 98.7% or 99% sequence identity to the 16S rRNA gene sequence of Ruminococcus bromii, Longicatena caecimuris, Bifidobacterium breve, Blautia luti, Faecalibacterium prausnitzii or a combination thereof.

In certain embodiments, the pharmaceutical composition can include a therapeutic bacterium or a spore thereof selected from the group consisting of Streptococcus oralis, Lutispora thermophila, Agathobaculum butyriciproducens, Lactobacillus salivarius, Blautia luti, Faecalibacterium prausnitzii and a combination thereof. In certain embodiments, the pharmaceutical composition can include a bacterium or a spore thereof comprising a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Blautia luti, Faecalibacterium prausnitzii or a combination thereof. In certain embodiments, the effective amount of the bacterium or spore thereof decreases the likelihood of CAR T cell associated toxicity in the subject administered the pharmaceutical composition.

In certain embodiments, the pharmaceutical composition can include a therapeutic bacterium or a spore thereof selected from the group consisting of Streptococcus oralis, Lutispora thermophila, Agathobaculum butyriciproducens, Lactobacillus salivarius, Schaalia odontolytica or a combination thereof and/or a bacterium or a spore thereof comprising a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica or a combination thereof.

In certain embodiments, the pharmaceutical composition can include a therapeutic bacterium or a spore thereof selected from the group consisting of Blautia luti, Faecalibacterium prausnitzii or a combination thereof and/or a bacterium or a spore thereof comprising a 16S rRNA gene sequence having at least 94.5%, 95%, 97%, 98%, 98.7% or 99% sequence identity to the 16S rRNA gene sequence of Blautia luti, Faecalibacterium prausnitzii or a combination thereof.

In certain embodiments, the pharmaceutical composition can further include a biocompatible pharmaceutical carrier. In certain embodiments, the pharmaceutical composition can further include a probiotic bacteria, a probiotic yeast, a prebiotic, a postbiotic, an antibiotic or a combination thereof. In certain embodiments, the pharmaceutical composition is formulated for oral, nasogastric, rectal, percutaneous or gastric tube administration. In certain embodiments, the pharmaceutical composition is in a form of a liquid, a suspension, a dried powder, a tablet, a capsule, a food product or a combination thereof. In certain embodiments, the therapeutic bacterium is a recombinant bacterium or a progeny thereof.

The present disclosure further provides methods for identifying a subject as having a decreased likelihood of cancer survival following a CAR T cell therapy. In certain embodiments, the method can include (a) determining a level of a bacterium or a spore thereof in a sample of the subject; (b) comparing the level of the bacterium or spores thereof to a reference level of the bacterium or a spore thereof, wherein the reference level is the level of the bacterium or a spore thereof in a patient that does not exhibit cancer survival; and (c) identifying the subject as having a decreased likelihood of cancer survival if the level of the bacterium or spore thereof is higher than the reference level. In certain embodiments, the bacterium or spore thereof is selected from the group consisting of Hungatella effluvii, Coprobacillus cateniformis, Atopobium parvulum, Faecalicoccus acidiformans, Lactococcus lactis, Anaerobacterium chartisolvens, Atopobiaceae Clostridium innocuum, Bacteroides thetaiotaomicron, a species of the Veillonellaceae family and a combination thereof; or the bacterium or spore thereof comprises a 16S rRNA gene sequence having at least 94.5%, 95%, 97%, 98%, 98.7% or 99% sequence identity to the 16S rRNA gene sequence of Hungatella effluvii, Coprobacillus cateniformis, Atopobium parvulum, Faecalicoccus acidiformans, Lactococcus lactis, Anaerobacterium chartisolvens, Atopobiaceae Clostridium innocuum, Bacteroides thetaiotaomicron, a species of the Veillonellaceae family or a combination thereof. In certain embodiments, the bacterium or spore thereof is a species of the Veillonellaceae family or is a bacterium or spore thereof comprising a 16S rRNA gene sequence having at least 94.5%, 95%, 97%, 98%, 98.7% or 99% sequence identity to the 16S rRNA gene sequence of a species of the Veillonellaceae family.

The present disclosure further provides methods for identifying a subject having an increased likelihood of exhibiting a CAR T cell associated toxicity. In certain embodiments, the method can include (a) determining a level of a bacterium or a spore thereof in a sample of the subject; (b) comparing the level of the bacterium or spores thereof to a reference level of the bacterium or a spore thereof, wherein the reference level is the level of the bacterium or a spore thereof in a patient that exhibits a CAR T cell associated toxicity; and (c) identifying the subject as having an increased likelihood of exhibiting a CAR T cell associated toxicity if the level of the bacterium or spore thereof is higher than the reference level. In certain embodiments, the bacterium or spore thereof is selected from the group consisting of Streptococcus salivarius, Oscillibacter ruminantium, Eubacterium ramulus, Streptococcus gordonii, Caprocicproducens galactitolivorans, Clostridium hylemonae and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus salivarius, Oscillibacter ruminantium, Eubacterium ramulus, Streptococcus gordonii, Caprocicproducens galactitolivorans, Clostridium hylemonae and a combination thereof.

The present disclosure further provides methods for identifying a subject as having an increased likelihood of cancer survival following a CAR T cell therapy. In certain embodiments, a method for identifying a subject having a cancer as having an increased likelihood of cancer survival following a CAR T cell therapy includes (a) determining a level of a bacterium or a spore thereof in a sample of the subject; (b) comparing the level of the bacterium or spores thereof to a reference level of the bacterium or a spore thereof, wherein the reference level is the level of the bacterium or a spore thereof in a patient exhibited cancer survival; and (c) identifying the subject as having increased likelihood of cancer survival if the level of the diagnostic bacterium or spore thereof is higher than the reference level. In certain embodiments, the bacterium or spore thereof is selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Longicatena caecimuris, Bifidobacterium breve, Escherichia coli, Clostridium methoxybenzovorans, Ruminococcus bromii, Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Longicatena caecimuris, Bifidobacterium breve, Escherichia coli, Clostridium methoxybenzovorans, Ruminococcus bromii, Blautia luti, Faecalibacterium prausnitzii or a combination thereof.

The present disclosure further provides methods for identifying a subject having a decreased likelihood of exhibiting a CAR T cell associated toxicity. In certain embodiments, the method can include (a) determining a level of a bacterium or a spore thereof in a sample of the subject; (b) comparing the level of the bacterium or spores thereof to a reference level of the bacterium or a spore thereof, wherein the reference level is the level of the bacterium or a spore thereof in a patient that did not exhibit a CAR T cell associated toxicity; and (c) identifying the subject as having a decreased likelihood of exhibiting a CAR T cell associated toxicity if the level of the bacterium or spore thereof is higher than the reference level. In certain embodiments, the bacterium or spore thereof is selected from the group consisting of Streptococcus oralis, Lutispora thermophila, Agathobaculum butyriciproducens, Lactobacillus salivarius, Schaalia odontolytica, Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica, Blautia luti, Faecalibacterium prausnitzii and a combination thereof. In certain embodiments, the bacterium or spore thereof is selected from the group consisting of Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Blautia luti, Faecalibacterium prausnitzii and a combination thereof. In certain embodiments, the bacterium or spore thereof is selected from the group consisting of Streptococcus oralis, Lutispora thermophila, Agathobaculum butyriciproducens, Lactobacillus salivarius, Schaalia odontolytica and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica and a combination thereof.

In certain embodiments, a method for identifying a subject having a cancer as having an increased likelihood of exhibiting cancer survival can include (a) determining a level of a bacterial gene in a sample of the subject; (b) comparing the level of the bacterial gene to a reference bacterial gene level, wherein the reference level is the level of the bacterial gene in a patient that exhibited cancer survival; and (c) identifying the subject as having an increased likelihood of exhibiting cancer survival if the level of the bacterial gene is higher than the reference bacterial gene level. In certain embodiments, the bacterial gene is a gene involved in aromatic amino acid biosynthesis and/or peptidoglycan biosynthesis.

In certain embodiments, a method for identifying a subject having a cancer as having a decreased likelihood to exhibit cancer survival and/or an increased likelihood to exhibit a CAR T cell associated toxicity can include (a) determining if the subject has been administered an antibiotic; and (b) identifying the subject as having an increased likelihood of a CAR T cell associated toxicity and/or a decreased likelihood of cancer survival if the subject has been administered piperacillin-tazobactam, imipenem-cilastatin, meropenem or a combination thereof. In certain embodiments, the antibiotic is administered less than about 4 weeks prior to the initiation of a CAR T cell therapy.

In certain embodiments, any one of the methods disclosed herein can include treating the subject identified as having a decreased likelihood of cancer survival or as having an increased likelihood of exhibiting a CAR T cell associated toxicity with a pharmaceutical composition disclosed herein. In certain embodiments, any one of the methods disclosed herein can include treating the subject with a CAR T cell therapy.

The present disclosure further provides methods for treating a subject having a cancer with a CAR T cell therapy. In certain embodiments, the method can include (a) determining a level of a bacterium or a spore thereof in a sample of the subject; (b) comparing the level of the bacterium or spores thereof to a reference level of the bacterium or a spore thereof, wherein the reference level is the level of the bacterium or a spore thereof in a subject that exhibited cancer survival; (c) identifying the subject as having an increased likelihood of cancer survival if the level of the bacterium or spore thereof is higher than the reference level; and (d) treating the subject identified as having an increased likelihood of cancer survival with a CAR T cell therapy. In certain embodiments, the bacterium or spore thereof is selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Blautia luti, Longicatena caecimuris, Bifidobacterium breve, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Blautia luti, Longicatena caecimuris, Bifidobacterium breve and/or Faecalibacterium prausnitzii.

In certain embodiments, a method for treating a subject having a cancer with a CAR T cell therapy can include (a) determining a level of a bacterium or a spore thereof in a sample of the subject; (b) comparing the level of the bacterium or spores thereof to a reference level of the bacterium or a spore thereof, wherein the reference level is the level of the bacterium or a spore thereof in a subject that did not exhibit cancer survival; (c) identifying the subject as having a decreased likelihood of cancer survival if the level of the bacterium or spore thereof is higher than the reference level; and (d) treating the subject identified as having a decreased likelihood of cancer survival with a pharmaceutical composition comprising one or more therapeutic bacteria and a CAR T cell therapy. In certain embodiments, the bacterium or spore thereof is selected from the group consisting of Hungatella effluvii, Coprobacillus cateniformis, Atopobium parvulum, Faecalicoccus acidiformans, Lactococcus lactis, Anaerobacterium chartisolvens, Clostridium innocuum, Bacteroides thetaiotaomicron, a species of the Veillonellaceae family and a combination thereof; or the bacterium or spore thereof comprises a 16S rRNA gene sequence having at least 94.5%, 95%, 97%, 98%, 98.7% or 99% sequence identity to the 16S rRNA gene sequence of Hungatella effluvii, Coprobacillus cateniformis, Atopobium parvulum, Faecalicoccus acidiformans, Lactococcus lactis, Anaerobacterium chartisolvens, Clostridium innocuum, Bacteroides thetaiotaomicron, a species of the Veillonellaceae family and a combination thereof. In certain embodiments, the therapeutic bacteria comprises one or more of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti, Faecalibacterium prausnitzii or a bacteria comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti and/or Faecalibacterium prausnitzii.

In certain embodiments, a method for treating a subject having a cancer with a CAR T cell therapy can include (a) determining a level of a bacterium or a spore thereof in a sample of the subject; (b) comparing the level of the bacterium or spores thereof to a reference level of the bacterium or a spore thereof, wherein the reference level is the level of the bacterium or a spore thereof in a subject that did not exhibit a CAR T cell associated toxicity; (c) identifying the subject as having a decreased likelihood of exhibiting a CAR T cell associated toxicity if the level of the bacterium or spore thereof is higher than the reference level; and (d) treating the subject identified as having a decreased likelihood of exhibiting a CAR T cell associated toxicity with a CAR T cell therapy. In certain embodiments, the bacterium or spore thereof is selected from the group consisting of Streptococcus oralis, Lutispora thermophila, Schaalia odontolytica, Agathobaculum butyriciproducens, Lactobacillus salivarius, Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus oralis, Lutispora thermophila, Schaalia odontolytica, Agathobaculum butyriciproducens, Lactobacillus salivarius, Blautia luti and/or Faecalibacterium prausnitzii.

In certain embodiments, a method for treating a subject having a cancer with a CAR T cell therapy can include (a) determining a level of a bacterium or a spore thereof in a sample of the subject; (b) comparing the level of the bacterium or spores thereof to a reference level of the bacterium or a spore thereof, wherein the reference level is the level of the bacterium or a spore thereof in a subject that exhibited CAR T cell associated toxicity; (c) identifying the subject as having an increased likelihood of exhibiting a CAR T cell associated toxicity if the level of the bacterium or spore thereof is higher than the reference level; and (d) treating the subject identified as having an increased likelihood of exhibiting a CAR T cell associated toxicity with a pharmaceutical composition comprising one or more therapeutic bacteria and a CAR T cell therapy. In certain embodiments, the bacterium or spore thereof is selected from the group consisting of Streptococcus salivarius, Oscillibacter ruminantium, Eubacterium ramulus, Streptococcus gordonii, Caprocicproducens galactitolivorans, Clostridium hylemonae and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus salivarius, Oscillibacter ruminantium, Eubacterium ramulus, Streptococcus gordonii, Caprocicproducens galactitolivorans, Clostridium hylemonae or a combination thereof. In certain embodiments, the therapeutic bacteria comprises one or more of Streptococcus oralis, Lutispora thermophila, Agathobaculum butyriciproducens and/or Lactobacillus salivarius, Schaalia odontolytica, Blautia luti, Faecalibacterium prausnitzii or a bacterium comprising a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica, Blautia luti, Faecalibacterium prausnitzii or a combination thereof.

In certain embodiments, any one of the methods disclosed herein can include administering to the subject a CAR T cell therapy. In certain embodiments, any one of the methods disclosed herein can include administering to the subject a chemotherapy, immunotherapy, stem cell therapy, cellular therapy, a probiotic bacteria, a probiotic yeast, a prebiotic, a postbiotic, an antibiotic or a combination thereof. In certain embodiments, cancer survival is the survival of the subject at least about 100 days following a CAR T cell therapy. In certain embodiments, a CAR T cell associated toxicity is cytokine release syndrome (CRS) and/or immune effector cell-associated neurotoxicity syndrome (ICANS). In certain embodiments, the sample is a fecal sample or an intestinal content sample of the subject. In certain embodiments, the cancer is selected from the group consisting of acute lymphoblastic leukemia (ALL), chronic lymphoblastic leukemia (CLL) or non-Hodgkin lymphoma (NHL)). In certain embodiments, the CAR T cell therapy includes a CAR T cell that targets mucin 16 (MUC16), B-cell maturation antigen (BCMA) and/or CD19.

The present disclosure further provides kits comprising any one of the disclosed therapeutic bacteria or pharmaceutical compositions thereof. The present disclosure further provides kits for performing any one of the disclosed methods. For example, but not by way of limitation, the kit can include means for identifying a bacterium or a spore thereof selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Hungatella effluvii, Coprobacillus cateniformis, Atopobium parvulum, Faecalicoccus acidiformans, Lactococcus lactis, Anaerobacterium chartisolvens, Atopobiaceae, Clostridium innocuum, Bacteroides thetaiotamicron, Streptococcus salivarius, Oscillibacter ruminantium, Eubacterium ramulus, Streptococcus gordonii, Caprocicproducens galactitolivorans, Clostridium hylemonae, Streptococcus oralis, Lutispora thermophila, Agathobaculum butyriciproducens, Schaalia odontolytica, Lactobacillus salivarius, Ruminococcus bromii, Bifidobacterium breve, Longicatena caecimuris, Escherichia coli, Blautia luti, Faecalibacterium prausnitzii and a combination thereof.

In certain embodiments, a kit of the present disclosure can further include instructions for treating a subject having a cancer. In certain embodiments, the kit can include instructions for identifying the subject as having an increased likelihood or decreased likelihood of exhibiting cancer survival. In certain embodiments, the instructions comprise (a) determining the level of the bacterium or spore thereof in a sample of the subject; (b) comparing the level of the bacterium or spores thereof to a reference level of the bacterium or a spore thereof; and (c) identifying the subject as having an increased likelihood or decreased likelihood of exhibiting cancer survival based on the comparison. In certain embodiments, the kit can include instructions for identifying the subject as having an increased likelihood or decreased likelihood of a CAR T cell associated toxicity. In certain embodiments, the instructions comprise (a) determining the level of the bacterium or spore thereof in a sample of the subject; (b) comparing the level of the bacterium or spores thereof to a reference level of the bacterium or a spore thereof; and (c) identifying the subject having an increased likelihood or decreased likelihood of a CAR T cell associated toxicity based on the comparison.

The present disclosure further provides uses for the pharmaceutical compositions of the present disclosure. In certain embodiments, the present disclosure provides a use of a pharmaceutical composition disclosed herein for treating a subject having a cancer. In certain embodiments, the present disclosure provides a use of a pharmaceutical composition disclosed herein for increasing the likelihood of cancer survival in a subject. In certain embodiments, the present disclosure provides a use of a pharmaceutical composition disclosed herein for decreasing the likelihood of a CAR T cell associated toxicity in a subject. In certain embodiments, the cancer and/or subject is being treated or will be treated with a CAR T cell therapy.

### BRIEF DESCRIPTION OF THE FIGURES

FIGS. 1A-1H. Impact of antibiotic exposure in patients with hematologic malignancies treated with anti-CD19 CAR T cell therapy. (A) The antibiotic cohort consists of CD19 CAR T cell recipients from MSK (n=127) and Penn (n=101) who were assessed in a retrospective observational study of antibiotic exposure (left panel) (N=228). Frequency of antibiotic exposure in the four weeks prior to CAR T cell infusion in patients with NHL and ALL (right panel) (N=228). Percent frequency of exposure to piperacillin/tazobactam, imipenem/cilastatin and meropenem are highlighted in yellow indicating the antibiotics that constitute P-I-M (also noted in yellow). Percent frequency of exposure to cefepime is noted in green. The blue graph denotes the cumulative exposure to any antibiotic. “Others” encompasses all the antibiotics that were administered in only one patient each (clindamycin, cephalexin, tobramycin, cefpodoxime, atovaquone, ampicillin/sulbactam, cefuroxime, cefazolin). (B) Kaplan-Meier overall survival curves by log-rank test in ALL and NHL populations according to exposure to any antibiotic within the 4 weeks before CD19 CAR T cell infusion (N=228). The gray line indicates patients not exposed to any antibiotic treatment, while the yellow line indicates patients exposed to any antibiotic treatment. (C, D, E) Kaplan-Meier curves of progression-free survival (PFS) and overall survival (OS) by log-rank test according to the exposure to P-I-M antibiotics the within 4 weeks before CD19 CAR T cell infusion in patients with ALL and NHL (only OS, N=228), NHL (n=137) and ALL (n=91), respectively. The gray line indicates patients not exposed to P-I-M antibiotics, while the yellow line indicates patients exposed to P-I-M antibiotics. For Kaplan-Meier analysis, the points represent data censored at the last time the patient was known to be alive and without progression. The shading indicates the 95% confidence interval, and the tick marks indicate censored events. (F, G, H) Histograms of the frequencies of any grade CRS and ICANS by Wilcoxon rank-sum test according to exposure to P-I-M antibiotics within 4 weeks before CD19 CAR T cell infusion in patients with ALL and NHL (N=228), NHL (n=137) and ALL (n=91), respectively. Blue indicates the absence of CRS or ICANS of any grade, while red indicates the presence of CRS or ICANS of any grade. Abbreviations: Trimeth./Sulfameth.: trimethoprim/sulfamethoxazole; IV: intravenous; NHL: non-Hodgkin lymphoma; Not exposed: patients exposed to non-P-I-M plus patients who did not receive any antibiotics within the 4 weeks before CD19 CAR T cell infusion; ALL: acute lymphoblastic leukemia; p: p-value; P-I-M: exposure to either piperacillin/tazobactam, imipenem/cilastatin or meropenem within the 4 weeks before CD19 CAR T cell infusion; CRS: cytokine releasing syndrome; ICANS: immune effector cell-associated neurotoxicity

FIGS. 2A-2C. Impact of antibiotic exposure in patients with hematologic malignancies treated with anti-CD19 CAR T cell therapy according to institution. (A) Frequency of antibiotic exposure in the four weeks prior to CD19 CAR T cell infusion in patients with NHL and ALL treated at MSK (upper panel, n=127) and Penn (bottom panel, n=101). Purple denotes patients with ALL, while orange denotes patients with NHL. (B and C) Kaplan-Meier curves of overall survival (OS) by log-rank test according to the exposure to P-I-M antibiotics within 4 weeks before CD19 CAR T cell infusion in patients with ALL and NHL treated at MSK (B, n=127) and Penn (C, n=101). The gray line indicates patients not exposed to P-I-M antibiotics, while the yellow line indicates patients exposed to P-I-M antibiotics. For Kaplan-Meier analysis, the points represent data censored at the last time the patient was known to be alive and without progression. The shading indicates the 95% confidence interval, and the tick marks indicate censored events. Abbreviations: Trimeth./Sulfameth.: trimethoprim/sulfamethoxazole; IV: intravenous; NHL: non-Hodgkin lymphoma; ALL: acute lymphoblastic leukemia; MSK: Memorial Sloan Kettering Cancer Center; Penn: University of Pennsylvania; P-I-M: exposure to either piperacillin/tazobactam, imipenem/cilastatin or meropenem within the 4 weeks before CD19 CAR T cell infusion; Not exposed: patients exposed to non-P-I-M plus patients who did not receive any antibiotics; IV: intravenous; p: p-value.

FIGS. 3A-3B. Survival analysis comparison of different antibiotics exposure on non-Hodgkin lymphoma patients treated with CD19 CAR T cells. (A and B) Kaplan-Meier curves of (A) progression-free survival (PFS) and (B) overall survival (OS) by log-rank test. Data shows the combined NHL population (n=137) treated with different antibiotics in the 4 weeks before CD19 CAR T cell infusion. The gray line indicates patients not exposed to P-I-M antibiotics or cefepime (n=107), the yellow line indicates patients exposed to P-I-M antibiotics (n=21), and the green line indicates patients not exposed to P-I-M antibiotics and exposed to cefepime (n=9). The points represent data censored at the last time the patient was known to be alive and without progression. The shading indicates the 95% confidence interval, and the tick marks indicate censored events. P values are shown (log-rank analysis). Abbreviations: NHL: non-Hodgkin lymphoma; P-I-M: exposure to either piperacillin/tazobactam, imipenem/cilastatin or meropenem within the 4 weeks before CD19 CAR T cell infusion; No P-I-M antibiotic exposure: patients exposed to non-P-I-M plus patients who did not receive any antibiotics within 4 weeks before CD19 CAR T cell infusion; p: p-value.

FIGS. 4A-4D. Impact of P-I-M antibiotics exposure in patients with non-Hodgkin lymphoma treated with anti-CD19 CAR T cell according to CAR-costimulatory domain. (A and B) Kaplan-Meier curves of (A) progression-free survival (PFS) and (B) overall survival (OS) by log-rank test according to the exposure to P-I-M antibiotics within 4 weeks before CD19 CAR T cell infusion in a NHL population treated with CD19 CAR T cells with a CD28 costimulatory domain (A; n=72) and CD19 CAR T cells with a 4-1BB costimulatory domain (B; n=65). The gray line indicates patients not exposed to P-I-M antibiotics, while the yellow line indicates patients exposed to P-I-M antibiotics. The points represent data censored at the last time the patient was known to be alive and without progression. The shading indicates the 95% confidence interval, and the tick marks indicate censored events. P values are shown (log-rank analysis). (C and D) Histograms show the frequencies of CRS and ICANS by Wilcoxon rank-sum test according to exposure to P-I-M antibiotics within the 4 weeks before CD19 CAR T cell infusion in patients with NHL who received (C) a product with a CD28 costimulatory domain and patients with NHL who received (D) a product with a 4-1BB costimulatory domain. Blue indicates the absence of CRS or ICANS of any grade, while red indicates the presence of CRS or ICANS of any grade. Abbreviations: NHL: non-Hodgkin lymphoma; P-I-M: exposure to either piperacillin/tazobactam, imipenem/cilastatin or meropenem within the 4 weeks before CD19 CAR T cell infusion; No P-I-M antibiotic exposure: patients exposed to non-P-I-M plus patients who did not receive any antibiotics within 4 weeks before CD19 CAR T cell infusion; PFS: progression-free survival; OS: overall survival: p: p-value; CRS: cytokine releasing syndrome; ICANS: immune effector cell-associated neurotoxicity

FIGS. 5A-5Q. The association of fecal microbiota with clinical response in recipients of CD19 CAR T cells. (A to J) Data presented in these panels are based on 16S rRNA gene sequencing data. (A) Schema of fecal sample collection and sequencing analyses. (B) Characterization of baseline stool samples according to their phylogenetic composition in stacked bar plot of bacterial taxa (n=45). Red asterisks denote samples with domination, defined as a relative abundance of at least 30% for any taxonomic unit. (C) Inverse Simpson diversity index of the fecal microbiome in the baseline fecal samples (n=45) and healthy volunteers (n=30) by Wilcoxon rank-sum test. This healthy volunteer cohort has been investigated in a prior published study. The middle line is the median, the box limits represent the upper and lower quartiles, the whiskers note 1.5× the interquartile range, and the dots represent the individual data points. (D) Fecal microbiome composition of the CAR T cell patients and healthy volunteers displayed in a PCoA. Composition assessed using beta-diversity calculated with Bray-Curtis dissimilarity. Data visualized at the ASV level. Red dots indicate CAR T cells patients and green dots indicate healthy volunteers. (E) Plot of the estimated coefficient for Inverse Simpson diversity index of the baseline fecal microbiome and Day 100 CR and toxicity using Bayesian logistic regression. Error bars represent the 95% credibility interval, and the dots represent the point estimate. (F, G) Linear discriminant analysis (LDA) scores computed for differentially abundant taxa in the baseline fecal microbiome samples from 16S sequencing of Day 100 CR (green) and no Day 100 CR (purple) as well as no toxicity (blue). Length indicates effect size associated with a taxon. LDA score>4. (H, J) Plot of the estimated coefficients for log 10 relative abundance of bacterial taxa at the genus level in the baseline fecal sample for Day 100 CR and toxicity. Error bars represent the 95% credibility interval, and the dots represent the point estimate. (I, K) Patient samples with highest 10% (red) or lowest 10% (blue) relative abundance of genera. Predicted probability of (H) Day 100 CR at different abundances of Ruminococcus and (J) predicted probability of toxicity at different abundances Bacteroides. (L, M) Data presented in these panels is based on metagenomic shotgun sequencing. LDA score of computed for differentially abundant pathways in the baseline fecal microbiome samples from metagenomic shotgun sequencing of Day 100 CR (green) and no Day 100 CR (purple) as well as toxicity (red) and no toxicity (blue). Length indicates effect size associated with a taxon. LDA score>2. (N, O) Linear discriminant analysis (LDA) scores computed for differentially abundant taxa in the baseline fecal microbiome samples from 16S sequencing of Day 100 CR (green) and no Day 100 CR (purple) as well as toxicity (red) and no toxicity (blue). Length indicates effect size associated with a taxon. LDA score>2. (P) LDA of scores computed for differentially abundant taxa in the baseline fecal microbiome samples from metagenomic shotgun sequencing of Day 100 CR (green) and no Day 100 CR (purple). (Q) LDA score of computed for differentially abundant pathways in the baseline fecal microbiome samples from metagenomics. The healthy volunteer cohort was used in a prior publication. Abbreviations: LDA: Linear discriminant analysis; ASV: amplicon sequence variant; NHL: non-Hodgkin lymphoma; P-I-M: exposure to either piperacillin/tazobactam, imipenem/cilastatin or meropenem within the 4 weeks before CD19 CAR T cell infusion; PFS: progression-free survival; OS: overall survival: p: p-value.

FIG. 6. Flow diagram of the fecal microbiome sample collection. Fifty-one unique patients were collected upon informed consent. Of the fifty-one patients, one patient did not have sufficient fecal material for sequencing and two patients failed during the amplification or quality control step. Following these exclusions, there were forty-eight patients in the fecal microbiome cohort. Of these patients, the 16S ribosomal RNA gene was successfully amplified and sequenced with >200 reads per sample from forty-five patients. Forty-five patients passed quality control measures for metagenomic shotgun sequencing. There were three non-overlapping patients in the 16S and shotgun sequencing cohorts. Hence, there were 48 unique patients in the fecal microbiome cohort.

FIGS. 7A-7E. The association of intestinal microbiota and clinical response in recipients of CD19 CAR T cells, including subset analysis institution. (A to E) All data reported in this figure is based on 16S rRNA gene sequencing data. (A) Inverse Simpson diversity index of the fecal microbiome in the baseline fecal samples by institution, MSK (n=26) and Penn (n=19), compared to healthy volunteers (n=30) by Wilcoxon rank-sum test. The middle line is the median, the box limits represent the upper and lower quartiles, the whiskers note 1.5× the interquartile range, and the dots represent the individual data points. (B to C) Beta-diversity was calculated using the Bray-Curtis dissimilarity between a reference point defined by the average of healthy volunteers and each of 30 samples from healthy volunteers. Healthy volunteers were compared to the 45 baseline patient samples (B) and by institution (MSK n=26; Penn n=19) (C) by Wilcoxon rank-sum test. This healthy volunteer cohort has been investigated in a prior published study. The middle line is the median, the box limits represent the upper and lower quartiles, the whiskers note 1.5× the interquartile range, and the dots represent the individual data points. (D to E) Patient samples with higher (one standard deviation above the mean) (red) or lower (one standard deviation below the mean) (blue) Inverse Simpson diversity index. The coefficients for the predicted probability of (C) Day 100 CR and (D) toxicity by Inverse Simpson diversity index. The coefficients correspond to the Bayesian models for Day 100 CR and toxicity, respectively, in FIG. 5E.

FIGS. 8A-8C. Impact of any antibiotic exposure in patients with non-Hodgkin lymphoma treated with anti-CD19 CAR T cell therapy. (A and B) Kaplan-Meier (A) progression-free (PFS) and (B) overall survival (OS) curves by log-rank test in NHL populations according to exposure to any antibiotic within 4 weeks before CD19 CAR T cell infusion (n=137). The gray line indicates patients not exposed to any antibiotic treatment, while the yellow line indicates patients exposed to any antibiotic treatment. (C) Histograms of the frequencies of any grade CRS and ICANS by Wilcoxon rank-sum test according to the exposure to any antibiotic within the 4 weeks before CD19 CAR T cell infusion in patients with NHL (n=137). Blue indicates the absence of CRS or ICANS of any grade, while red indicates the presence of CRS or ICANS of any grade. Abbreviations: NHL: non-Hodgkin lymphoma; p: p-value; CRS: cytokine releasing syndrome; ICANS: immune effector cell-associated neurotoxicity.

FIGS. 9A-9B. Survival analysis comparison of piperacillin/tazobactam compared to cefepime exposure in non-Hodgkin lymphoma patients treated with CD19 CAR T cells. (A and B) Kaplan-Meier curves of (A) progression-free survival (PFS) and (B) overall survival (OS) by log-rank test. Data shows patients from the combined NHL population treated with piperacillin/tazobactam or cefepime in the 4 weeks before CD19 CAR T cell infusion. The blue line indicates patients exposed to piperacillin/tazobactam (n=18) and the green line shows patients exposed to cefepime (n=12). The points represent data censored at the last time the patient was known to be alive and without progression. The shading indicates the 95% confidence interval, and the tick marks indicate censored events. P values are shown (log-rank analysis). The p-values are not stratified by Center. Abbreviations: NHL: non-Hodgkin lymphoma; p: p-value.

FIGS. 10A-10B. Survival analysis comparison of P-I-M versus non-P-I-M exposure on non-Hodgkin lymphoma patients treated with CD19 CAR T cells. (A and B) Kaplan-Meier curves of (A) progression-free survival (PFS) and (B) overall survival (OS) by log-rank test. Data shows patients from the combined NHL population treated with P-I-M or non-P-I-M antibiotics in the 4 weeks before CD19 CAR T cell infusion. Patients who did not receive any antibiotic in the 30 days prior to CAR T cell infusion are excluded from this analysis. The gray line indicates patients exposed to P-I-M (n=21) and the yellow line shows patients exposed to non-P-I-M (n=60). The points represent data censored at the last time the patient was known to be alive and without progression. The shading indicates the 95% confidence interval, and the tick marks indicate censored events. P values are shown (log-rank analysis). The p-values are not stratified by Center. Abbreviations: NHL: non-Hodgkin lymphoma; p: p-value

FIG. 11. Timing of fecal sample collection relative to the start of conditioning chemotherapy and CD19 CAR T cell infusion. Forty-eight patients were evaluated in the fecal microbiome cohort. Of the forty-eight patients, the fecal samples of fourteen were collected before the start of conditioning chemotherapy, whereas thirty-four fecal samples were collected after the start of conditioning chemotherapy. All the baseline fecal microbiome samples were collected prior to CD19 CAR T cell infusion. The red square denotes the start of conditioning chemotherapy. The black circle denotes the collection of the baseline fecal sample prior to CAR T cell infusion. Day 0 denotes the day of CD19 CAR T cell infusion.

FIG. 12. Principal Coordinates Analysis (PCoA) visualization of beta-diversity of fecal samples of CAR T cell patients and healthy volunteers. Fecal microbiome composition of the CAR T cell patients and healthy volunteers was displayed in a PCoA. Composition was assessed using beta-diversity calculated with Bray-Curtis dissimilarity. Data visualized at the genus level. Red dots indicate CAR T cells patients and green dots indicate healthy volunteers. All data reported in this figure is based on 16S rRNA gene sequencing data.

FIG. 13. Boxplots of the relative abundance of selected taxa from LEfSe of Day 100 CR. The relative abundance of Bacteroides, Bifidobacterium, Blautia, Faecalibacterium, Longicatena, and Ruminococcus are presented. Data is categorized by patients who did not achieve a Day 100 CR (No), and patients who achieved a Day 100 CR (Yes). Dots indicate relative abundance of the baseline fecal sample from a CAR T cell patient. Wilcoxon rank-sum test was used to calculate the p-values, and the p-values were adjusted for multiple hypothesis testing.

FIG. 14. Boxplots of the relative abundance of selected taxa from LEfSe of toxicity. The relative abundance of Bacteroides, Blautia, Faecalibacterium, and Ruminococcus are presented. Data is categorized by patients who did not experience toxicity (No), and patients who experienced toxicity (Yes). Dots indicate relative abundance of the baseline fecal sample from a CAR T cell patient. Wilcoxon rank-sum test was used to calculate the p-values, and the p-values were adjusted for multiple hypothesis testing.

FIG. 15. Principal Coordinates Analysis (PCoA) visualization of pools of 16S sequencing of baseline fecal samples from CAR T cell patients. Composition diversity of baseline patient samples (n=45) is displayed according to principal coordinates analysis (PCoA). Dots are colored to indicate the sequencing pools (n=13). All data reported in this figure is based on 16S rRNA gene sequencing data.

FIG. 16. Correlation between the centered log-ratio and the log 10 transformed counts for the five genera. Scatter plots show the Pearson correlation between the centered log-ratio and the log 10 transformed counts for the five genera in the Bayesian model. All data reported in this figure are based on 16S rRNA gene sequencing data.

### DETAILED DESCRIPTION

The present disclosure relates to compositions and methods for identifying subjects considered for or undergoing a chimeric antigen receptor (CAR) T cell therapy who are more or less likely to exhibit cancer survival following CAR T cell therapy or who are more or less likely to exhibit CAR T cell therapy associated toxicity by analyzing the intestinal microbiome of those subjects before or during the CAR T cell therapy. The present disclosure further provides therapeutic bacteria and methods for improving the likelihood of cancer survival following CAR T cell therapy and/or decreasing the likelihood of CAR T cell therapy associated toxicity in a subject. The present disclosure is based, in part, on the discovery that the likelihood of cancer survival after CAR T cell therapy or the likelihood of CAR T cell toxicity is associated with the presence and/or high abundance of specific bacteria. Surprisingly, this association is generalizable across treatment centers and CAR T cell therapies.

For clarity of description, and not by way of limitation, this section is divided into the following subsections:
I. Definitions;
II. Diagnostic Methods;
III. Therapeutic Bacteria;
IV. Pharmaceutical Compositions;
V. Methods of Treatment;
VI. Kits; and
VII. Exemplary Embodiments

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” or “patient” herein is a vertebrate, such as a human or non-human animal, for example, a mammal. Mammals include, but are not limited to, humans, primates, farm animals, sport animals, rodents and pets. Non-limiting examples of non-human animal subjects include birds, such a poultry, including chickens, turkeys, ducks, and geese; rodents such as mice, rats, hamsters, and guinea pigs; rabbits; dogs; cats; sheep; pigs; goats; cattle; horses; and non-human primates such as apes and monkeys.

“Microbiota” refers to the communities of microbes that live in or on an individual's body, both sustainably and transiently, including eukaryotes, archaea, bacteria and viruses (including bacterial viruses (i.e., phage)).

“Microbiome” refers to the genetic content of the communities of microbes that live in and on the human body, both sustainably and transiently, including eukaryotes, archaea, bacteria, and viruses (including bacterial viruses (i.e., phage)), wherein “genetic content” includes genomic DNA, RNA such as micro RNA and ribosomal RNA (rRNA), the epigenome, plasmids, and all other types of genetic information.

The term “isolated,” as used herein, can refer to a bacterium or other substance or organism that has been separated from at least some of the components with which it was associated when initially produced (whether in nature or in an experimental setting) and/or produced, prepared, purified and/or manufactured. In certain embodiments, isolated bacteria can be separated from at least about 10%, about 20%, about 30%, about 40%, about 50%, about 60%, about 70%, about 80%, about 90% or more of the other components with which they were initially associated. In certain embodiments, isolated bacteria are more than about 80%, about 85%, about 90%, about 91%, about 92%, about 93%, about 94%, about 95%, about 96%, about 97%, about 98%, about 99% or more than about 99% pure. In certain embodiments, a substance or organism, e.g., bacterium, is “pure” if it is substantially free of other components.

The terms “purify,” “purifying” and “purified,” as used herein, can refer to a bacterium or other material that has been separated from at least some of the components with which it was associated either when initially produced or generated (e.g., whether in nature or in an experimental setting), or during any time after its initial production. In certain embodiments, a bacterium or a bacterial population can be considered purified if it is isolated at or after production, such as from a material or environment containing the bacterium or bacterial population, and a purified bacterium or bacterial population can contain other materials up to about 10%, about 20%, about 30%, about 40%, about 50%, about 60%, about 70%, about 80%, about 90%, or above about 90% and still be considered “isolated.” In certain embodiments, purified bacteria and bacterial populations are more than about 80%, about 85%, about 90%, about 91%, about 92%, about 93%, about 94%, about 95%, about 96%, about 97%, about 98%, about 99% or more than about 99% pure. In the certain embodiments of the bacterial compositions described herein, the one or more bacteria present in the composition can be independently purified from one or more other bacteria produced and/or present in the material or environment containing the bacteria species.

“CAR T cell therapy,” as used herein, refers to the killing of cancer cells using a T cell genetically modified to express a chimeric antigen receptor (CAR) that binds to the cancer cells, resulting in activation of the patient's immune system to kill the cancer cells. CAR T cell therapy can be particularly useful in treating acute lymphoblastic leukemia (ALL), non-Hodgkin lymphoma (NHL), CD19 malignancies, myeloma or other B cell-related or hematologic malignancies, or in treating solid tumors, such as ovarian cancer.

“CAR T toxicity” is an early response to CAR T cell therapy and includes cytokine release syndrome (CRS) and neurotoxicity. Although CAR T toxicity is often considered an adverse reaction, it results from T cell activity and, thus, is also an indicator of likely efficacy of the CAR T cell therapy.

“Cytokine release syndrome” or “CRS” is characterized by high fever, myalgias, malaise, respiratory insufficiency, hemodynamic instability and capillary leak with hypotension, tachycardia, hypoxia, tachypnea, hemophagocytic lymphohistiocytosis/macrophage activation syndrome, or other organ toxicity associated with elevated serum cytokine concentrations. Elevated cytokines and associated molecules include interferon (IFN)-γ, IL-2, soluble IL-2Rα, IL-6, soluble IL-6R, granulocyte-macrophage colony-stimulating factor (GM-CSF), and other cytokines primarily secreted by the monocytes and/or macrophages such as IL-1, IL-6, IL-8, IL-10, IL-12, tumor necrosis factor (TNF)-α, IFN-α, monocyte chemotactic protein (MCP)-1, macrophage inflammatory protein (MIP) 1α. CRS usually occurs within a few days of administration of the genetically modified T cells to the patient.

“Neurotoxicity” associated with CAR T cell therapy is characterized by encephalopathy, headache, delirium, anxiety, tremor, aphasia, decreased level of consciousness, confusion, seizures or cerebral edema. Neurotoxicity can be associated with elevated serum concentrations of IL-6, IFN-7 and TNF-α.

An “effective amount” of a substance as that term is used herein is that amount sufficient to effect beneficial or desired results, including clinical results, and, as such, an “effective amount” depends upon the context in which it is being applied. In certain embodiments, an effective amount of a composition, e.g., a pharmaceutical composition comprising a therapeutic bacterium, described herein is an amount sufficient to improve clinical outcomes and/or promote cancer survival. For example, but not by way of limitation, an effective amount of a composition described herein, e.g., a pharmaceutical composition comprising a therapeutic bacterium, is an amount sufficient to increase the likelihood of cancer survival by at 10%, 20%, 30%, 40%, 50%, 60%, 70%, 80%, 90%, 95%, 98% or 99%. In certain embodiments, an effective amount of a composition described herein is an amount sufficient to decrease the likelihood of CAR T cell therapy associated toxicity. In certain embodiments, a decrease in the likelihood of CAR T cell therapy associated toxicity can be a 10%, 20%, 30%, 40%, 50%, 60%, 70%, 80%, 90%, 95%, 98% or 99% decrease. An effective amount can be administered in one or more administrations.

As used herein, and as well-understood in the art, “treatment” or administration of a “therapeutic agent” is an approach for obtaining beneficial or desired results, including clinical results. For purposes of this subject matter, beneficial or desired clinical results include, but are not limited to, increased likelihood of cancer survival following CAR T cell therapy; decreased likelihood of CAR T cell associated toxicity; alleviation or amelioration of one or more signs or symptoms; diminishment of extent of disease; stabilized (i.e., not worsening) state of disease; prevention of disease; delay or slowing of disease progression; remission of the disease (e.g., cancer remission); and/or amelioration or palliation of the disease state. The decrease can be a 10%, 20%, 30%, 40%, 50%, 60%, 70%, 80%, 90%, 95%, 98% or 99% decrease in severity of complications, e.g., toxicity, signs or symptoms. The increase can be a 10%, 20%, 30%, 40%, 50%, 60%, 70%, 80%, 90%, 95%, 98% or 99% increase in the length of cancer survival. “Treatment” can also mean prolonging cancer survival as compared to expected cancer survival if not receiving treatment. “Treatment” can also refer to increasing the likelihood of cancer survival and/or decreasing the likelihood of CAR T cell therapy associated toxicity.

The terms “prevent,” “preventing” and “prevention,” as used herein, refer to partially or completely delaying onset of one or more symptoms, features or clinical manifestations of a cancer; partially or completely delaying onset of one or more symptoms, features, or manifestations of a cancer (including, but not limited to, fatigue or reduced exercise capacity); partially or completely delaying progression from a cancer; partially or completely delaying CAR T cell associated toxicity and/or decreasing the risk of developing pathology associated with a cancer.

An “Operational Taxonomic Unit” or “OTU” are used herein to categorize bacteria based on sequence similarity, and are clusters of similar sequence variants of the 16S rRNA gene sequence. For example, but not by way of limitation, typically OTU clusters are defined by a 97% identity threshold to distinguish bacteria at the genus level.

The terms “cluster” or “cluster of related bacteria,” as used herein, can include two or more bacterial species or strains that are related by rRNA sequences, for example 16S rRNA gene (e.g., a variable region of the 16S rRNA gene sequence, such as V1, V2, V3, V4 or V5), similarity and/or evolutionary distance.

A “probiotic,” as used herein, is a microorganism or group of microorganisms that provides health benefits, or that is non-pathogenic, to a subject when consumed, ingested, or otherwise administered to a subject, for example, a reduction in the likelihood of relapse following cancer treatment. As used herein, the term probiotic can be used to describe, for example, probiotic bacteria and can include the bacteria described herein as well as other bacteria.

A “prebiotic,” as used herein, is a substance that promotes the growth, proliferation and/or survival of one or more bacteria or yeast. As used herein, the term prebiotic can be used to describe, for example, a nutritional supplement including plant fiber, or one or more of poorly-absorbed complex carbohydrates, oligosaccharides, inulin-type fructans or arabinoxylans.

A “postbiotic,” as used herein, is a substance derived from a probiotic organism. As used herein, the term postbiotic can be used to describe, for example, a protein expressed by one or more bacteria, a metabolic product of one or more bacteria, or media from a culture of one or more strains of bacteria.

As used herein, the term “microbiota diversity” refers to the number of and abundance distribution of distinct types of microbe organisms within a given body habitat and unless otherwise stated, it is measured in terms of Simpson reciprocal.

As used herein, the term “microbiota injury” refers to loss of diversity in the microbiota composition, or a composition that is different from baseline, or a composition that is different from that of a healthy person, or a composition that is dominated by a single taxon.

As used herein, the term “cancer survival” refers to the survival of a subject having a cancer for a certain amount of time (e.g., at least about 3 months, at least about 6 months, at least about 1 year, at least about 2 years, at least about 3 years, at least about 4 years, at least about 5 years, at least about 6 years, at least about 7 years, at least about 8 years, at least about 9 years, at least about 10 years, at least about 11 years, at least about 12 years, at least about 13 years, at least about 14 years, at least about 15 years or more) following a CAR T cell therapy. In certain embodiments, survival or cancer survival can be determined by measuring the proportion of subjects surviving during a 100 day period after a CAR T cell therapy.

As used herein, the term “recombinant cell” refers to cells which have some genetic modification from the original parent cells from which they are derived. Such cells can also be referred to as “genetically-engineered cells.” Such a genetic modification can be the result of an introduction of a heterologous gene (or nucleic acid) for expression of the gene product, e.g., a recombinant protein.

### II. Diagnostic Methods

In certain embodiments, the present disclosure provides for methods of determining whether a subject having a cancer has an increased likelihood or decreased likelihood to exhibit cancer survival following a CAR T cell therapy. In certain embodiments, the present disclosure provides for methods of determining whether a subject having a cancer has an increased likelihood or decreased likelihood to exhibit CAR T cell associated toxicity.

In certain embodiments, methods of the present disclosure include the analysis of the microbiome, e.g., a sample of the microbiome, of a subject. For example, but not by way of limitation, methods of the present disclosure include the analysis of a level of a bacterium or spores thereof in a sample of the subject compared to a reference level of the bacterium or spores thereof. In certain embodiments, methods of the present disclosure include the analysis of a level of a bacterial gene in a sample from the subject compared to a reference level of the bacterial gene.

In certain embodiments, a method for determining whether a subject having a cancer has an increased likelihood or decreased likelihood to exhibit cancer survival following CAR T cell therapy includes determining the level of a bacterium or spores thereof in a sample from the subject, comparing the level of the bacterium or spores thereof to a reference level, identifying the subject as having an increased likelihood of cancer survival following CAR T cell therapy based on the comparison, or identifying the subject as having a decreased likelihood of cancer survival following the CAR T cell therapy based on the comparison. In certain embodiments, the bacterium or spores thereof can be detected prior to treating the subject, for example, prior to a T cell therapy.

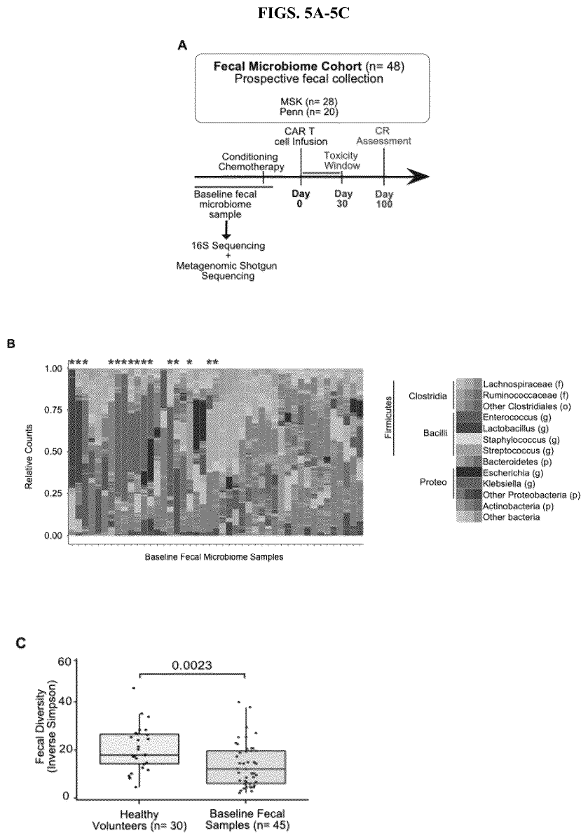

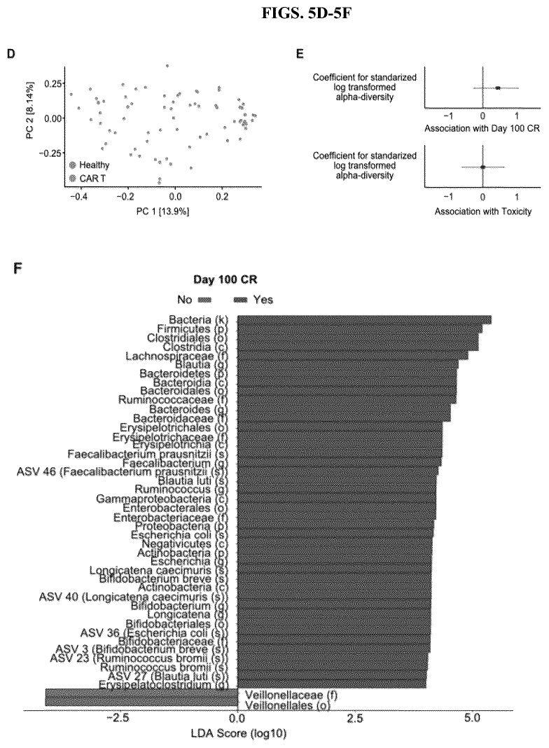

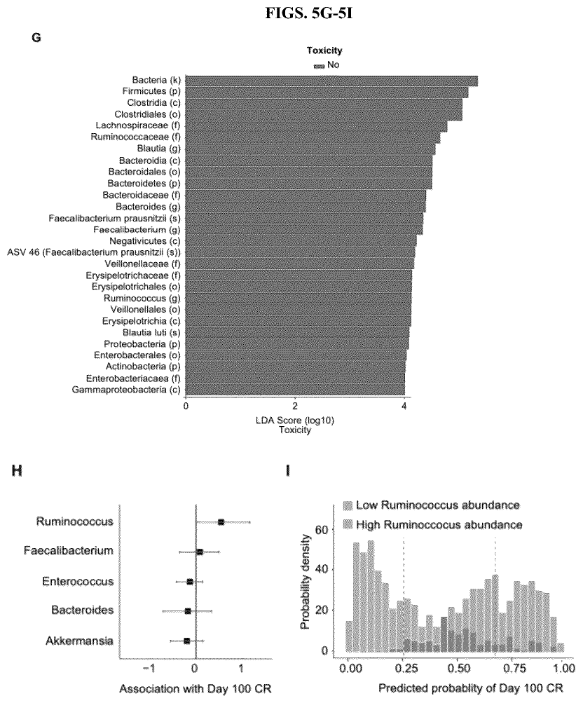

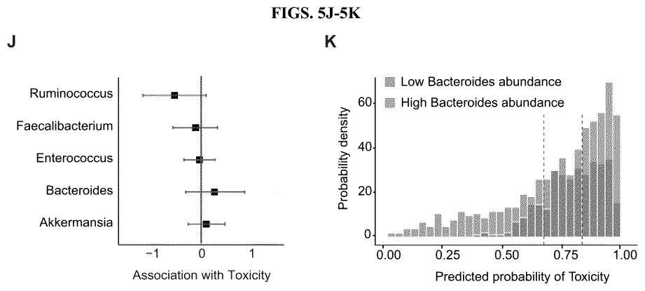

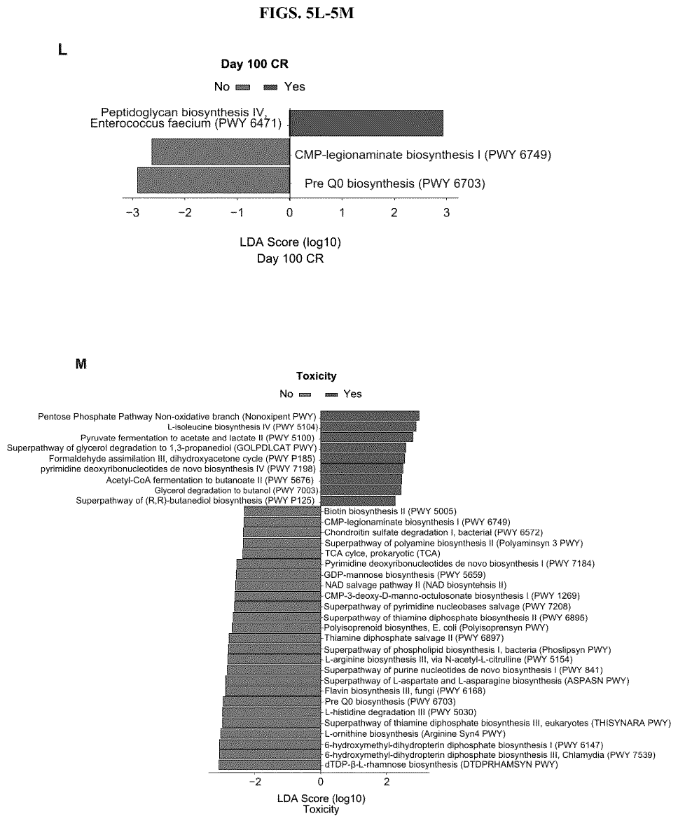

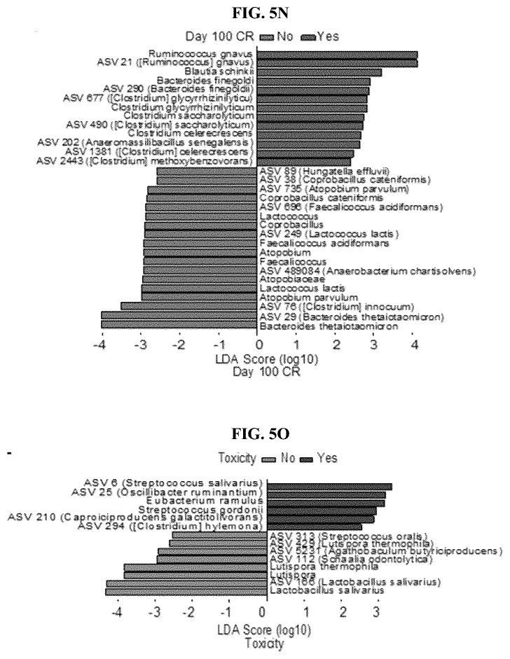

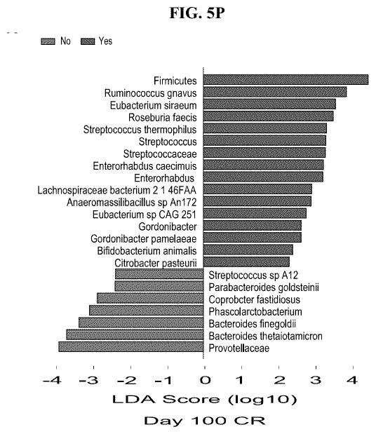

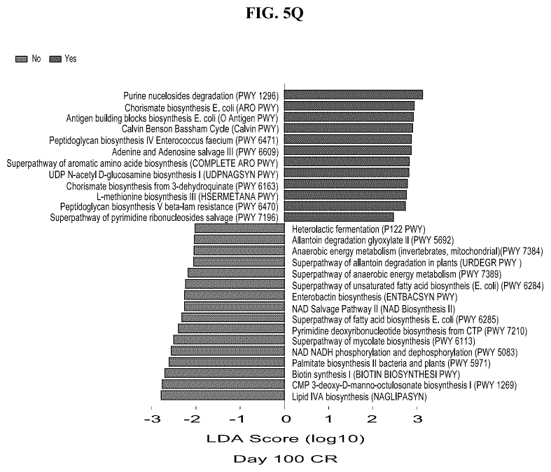

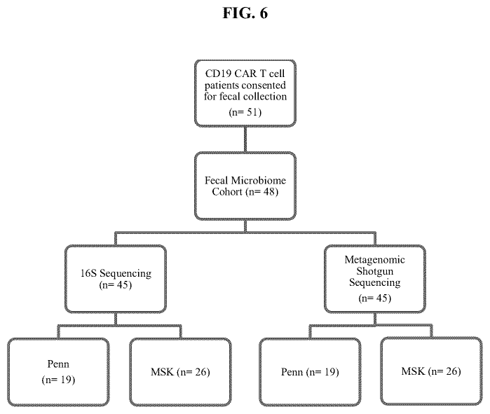

In certain embodiments, the bacterium analyzed in the sample of the subject for determining whether a subject having a cancer has an increased likelihood or decreased likelihood to exhibit cancer survival following CAR T cell therapy is provided in FIG. 5, e.g., FIGS. 5F, 5N and 5P. For example, but not by way of limitation, the bacterium or spore thereof is selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Hungatella effluvii, Coprobacillus cateniformis, Atopobium parvulum, Faecalicoccus acidiformans, Lactococcus lactis, Anaerobacterium chartisolvens, Clostridium innocuum, Bacteroides thetaiotamicron, Ruminococcus bromii, Bifidobacterium breve, Blautia luti, Longicatena caecimuris, Escherichia coli, Faecalibacterium prausnitzii and any combination thereof. In certain embodiments, the bacterium or spore thereof comprises a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Hungatella effluvii, Coprobacillus cateniformis, Atopobium parvulum, Faecalicoccus acidiformans, Lactococcus lactis, Anaerobacterium chartisolvens, Clostridium innocuum, Bacteroides thetaiotamicron, Ruminococcus bromii, Bifidobacterium breve, Blautia luti, Longicatena caecimuris, Escherichia coli, Faecalibacterium prausnitzii and or combination thereof.

In certain embodiments, the bacterium analyzed in the sample of the subject for determining whether a subject having a cancer has an increased likelihood or decreased likelihood to exhibit cancer survival following CAR T cell therapy is provided in FIG. 5, e.g., FIGS. 5F, 5N and 5P. For example, but not by way of limitation, the bacterium or spore thereof is selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Hungatella effluvii, Coprobacillus cateniformis, Atopobium parvulum, Faecalicoccus acidiformans, Lactococcus lactis, Anaerobacterium chartisolvens, Clostridium innocuum, Bacteroides thetaiotamicron and any combination thereof. In certain embodiments, the bacterium or spore thereof comprises a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Hungatella effluvii, Coprobacillus cateniformis, Atopobium parvulum, Faecalicoccus acidiformans, Lactococcus lactis, Anaerobacterium chartisolvens, Clostridium innocuum and Bacteroides thetaiotamicron or any combination thereof.

In certain embodiments, the bacterium analyzed in the sample of the subject for determining whether a subject having a cancer has an increased likelihood or decreased likelihood to exhibit cancer survival following CAR T cell therapy is provided in FIG. 5, e.g., FIGS. 5F, 5N and 5P. For example, but not by way of limitation, the bacterium or spore thereof is selected from the group consisting of Ruminococcus gnavus, Eubacterium siraeum, Roseburia faecis, Streptococcus thermophilus, Enterorhabdus caecimuris, Lachnospiraceae bacterium 2 1 46FAA, Anaeromassilibacillus sp An172, Eubacterium sp CAG 251, Gordonibacter pamelaeae, Bifidobacterium animalis, Citrobacter pasteurii, Streptococcus sp A12, Parabacteroides goldsteinii, Coprobacter fastidiosus, Bacteroides finegoldii, Bacteroides thetaiotamicron and any combination thereof. In certain embodiments, the bacterium or spore thereof comprises a 16S rRNA gene sequence having at least 94.5%, 95%, 97%, 98%, 98.7% or 99% sequence identity to the 16S rRNA gene sequence of Ruminococcus gnavus, Eubacterium siraeum, Roseburia faecis, Streptococcus thermophilus, Enterorhabdus caecimuris, Lachnospiraceae bacterium 2 1 46FAA, Anaeromassilibacillus sp An172, Eubacterium sp CAG 251, Gordonibacter pamelaeae, Bifidobacterium animalis, Citrobacter pasteurii, Streptococcus sp A12, Parabacteroides goldsteinii, Coprobacter fastidiosus, Bacteroides finegoldii, Bacteroides thetaiotamicron or any combination thereof.

In certain embodiments, the bacterium analyzed in the sample of the subject for determining whether a subject having a cancer has an increased likelihood or decreased likelihood to exhibit cancer survival following CAR T cell therapy is provided in FIG. 5, e.g., FIGS. 5F, 5N and 5P. For example, but not by way of limitation, the bacterium or spore thereof is selected from the group consisting of Ruminococcus bromii, Bifidobacterium breve, Blautia luti, Longicatena caecimuris, Escherichia coli, Faecalibacterium prausnitzii and any combination thereof. In certain embodiments, the bacterium or spore thereof comprises a 16S rRNA gene sequence having at least 94.5%, 95%, 97%, 98%, 98.7% or 99% sequence identity to the 16S rRNA gene sequence of Ruminococcus bromii, Bifidobacterium breve, Blautia luti, Longicatena caecimuris, Escherichia coli, Faecalibacterium prausnitzii or any combination thereof.

In certain embodiments, the bacterium or spore thereof is a species from the family Lachnospiraceae, Ruminococcaceae, Bacteroidaceae, Erysipelotrichaceae, Enterobacteriaceae, Bifidobacteriaceae and/or Veillonellaceae. In certain embodiments, the bacterium or spore thereof comprises a 16S rRNA gene sequence having at least 94.5%, 95%, 97%, 98%, 98.7% or 99% sequence identity to the 16S rRNA gene sequence of a bacterium or spore thereof that is a species from the family Lachnospiraceae, Ruminococcaceae, Bacteroidaceae, Erysipelotrichaceae, Enterobacteriaceae, Bifidobacteriaceae and/or Veillonellaceae.

In certain embodiments, the bacterium or spore thereof is a species from the genus Lactococcus, Coprobacillus, Atopobiaceae, Atopobium and/or Faecalicoccus. In certain embodiments, the bacterium or spore thereof comprises a 16S rRNA gene sequence having at least 94.5%, 95%, 97%, 98%, 98.7% or 99% sequence identity to the 16S rRNA gene sequence of a bacterium or spore thereof that is a species from the genus Lactococcus, Coprobacillus, Atopobiaceae, Atopobium and/or Faecalicoccus.

In certain embodiments, the bacterium or spore thereof is a species from the genus Blautia, Bacteroides, Ruminococcus, Bifidobacterium, Erysipelatoclostridium, Longicatena, Escherichia and/or Faecalibacterium. In certain embodiments, the bacterium or spore thereof comprises a 16S rRNA gene sequence having at least 94.5%, 95%, 97%, 98%, 98.7% or 99% sequence identity to the 16S rRNA gene sequence of a bacterium or spore thereof that is a species from the genus Blautia, Bacteroides, Ruminococcus, Bifidobacterium, Erysipelatoclostridium, Longicatena, Escherichia and/or Faecalibacterium.

In certain embodiments, the bacterium analyzed in the sample of the subject for determining whether a subject having a cancer has an increased likelihood or decreased likelihood to exhibit cancer survival following CAR T cell therapy is selected from the group consisting of Faecalibacterium prausnitzii, Blautia luti, Escherichia coli, Longicatena caecimuris, Bifidobacterium breve and Ruminococcus bromii. In certain embodiments, the bacterium or spore thereof comprises a 16S rRNA gene sequence having at least 94.5%, 95%, 97%, 98%, 98.7% or 99% sequence identity to the 16S rRNA gene sequence of Faecalibacterium prausnitzii, Blautia luti, Escherichia coli, Longicatena caecimuris, Bifidobacterium breve and/or Ruminococcus bromii.

In certain embodiments, the bacterium for determining whether a subject has an increased likelihood or decreased likelihood to exhibit cancer survival is of the Ruminococcacea family. In certain embodiments, the bacterium for determining whether a subject has an increased likelihood or decreased likelihood to exhibit cancer survival is of the Ruminococcus genus, e.g., Ruminococcus gnavus or Ruminococcus bromii. In certain non-limiting embodiments, the bacterium can comprise one or more strains of the bacterial species Ruminococcus gnavus, for example, found within the taxonomic group NCBI:txid33038, or a bacterium comprising a 16S rRNA gene sequence that has at least about 94.5%, 95%, 97%, 98%, 98.7% or 99% sequence identity with a 16S rRNA gene sequence of Ruminococcus gnavus. In certain non-limiting embodiments, the bacterium can comprise one or more strains of the bacterial species Ruminococcus bromii, for example, found within the taxonomic group NCBI:txid 40518, or a bacterium comprising a 16S rRNA gene sequence that has at least about 94.5%, 95%, 97%, 98%, 98.7% or 99% sequence identity with a 16S rRNA gene sequence of Ruminococcus bromii.

In certain embodiments, the bacterium for determining whether a subject has an increased likelihood or decreased likelihood to exhibit cancer survival is of the Blautia genus, e.g., Blautia schinkii or Blautia luti. In certain non-limiting embodiments, the bacterium can comprise one or more strains of the bacterial species Blautia schinkii, for example, found within the taxonomic group NCBI:txid180164, or a bacterium comprising a 16S rRNA gene sequence that has at least about 94.5%, 95%, 97%, 98%, 98.7% or 99% sequence identity with a 16S rRNA gene sequence of Blautia schinkii. In certain non-limiting embodiments, the bacterium can comprise one or more strains of the bacterial species Blautia luti, for example, found within the taxonomic group NCBI:txid89014, or a bacterium comprising a 16S rRNA gene sequence that has at least about 94.5%, 95%, 97%, 98%, 98.7% or 99% sequence identity with a 16S rRNA gene sequence of Blautia luti.

In certain embodiments, the bacterium for determining whether a subject has an increased likelihood or decreased likelihood to exhibit cancer survival is of the Bacteroidaceae family. In certain embodiments, the bacterium for determining whether a subject has an increased likelihood or decreased likelihood to exhibit cancer survival is of the Bacteroides genus, e.g., Bacteroides finegoldii. In certain non-limiting embodiments, the bacterium can comprise one or more strains of the bacterial species Bacteroides finegoldii, for example, found within the taxonomic group NCBI:txid338188, or a bacterium comprising a 16S rRNA gene sequence that has at least about 94.5%, 95%, 97%, 98%, 98.7% or 99% sequence identity with a 16S rRNA gene sequence of Bacteroides finegoldii.

In certain embodiments, the bacterium for determining whether a subject has an increased likelihood or decreased likelihood to exhibit cancer survival is Clostridium glycyrrhizinilyticum. In certain non-limiting embodiments, the bacterium can comprise one or more strains of the bacterial species Clostridium glycyrrhizinilyticum, for example, found within the taxonomic group NCBI:txid342942, or a bacterium comprising a 16S rRNA gene sequence that has at least about 94.5%, 95%, 97%, 98%, 98.7% or 99% sequence identity with a 16S rRNA gene sequence of Clostridium glycyrrhizinilyticum.

In certain embodiments, the bacterium for determining whether a subject has an increased likelihood or decreased likelihood to exhibit cancer survival is Clostridium saccharolyticum. In certain non-limiting embodiments, the bacterium can comprise one or more strains of the bacterial species Clostridium saccharolyticum, for example, found within the taxonomic group NCBI:txid84030, or a bacterium comprising a 16S rRNA gene sequence that has at least about 94.5%, 95%, 97%, 98%, 98.7% or 99% sequence identity with a 16S rRNA gene sequence of Clostridium saccharolyticum.

In certain embodiments, the bacterium for determining whether a subject has an increased likelihood or decreased likelihood to exhibit cancer survival is Clostridium celerecrescens. In certain non-limiting embodiments, the bacterium can comprise one or more strains of the bacterial species Clostridium celerecrescens, for example, found within the taxonomic group NCBI:txid29354, or a bacterium comprising a 16S rRNA gene sequence that has at least about 94.5%, 95%, 97%, 98%, 98.7% or 99% sequence identity with a 16S rRNA gene sequence of Clostridium celerecrescens.

In certain embodiments, the bacterium for determining whether a subject has an increased likelihood or decreased likelihood to exhibit cancer survival is Anaeromassilibacillus senegalensis. In certain non-limiting embodiments, the bacterium can comprise one or more strains of the bacterial species Anaeromassilibacillus senegalensis, for example, found within the taxonomic group NCBI:txid1673717, or a bacterium comprising a 16S rRNA gene sequence that has at least about 94.5%, 95%, 97%, 98%, 98.7% or 99% sequence identity with a 16S rRNA gene sequence of Anaeromassilibacillus senegalensis.

In certain embodiments, the bacterium for determining whether a subject has an increased likelihood or decreased likelihood to exhibit cancer survival is Clostridium methoxybenzovorans. In certain non-limiting embodiments, the bacterium can comprise one or more strains of the bacterial species Clostridium methoxybenzovorans, for example, found within the taxonomic group NCBI:txid81424, or a bacterium comprising a 16S rRNA gene sequence that has at least about 94.5%, 95%, 97%, 98%, 98.7% or 99% sequence identity with a 16S rRNA gene sequence of Clostridium methoxybenzovorans.

In certain embodiments, the bacterium for determining whether a subject has an increased likelihood or decreased likelihood to exhibit cancer survival is Hungatella effluvii. In certain non-limiting embodiments, the bacterium can comprise one or more strains of the bacterial species Hungatella effluvii, for example, found within the taxonomic group NCBI:txid1096246, or a bacterium comprising a 16S rRNA gene sequence that has at least about 94.5%, 95%, 97%, 98%, 98.7% or 99% sequence identity with a 16S rRNA gene sequence of Hungatella effluvii.

In certain embodiments, the bacterium for determining whether a subject has an increased likelihood or decreased likelihood to exhibit cancer survival is of the Coprobacillus genus, e.g., Coprobacillus cateniformis. In certain non-limiting embodiments, the bacterium can comprise one or more strains of the bacterial species Coprobacillus cateniformis, for example, found within the taxonomic group NCBI:txid100884, or a bacterium comprising a 16S rRNA gene sequence that has at least about 94.5%, 95%, 97%, 98%, 98.7% or 99% sequence identity with a 16S rRNA gene sequence of Coprobacillus cateniformis.

In certain embodiments, the bacterium for determining whether a subject has an increased likelihood or decreased likelihood to exhibit cancer survival is of the Atopobium genus, e.g., Atopobium parvulum. In certain non-limiting embodiments, the bacterium can comprise one or more strains of the bacterial species Atopobium parvulum, for example, found within the taxonomic group NCBI:txid1382, or a bacterium comprising a 16S rRNA gene sequence that has at least about 94.5%, 95%, 97%, 98%, 98.7% or 99% sequence identity with a 16S rRNA gene sequence of Atopobium parvulum.

In certain embodiments, the bacterium for determining whether a subject has an increased likelihood or decreased likelihood to exhibit cancer survival is of the Lactococcus genus, e.g., Lactococcus lactis. In certain non-limiting embodiments, the bacterium can comprise one or more strains of the bacterial species Lactococcus lactis, for example, found within the taxonomic group NCBI:txid1358, or a bacterium comprising a 16S rRNA gene sequence that has at least about 94.5%, 95%, 97%, 98%, 98.7% or 99% sequence identity with a 16S rRNA gene sequence of Lactococcus lactis.

In certain embodiments, the bacterium for determining whether a subject has an increased likelihood or decreased likelihood to exhibit cancer survival is of the Faecalicoccus genus, e.g., Faecalicoccus acidiformans. In certain non-limiting embodiments, the bacterium can comprise one or more strains of the bacterial species Faecalicoccus acidiformans, for example, found within the taxonomic group NCBI:txid915173, or a bacterium comprising a 16S rRNA gene sequence that has at least about 94.5%, 95%, 97%, 98%, 98.7% or 99% sequence identity with a 16S rRNA gene sequence of Faecalicoccus acidiformans.

In certain embodiments, the bacterium for determining whether a subject has an increased likelihood or decreased likelihood to exhibit cancer survival is of the Faecalibacterium genus, e.g., Faecalibacterium prausnitzii. In certain non-limiting embodiments, the bacterium can comprise one or more strains of the bacterial species Faecalibacterium prausnitzii, for example, found within the taxonomic group NCBI:txid853, or a bacterium comprising a 16S rRNA gene sequence that has at least about 94.5%, 95%, 97%, 98%, 98.7% or 99% sequence identity with a 16S rRNA gene sequence of Faecalibacterium prausnitzii.

In certain embodiments, the bacterium for determining whether a subject has an increased likelihood or decreased likelihood to exhibit cancer survival is of the Longicatena genus, e.g., Longicatena caecimuris. In certain non-limiting embodiments, the bacterium can comprise one or more strains of the bacterial species Longicatena caecimuris, for example, found within the taxonomic group NCBI:txid1796635, or a bacterium comprising a 16S rRNA gene sequence that has at least about 94.5%, 95%, 97%, 98%, 98.7% or 99% sequence identity with a 16S rRNA gene sequence of Longicatena caecimuris.

In certain embodiments, the bacterium for determining whether a subject has an increased likelihood or decreased likelihood to exhibit cancer survival is of the Bifidobacteriacea family. In certain embodiments, the bacterium for determining whether a subject has an increased likelihood or decreased likelihood to exhibit cancer survival is of the Bifidobacterium genus, e.g., Bifidobacterium breve. In certain non-limiting embodiments, the bacterium can comprise one or more strains of the bacterial species Bifidobacterium breve, for example, found within the taxonomic group NCBI:txid1685, or a bacterium comprising a 16S rRNA gene sequence that has at least about 94.5%, 95%, 97%, 98%, 98.7% or 99% sequence identity with a 16S rRNA gene sequence of Bifidobacterium breve.

In certain embodiments, the bacterium for determining whether a subject has an increased likelihood or decreased likelihood to exhibit cancer survival is of the Escherichia genus, e.g., Escherichia coli. In certain non-limiting embodiments, the bacterium can comprise one or more strains of the bacterial species Escherichia coli, for example, found within the taxonomic group NCBI:txid562, or a bacterium comprising a 16S rRNA gene sequence that has at least about 94.5%, 95%, 97%, 98%, 98.7% or 99% sequence identity with a 16S rRNA gene sequence of Escherichia coli.

In certain embodiments, the bacterium for determining whether a subject has an increased likelihood or decreased likelihood to exhibit cancer survival is Anaerobacterium chartisolvens. In certain non-limiting embodiments, the bacterium can comprise one or more strains of the bacterial species Anaerobacterium chartisolvens, for example, found within the taxonomic group NCBI:txid1297424, or a bacterium comprising a 16S rRNA gene sequence that has at least about 94.5%, 95%, 97%, 98%, 98.7% or 99% sequence identity with a 16S rRNA gene sequence of Anaerobacterium chartisolvens.

In certain embodiments, the bacterium for determining whether a subject has an increased likelihood or decreased likelihood to exhibit cancer survival is of the Atopobiaceae genus. In certain non-limiting embodiments, the one or more bacteria can comprise one or more species from the bacteria genus Atopobiaceae, for example, found within the taxonomic group NCBI:txid 1643824.

In certain embodiments, the bacterium for determining whether a subject has an increased likelihood or decreased likelihood to exhibit cancer survival is of the Erysipelotrichaceae family. In certain embodiments, the bacterium for determining whether a subject has an increased likelihood or decreased likelihood to exhibit cancer survival is of the Erysipelatoclostridium genus. In certain non-limiting embodiments, the one or more bacteria can comprise one or more species from the bacteria genus Erysipelatoclostridium, for example, found within the taxonomic group NCBI:txid1505663.

In certain embodiments, the bacterium for determining whether a subject has an increased likelihood or decreased likelihood to exhibit cancer survival is Clostridium innocuum. In certain non-limiting embodiments, the bacterium can comprise one or more strains of the bacterial species Clostridium innocuum, for example, found within the taxonomic group NCBI:txid1522, or a bacterium comprising a 16S rRNA gene sequence that has at least about 94.5%, 95%, 97%, 98%, 98.7% or 99% sequence identity with a 16S rRNA gene sequence of Clostridium innocuum.

In certain embodiments, the bacterium for determining whether a subject has an increased likelihood or decreased likelihood to exhibit cancer survival is of the Veillonellaceae family. In certain non-limiting embodiments, the one or more bacteria can comprise one or more species from the bacteria family Veillonellaceae, for example, found within the taxonomic group NCBI:txid31977.

In certain embodiments, the bacterium for determining whether a subject has an increased likelihood or decreased likelihood to exhibit cancer survival is of the Bacteroides genus, e.g., Bacteroides thetaiotamicron. In certain non-limiting embodiments, the bacterium can comprise one or more strains of the bacterial species Bacteroides thetaiotaomicron, for example, found within the taxonomic group NCBI:txid818, or a bacterium comprising a 16S rRNA gene sequence that has at least about 94.5%, 95%, 97%, 98%, 98.7% or 99% sequence identity with a 16S rRNA gene sequence of Bacteroides thetaiotaomicron.

In certain embodiments, the bacterium for determining whether a subject has an increased likelihood or decreased likelihood to exhibit cancer survival is of the Enterobacteriaceae family. In certain non-limiting embodiments, the bacterium comprises a 16S rRNA gene sequence that has at least about 94.5%, 95%, 97%, 98%, 98.7% or 99% sequence identity with a 16S rRNA gene sequence of bacterium of the Enterobacteriaceae family.

In certain embodiments, the bacterium for determining whether a subject has an increased likelihood or decreased likelihood to exhibit cancer survival is of the Lachnospiraceae family. In certain non-limiting embodiments, the bacterium comprises a 16S rRNA gene sequence that has at least about 94.5%, 95%, 97%, 98%, 98.7% or 99% sequence identity with a 16S rRNA gene sequence of bacterium of the Lachnospiraceae family.

In certain embodiments, a method of the present disclosure includes identifying a subject as having an increased likelihood of cancer survival if the level of a bacterium identified as being associated with increased day 100 CR in FIG. 5, e.g., FIGS. 5F, 5N and 5P, e.g., Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Faecalibacterium prausnitzii, Blautia luti, Escherichia coli, Longicatena caecimuris, Bifidobacterium breve and/or Ruminococcus bromii, is higher in the subject's sample than a reference bacterium level. In certain embodiments, a method of the present disclosure includes identifying a subject as having an increased likelihood of cancer survival if the level of a bacterium identified as being associated with increased day 100 CR in FIG. 5, e.g., FIGS. 5F, 5N and 5P, e.g., or a bacterium comprising a 16S rRNA gene sequence that has at least about 94.5%, 95%, 97%, 98%, 98.7% or 99% sequence identity with a 16S rRNA gene of a bacterium identified as being associated with increased day 100 CR in FIG. 5, e.g., FIGS. 5F, 5N and 5P, Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Faecalibacterium prausnitzii, Blautia luti, Escherichia coli, Longicatena caecimuris, Bifidobacterium breve and/or Ruminococcus bromii, is higher in the subject's sample than a reference bacterium level.

In certain embodiments, a method of the present disclosure includes identifying a subject as having an increased likelihood of cancer survival if the level of a bacterium identified as being associated with increased day 100 CR in FIG. 5, e.g., FIGS. 5F, 5N and 5P, e.g., Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis and/or Clostridium methoxybenzovorans, is higher in the subject's sample than a reference bacterium level. For example, but not by way of limitation, a subject is identified as having an increased likelihood of cancer survival if the level of Ruminococcus gnavus or spores thereof is higher in the subject's sample than a reference bacterium level. In certain embodiments, the reference bacterium level is the level of the bacterium or spores thereof from a subject that has been treated with a CAR T cell therapy and exhibited cancer survival.

In certain embodiments, a method of the present disclosure includes identifying a subject as having an increased likelihood of cancer survival if the level of a bacterium identified as being associated with increased day 100 CR in FIG. 5, e.g., FIGS. 5F, 5N and 5P, e.g., Faecalibacterium prausnitzii, Blautia luti, Escherichia coli, Longicatena caecimuris, Bifidobacterium breve and/or Ruminococcus bromii, is higher in the subject's sample than a reference bacterium level. For example, but not by way of limitation, a subject is identified as having an increased likelihood of cancer survival if the level of Faecalibacterium prausnitzii or spores thereof is higher in the subject's sample than a reference bacterium level. In certain embodiments, the reference bacterium level is the level of the bacterium or spores thereof from a subject that has been treated with a CAR T cell therapy and exhibited cancer survival.

In certain embodiments, a method of the present disclosure includes identifying a subject as having a decreased likelihood of cancer survival if the level of a bacterium identified as being associated with decreased day 100 CR in FIG. 5, e.g., FIGS. 5F, 5N and 5P, or a bacterium comprising a 16S rRNA gene sequence that has at least about 94.5%, 95%, 97%, 98%, 98.7% or 99% sequence identity with a 16S rRNA gene of a bacterium identified as being associated with decreased day 100 CR in FIG. 5, e.g., FIGS. 5F, 5N and 5P, is higher in the subject's sample than a reference bacterium level. In certain embodiments, a method of the present disclosure includes identifying a subject as having a decreased likelihood of cancer survival if the level of a bacterium identified as being associated with decreased day 100 CR in FIG. 5, e.g., FIGS. 5F, 5N and 5P, e.g., Hungatella effluvii, Coprobacillus cateniformis, Atopobium parvulum, Faecalicoccus acidiformans, Coprobacillus, Lactococcus lactis, Anaerobacterium chartisolvens, Clostridium innocuum and/or Bacteroides thetaiotaomicron, is higher in the subject's sample than a reference bacterium level. For example, but not by way of limitation, a subject is identified as having a decreased likelihood of cancer survival if the level of Bacteroides thetaiotaomicron or spores thereof is higher in the subject's sample than a reference bacterium level. In certain embodiments, the reference bacterium level is the level of the bacterium or spores thereof from a subject that has been treated with a CAR T cell therapy and did not exhibit cancer survival.

In certain embodiments, a method of the present disclosure includes identifying a subject as having a decreased likelihood of cancer survival if the level of a bacterial species of a genus identified as being associated with decreased day 100 CR in FIG. 5, e.g., FIGS. 5F, 5N and 5P, or a bacterium comprising a 16S rRNA gene sequence that has at least about 94.5%, 95%, 97%, 98%, 98.7% or 99% sequence identity with a 16S rRNA gene of a bacterium of a genus identified as being associated with decreased day 100 CR in FIG. 5, e.g., FIGS. 5F, 5N and 5P, is higher in the subject's sample than a reference bacterium level. In certain embodiments, a method of the present disclosure includes identifying a subject as having a decreased likelihood of cancer survival if the level of a bacterial species of a genus identified as being associated with decreased day 100 CR in FIG. 5, e.g., FIGS. 5F, 5N and 5P, e.g., Lactococcus, Coprobacillus, Atopobiaceae, Atopobium and/or Faecalicoccus, is higher in the subject's sample than a reference bacterium level.

In certain embodiments, a method of the present disclosure includes identifying a subject as having a decreased likelihood of cancer survival if the level of a bacterial species of a family identified as being associated with decreased day 100 CR in FIG. 5, e.g., FIGS. 5F, 5N and 5P, e.g., the family Veillonellaceae, is higher in the subject's sample than a reference bacterium level.

In certain embodiments, cancer survival refers to the survival of the subject at least about 100 days following a cancer treatment (e.g., a CAR T cell therapy). In certain embodiments, cancer survival refers to the survival of the subject at least 3 months, at least 6 months, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years or more following a cancer treatment. In certain embodiments, cancer survival refers to the survival of the subject at least about 100 days following a cancer treatment, e.g., a CAR T cell therapy.

In certain embodiments, a method for determining whether a subject having a cancer has an increased likelihood to exhibit cancer survival following a CAR T cell therapy includes determining the level of a bacterial gene in a sample from the subject, comparing the level of the bacterial gene to a reference level of the bacterial gene, identifying the subject as having an increased likelihood to exhibit cancer survival following CAR T cell therapy based on the comparison.

In certain embodiments, non-limiting examples of the bacterial genes associated with a likelihood of cancer survival are provided in FIG. 5, e.g., FIGS. 5L and 5Q. In certain embodiments, the bacterial gene determined in the sample of the subject is selected from the group consisting of the genes involved in purine nucleosides degradation (e.g., PWY 1296), chorismate biosynthesis (e.g., ARO PWY), antigen building blocks biosynthesis (e.g., O Antigen Pathway), Calvin Benson Bassham cycle (e.g., Calvin PWY), peptidoglycan biosynthesis IV Enterococcus faecium (e.g., PWY 6471), adenine and adenosine salvage III (e.g., PWY 6609), superpathway of aromatic amino acid biosynthesis (e.g., COMPLETE ARO PWY), UDP N-acetyl D glucosamine biosynthesis (e.g., UDPNAGSYN PWY), chorismate biosynthesis from 3-dehydroguinate (e.g., PWY 6163), L-methionine biosynthesis III (e.g., HSERMETANA PWY), peptidoglycan biosynthesis V beta-lactam resistance (e.g., PWY 6470), superpathway of pyrimidine ribonucleosides salvage (e.g., PWY 7196), heterolactic fermentation (e.g., P122 PWY), allantoin degradation glyoxylate II (e.g., PWY 5692), anaerobic energy metabolism (e.g., PWY 7384), superpathway of allantoin degradation (e.g., URDEGR PWY), superpathway of anaerobic energy metabolism (e.g., PWY 7389), superpathway of unsaturated fatty acid biosynthesis (e.g., PWY 6284), enterobactin biosynthesis (e.g., ENTBACSYN PWY), NAD salvage pathway III (e.g., NAD Biosynthesis II), superpathway of fatty acid biosynthesis (e.g., PWY 6285), pyrimidine deoxyribonucleotide biosynthesis from CTP (e.g., PWY 7210), superpathway of mycolate biosynthesis (e.g., PWY 6113), NAD NADH phosphorylation and dephosphorylation (e.g., PWY 5083), palmitate biosynthesis II bacteria and plants (e.g., PWY 5971), biotin synthesis I (e.g., BIOTIN BIOSYNTHESIS PWY), CMP 3-deoxy-D-manno-octulosonate biosynthesis (e.g., PWY 1269), lipid IVA biosynthesis (e.g., NAGLIPASYN), CMP-legionaminate biosynthesis (e.g., PWY 6749) and/or Pre QO biosynthesis (e.g., PWY 6703). In certain embodiments, the bacterial gene determined in the sample of the subject is selected from the group consisting of the genes involved in aromatic amino acid biosynthesis (e.g., chorismate biosynthesis E. coli and the superpathway of aromatic amino acid biosynthesis (e.g., COMPLETE ARO PWY)) and/or peptidoglycan biosynthesis (peptidoglycan biosynthesis IV Enterococcus faecium (e.g., PWY 6471) and peptidoglycan biosynthesis V beta-lactam resistance (e.g., PWY 6470)). In certain embodiments, the bacterial gene determined in the sample of the subject is a gene involved in peptidoglycan biosynthesis (peptidoglycan biosynthesis IV Enterococcus faecium (e.g., PWY 6471)). In certain embodiments, the bacterial gene determined in the sample of the subject is a gene involved in CMP-legionaminate biosynthesis (e.g., PWY 6749) and/or Pre QO biosynthesis (e.g., PWY 6703).

In certain embodiments, a subject is identified as having an increased likelihood of cancer survival if the level of a bacterial gene identified as being associated with increased day 100 CR in FIG. 5, e.g., FIGS. 5L and 5Q, e.g., a bacterial gene involved in purine nucleosides degradation (e.g., PWY 1296), chorismate biosynthesis (e.g., ARO PWY), antigen building blocks biosynthesis (e.g., O Antigen Pathway), Calvin Benson Bassham cycle (e.g., Calvin PWY), peptidoglycan biosynthesis IV (e.g., PWY 6471), adenine and adenosine salvage III (e.g., PWY 6609), superpathway of aromatic amino acid biosynthesis (e.g., COMPLETE ARO PWY), UDP N-acetyl D glucosamine biosynthesis (e.g., UDPNAGSYN PWY), chorismate biosynthesis from 3-dehydroguinate (e.g., PWY 6163), L-methionine biosynthesis III (e.g., HSERMETANA PWY), peptidoglycan biosynthesis V beta-lactam resistance (e.g., PWY 6470) and/or superpathway of pyrimidine ribonucleosides salvage (e.g., PWY 7196), is higher in the subject's sample than the reference bacterial gene level. For example, but not by way of limitation, a subject is identified as having an increased likelihood of cancer survival if the level of one or more genes involved in aromatic amino acid biosynthesis (e.g., chorismate biosynthesis and the superpathway of aromatic amino acid biosynthesis (COMPLETE ARO PWY)), peptidoglycan biosynthesis (peptidoglycan biosynthesis IV (PWY 6471) and/or peptidoglycan biosynthesis V beta-lactam resistance (PWY 6470)) is higher in the subject's sample than the reference level of the bacterial genes. In certain embodiments, the reference level of the bacterial gene is the level of bacterial gene from a subject that has been treated with a CAR T cell therapy and exhibited cancer survival. In certain embodiments, a subject is identified as having an increased likelihood of cancer survival if the level of one or more genes involved in peptidoglycan biosynthesis (e.g., peptidoglycan biosynthesis IV (PWY 6471)) is higher in the subject's sample than the reference level of the bacterial genes.

In certain embodiments, a subject is identified as having a decreased likelihood of cancer survival if the level of a bacterial gene identified as being associated with decreased day 100 CR in FIG. 5, e.g., FIGS. 5L and 5Q, e.g., a bacterial gene involved in heterolactic fermentation (e.g., P122 PWY), allantoin degradation glyoxylate II (e.g., PWY 5692), anaerobic energy metabolism (PWY 7384), superpathway of allantoin degradation (e.g., URDEGR PWY), superpathway of anaerobic energy metabolism (e.g., PWY 7389), superpathway of unsaturated fatty acid biosynthesis (PWY 6284), enterobactin biosynthesis (e.g., ENTBACSYN PWY), NAD salvage pathway III (e.g., NAD Biosynthesis II), superpathway of fatty acid biosynthesis (e.g., PWY 6285), pyrimidine deoxyribonucleotide biosynthesis from CTP (e.g., PWY 7210), superpathway of mycolate biosynthesis (e.g., PWY 6113), NAD NADH phosphorylation and dephosphorylation (e.g., PWY 5083), palmitate biosynthesis II bacteria and plants (e.g., PWY 5971), biotin synthesis I (e.g., BIOTIN BIOSYNTHESIS PWY), CMP 3-deoxy-D-manno-octulosonate biosynthesis (e.g., PWY 1269), CMP-legionaminate biosynthesis (e.g., PWY 6749), Pre QO biosynthesis (e.g., PWY 6703) and/or lipid IVA biosynthesis (e.g., NAGLIPASYN), is higher in the subject's sample than the bacterial gene level. For example, but not by way of limitation, a subject is identified as having a decreased likelihood of cancer survival if the level of one or more genes involved in lipid IVA biosynthesis is higher in the subject's sample than the reference level of the bacterial genes. In certain embodiments, a subject is identified as having a decreased likelihood of cancer survival if the level of one or more genes involved in CMP-legionaminate biosynthesis (e.g., PWY 6749) and/or Pre QO biosynthesis (e.g., PWY 6703) is higher in the subject's sample than the reference level of the bacterial genes. In certain embodiments, the reference level of the bacterial gene is the level of bacterial gene from a subject that has been treated with a CAR T cell therapy and did not exhibit cancer survival.

In certain embodiments, a method for determining whether a subject having a cancer has a decreased likelihood or an increased likelihood to exhibit cancer survival following CAR T cell therapy includes determining whether the subject has been administered an antibiotic prior to CAR T cell therapy. In certain embodiments, the antibiotic is administered less than about 8 weeks, less than about 7 weeks, less than about 6 weeks, less than about 5 weeks, less than about 4 weeks, less than about 3 weeks, less than about 2 weeks, less than about 1 week prior to CAR T cell therapy. In certain embodiments, the antibiotic is administered less than about 4 weeks prior to CAR T cell therapy. In certain embodiments, the antibiotic is administered about 4 weeks prior to CAR T cell therapy.

In certain embodiments, a subject is identified as having a decreased likelihood of cancer survival following the CAR T cell therapy if the subject has been administered piperacillin-tazobactam, imipenem-cilastatin, meropenem or a combination thereof prior to CAR T cell therapy. In certain embodiments, a subject having a B-cell malignancy is identified as having a decreased likelihood of cancer survival following the CAR T cell therapy if the subject has been administered piperacillin-tazobactam, imipenem-cilastatin, meropenem or a combination thereof prior to CAR T cell therapy. In certain embodiments, a subject having NHL or ALL is identified as having a decreased likelihood of cancer survival following the CAR T cell therapy if the subject has been administered piperacillin-tazobactam, imipenem-cilastatin, meropenem or a combination thereof prior to CAR T cell therapy. In certain embodiments, a subject is identified as having a decreased likelihood of cancer survival following the CAR T cell therapy if the subject has been administered an antibiotic targeting obligate anaerobes prior to CAR T cell therapy.

The present disclosure further provides methods of identifying a subject as having an increased likelihood of a CAR T cell therapy toxicity or a decreased likelihood of a CAR T cell associated toxicity. For example, but not by way of limitation, the CAR T cell associated toxicity is a cytokine release syndrome or a neurotoxicity. In certain embodiments, a method of identifying a subject as having an increased likelihood of a CAR T cell associated toxicity or a decreased likelihood of a CAR T cell associated toxicity includes determining the level of a bacterium or spores thereof in a sample from the subject, comparing the level of the bacterium or spores thereof to a reference level, identifying the subject as having an increased or decreased likelihood of a CAR T cell associated toxicity based on the comparison.

In certain embodiments, the bacterium or spores thereof analyzed in the sample of the subject for determining whether the subject has an increased likelihood of a CAR T cell associated toxicity or a decreased likelihood of a CAR T cell associated toxicity is provided in FIG. 5, e.g., FIGS. 5G and 5O. For example, but not by way of limitation, the bacterium or spores thereof analyzed in the sample of the subject for determining whether the subject has an increased likelihood of a CAR T cell associated toxicity or a decreased likelihood of a CAR T cell associated toxicity is selected from the group consisting of Streptococcus salivarius, Oscillibacter ruminantium, Eubacterium ramulus, Streptococcus gordonii, Caproiciproducens galactitolivorans, Clostridium hylemonae, Streptococcus oralis, Lutispora thermophila, Agathobaculum butyriciproducens, Schaalia odontolytica, Lactobacillus salivarius and any combination thereof or a bacterium comprising a 16S rRNA gene sequence having at least 94.5%, 95%, 97%, 98%, 98.7% or 99% sequence identity to the 16S rRNA gene sequence of Streptococcus salivarius, Oscillibacter ruminantium, Eubacterium ramulus, Streptococcus gordonii, Caproiciproducens galactitolivorans, Clostridium hylemonae, Streptococcus oralis, Lutispora thermophila, Agathobaculum butyriciproducens, Schaalia odontolytica and/or Lactobacillus salivarius.

In certain embodiments, the bacterium for determining whether a subject has a decreased likelihood of a CAR T cell associated toxicity is from the genera Blautia, Ruminococcus, Bacteroides and/or Faecalibacterium or a bacterium comprising a 16S rRNA gene sequence having at least 94.5%, 95%, 97%, 98%, 98.7% or 99% sequence identity to the 16S rRNA gene sequence of a bacterium from the genera Blautia, Ruminococcus, Bacteroides and/or Faecalibacterium. In certain embodiments, the bacterium for determining whether a subject has a decreased likelihood of a CAR T cell associated toxicity is Faecalibacterium prausnitzii and/or Blautia luti or a bacterium comprising a 16S rRNA gene sequence having at least 94.5%, 95%, 97%, 98%, 98.7% or 99% sequence identity to the 16S rRNA gene sequence of Faecalibacterium prausnitzii and/or Blautia luti.

In certain embodiments, the bacterium for determining whether a subject has a decreased likelihood of a CAR T cell associated toxicity is from the families Lachnospiraceae, Ruminococcacea, Bacteroidaceae, Veillonellaceae, Erysipelotrichaceae and/or Enterobacteriaceae or a bacterium comprising a 16S rRNA gene sequence having at least 94.5%, 95%, 97%, 98%, 98.7% or 99% sequence identity to the 16S rRNA gene sequence of a bacterium from the families Lachnospiraceae, Ruminococcacea, Bacteroidaceae, Veillonellaceae, Erysipelotrichaceae and/or Enterobacteriaceae.

In certain embodiments, the bacterium for determining whether the subject has an increased likelihood of a CAR T cell associated toxicity or a decreased likelihood of a CAR T cell associated toxicity is Streptococcus salivarius. In certain non-limiting embodiments, the bacterium can comprise one or more strains of the bacterial species Streptococcus salivarius, for example, found within the taxonomic group NCBI:txid1304, or a bacterium comprising a 16S rRNA gene sequence that has at least about 94.5%, 95%, 97%, 98%, 98.7% or 99% identity with a 16S rRNA gene sequence of Streptococcus salivarius.

In certain embodiments, the bacterium for determining whether the subject has an increased likelihood of a CAR T cell associated toxicity or a decreased likelihood of a CAR T cell associated toxicity is Oscillibacter ruminantium. In certain non-limiting embodiments, the bacterium can comprise one or more strains of the bacterial species Oscillibacter ruminantium, for example, found within the taxonomic group NCBI:txid1263547, or a bacterium comprising a 16S rRNA gene sequence that has at least about 94.5%, 95%, 97%, 98%, 98.7% or 99% sequence identity with a 16S rRNA gene sequence of Oscillibacter ruminantium.

In certain embodiments, the bacterium for determining whether the subject has an increased likelihood of a CAR T cell associated toxicity or a decreased likelihood of a CAR T cell associated toxicity is Eubacterium ramulus. In certain non-limiting embodiments, the bacterium can comprise one or more strains of the bacterial species Eubacterium ramulus, for example, found within the taxonomic group NCBI:txid39490, or a bacterium comprising a 16S rRNA gene sequence that has at least about 94.5%, 95%, 97%, 98%, 98.7% or 99% sequence identity with a 16S rRNA gene sequence of Eubacterium ramulus.

In certain embodiments, the bacterium for determining whether the subject has an increased likelihood of a CAR T cell associated toxicity or a decreased likelihood of a CAR T cell associated toxicity is Streptococcus gordonii. In certain non-limiting embodiments, the bacterium can comprise one or more strains of the bacterial species Streptococcus gordonii, for example, found within the taxonomic group NCBI:txid1302, or a bacterium comprising a 16S rRNA gene sequence that has at least about 94.5%, 95%, 97%, 98%, 98.7% or 99% sequence identity with a 16S rRNA gene sequence of Streptococcus gordonii.

In certain embodiments, the bacterium for determining whether the subject has an increased likelihood of a CAR T cell associated toxicity or a decreased likelihood of a CAR T cell associated toxicity is Caproiciproducens galactitolivorans. In certain non-limiting embodiments, the bacterium can comprise one or more strains of the bacterial species Caproiciproducens galactitolivorans, for example, found within the taxonomic group NCBI:txid642589, or a bacterium comprising a 16S rRNA gene sequence that has at least about 94.5%, 95%, 97%, 98%, 98.7% or 99% sequence identity with a 16S rRNA gene sequence of Caproiciproducens galactitolivorans.

In certain embodiments, the bacterium for determining whether the subject has an increased likelihood of a CAR T cell associated toxicity or a decreased likelihood of a CAR T cell associated toxicity is Clostridium hylemonae. In certain non-limiting embodiments, the bacterium can comprise one or more strains of the bacterial species Clostridium hylemonae, for example, found within the taxonomic group NCBI:txid89153, or a bacterium comprising a 16S rRNA gene sequence that has at least about 94.5%, 95%, 97%, 98%, 98.7% or 99% sequence identity with a 16S rRNA gene sequence of Clostridium hylemonae.

In certain embodiments, the bacterium for determining whether the subject has an increased likelihood of a CAR T cell associated toxicity or a decreased likelihood of a CAR T cell associated toxicity is Streptococcus oralis. In certain non-limiting embodiments, the bacterium can comprise one or more strains of the bacterial species Streptococcus oralis, for example, found within the taxonomic group NCBI:txid1303, or a bacterium comprising a 16S rRNA gene sequence that has at least about 94.5%, 95%, 97%, 98%, 98.7% or 99% sequence identity with a 16S rRNA gene sequence of Streptococcus oralis.

In certain embodiments, the bacterium for determining whether the subject has an increased likelihood of a CAR T cell associated toxicity or a decreased likelihood of a CAR T cell associated toxicity is Lutispora thermophila. In certain non-limiting embodiments, the bacterium can comprise one or more strains of the bacterial species Lutispora thermophila, for example, found within the taxonomic group NCBI:txid288966, or a bacterium comprising a 16S rRNA gene sequence that has at least about 94.5%, 95%, 97%, 98%, 98.7% or 99% sequence identity with a 16S rRNA gene sequence of Lutispora thermophila.

In certain embodiments, the bacterium for determining whether the subject has an increased likelihood of a CAR T cell associated toxicity or a decreased likelihood of a CAR T cell associated toxicity is Agathobaculum butyriciproducens. In certain non-limiting embodiments, the bacterium can comprise one or more strains of the bacterial species Agathobaculum butyriciproducens, for example, found within the taxonomic group NCBI:txid1628085, or a bacterium comprising a 16S rRNA gene sequence that has at least about 94.5%, 95%, 97%, 98%, 98.7% or 99% sequence identity with a 16S rRNA gene sequence of Agathobaculum butyriciproducens.

In certain embodiments, the bacterium for determining whether the subject has an increased likelihood of a CAR T cell associated toxicity or a decreased likelihood of a CAR T cell associated toxicity is Schaalia odontolytica. In certain non-limiting embodiments, the bacterium can comprise one or more strains of the bacterial species Schaalia odontolytica, for example, found within the taxonomic group NCBI:txid1660, or a bacterium comprising a 16S rRNA gene sequence that has at least about 94.5%, 95%, 97%, 98%, 98.7% or 99% sequence identity with a 16S rRNA gene sequence of Schaalia odontolytica.

In certain embodiments, the bacterium for determining whether the subject has an increased likelihood of a CAR T cell associated toxicity or a decreased likelihood of a CAR T cell associated toxicity is Lactobacillus salivarius. In certain non-limiting embodiments, the bacterium can comprise one or more strains of the bacterial species Lactobacillus salivarius, for example, found within the taxonomic group NCBI:txid1624, or a bacterium comprising a 16S rRNA gene sequence that has at least about 94.5%, 95%, 97%, 98%, 98.7% or 99% sequence identity with a 16S rRNA gene sequence of Lactobacillus salivarius.

In certain embodiments, a subject is identified as having an increased likelihood of a CAR T cell associated toxicity if the level of a bacterium identified as being associated with an increased likelihood of toxicity in FIG. 5, e.g., FIGS. 5G and 5O, is higher in the subject's sample than the reference level of the bacterium or spores thereof, e.g., Streptococcus salivarius, Oscillibacter ruminantium, Eubacterium ramulus, Streptococcus gordonii, Caproiciproducens galactitolivorans and/or Clostridium hylemonae. For example, but not by way of limitation, a subject is identified as having an increased likelihood of a CAR T cell associated toxicity if the level of Streptococcus salivarius and/or Oscillibacter ruminantium is higher in the subject's sample than the reference bacterium level. In certain embodiments, the reference bacterium level is the level of the bacterium or spores thereof from a subject that has been treated with a CAR T cell therapy and exhibited CAR T cell associated toxicity.

In certain embodiments, a subject is identified as having a decreased likelihood of a CAR T cell associated toxicity if the level of a bacterium identified as being associated with a decreased likelihood of toxicity in FIG. 5, e.g., FIGS. 5G and 5O, is higher in the subject's sample than the reference level of the bacterium or spores thereof, e.g., Streptococcus oralis, Lutispora thermophila, Agathobaculum butyriciproducens, Lactobacillus salivarius and/or Schaalia odontolytica. For example, but not by way of limitation, a subject is identified as having a decreased likelihood of a CAR T cell associated toxicity if the level of Lactobacillus salivarius is higher in the subject's sample than the reference level of the bacterium or spores thereof.

In certain embodiments, a subject is identified as having a decreased likelihood of a CAR T cell associated toxicity if the level of a bacterium identified as being associated with a decreased likelihood of toxicity in FIG. 5, e.g., FIGS. 5G and 5O, is higher in the subject's sample than the reference level of the bacterium or spores thereof, e.g., Streptococcus oralis, Lutispora thermophila, Agathobaculum butyriciproducens, Lactobacillus salivarius, Schaalia odontolytica, Faecalibacterium prausnitzii and/or Blautia luti. For example, but not by way of limitation, a subject is identified as having a decreased likelihood of a CAR T cell associated toxicity if the level of Lactobacillus salivarius is higher in the subject's sample than the reference level of the bacterium or spores thereof. In certain embodiments, a subject is identified as having a decreased likelihood of a CAR T cell associated toxicity if the level of Faecalibacterium prausnitzii is higher in the subject's sample than the reference level of the bacterium or spores thereof. In certain embodiments, the reference bacterium level is the level of the bacterium or spores thereof from a subject that has been treated with a CAR T cell therapy and did not exhibit CAR T cell associated toxicity.

In certain embodiments, a bacterial gene to be analyzed in a sample of the subject for determining whether the subject has an increased likelihood of a CAR T cell therapy toxicity or a decreased likelihood of a CAR T cell associated toxicity is provided in FIG. 5, e.g., FIG. 5M. For example, but not by way of limitation, the bacterial gene can be a bacterial gene involved in pentose phosphate pathway non-oxidative branch (e.g., nonoxipent PWY), L-isoleucine biosynthesis (e.g., PWY 5104), pyruvate fermentation to acetate and lactate II (PWY 5100), superpathway of glycerol degradation to 1,3-propanediol (e.g., GOLPDLCAT PWY), formaldehyde assimilation III, dihydroxyacetone cycle (e.g., PWY P185), pyrimidine deoxyribonucleotides de novo biosynthesis IV (PWY 7198), acetyl-CoA fermentation to butanoate II (e.g., PWY 5676), glycerol degradation to butanol (e.g., PWY 7003), superpathway of (R,R)-butanediol biosynthesis (e.g., PWY P125), Biotin biosynthesis II (e.g., PWY 5005), CMP-legionaminate biosynthesis (e.g., PWY 6749), chondroitin sulfate degradation I, bacterial (e.g., PWY 6572), superpathway of polyamine biosynthesis II (e.g., Polyaminsyn 3 PWY), TCA cycle, prokaryotic (e.g., TCA), pyrimidine deoxyribonucleotides de novo biosynthesis (e.g., PWY 7184), GDP-mannose biosynthesis (e.g., PWY 5659), NAD salvage pathway II (e.g., NAD biosynthesis II), CMP 3-deoxy-D-manno-octulosonate biosynthesis (e.g., PWY 1269), superpathway of pyrimidine nucleobases salvage (e.g., PWY 7208), superpathway of thiamine diphosphate biosynthesis II (e.g., PWY 6895), polyisoprenoid biosynthesis, E. coli (e.g., polyisoprensyn PWY), thiamine diphosphate salvage II (e.g., PWY 6897), superpathway of phospholipid biosynthesis I, bacteria (e.g., Phoslipsyn PWY), L-arginine biosynthesis III, via N-acetyl-L-citrulline (e.g., PWY 5154), superpathway of purine nucleotides de novo synthesis (e.g., PWY 841), superpathway of L-aspartate and L-asparagine biosynthesis (e.g., ASPASN PWY), flavin biosynthesis III, fungi (e.g., PWY 6168), Pre QO biosynthesis (e.g., PWY 6703), L-histidine degradation III (e.g., PWY 6168), superpathway of thiamine diphosphate biosynthesis III, eukaryotes (e.g., THISYNARA PWY), L-ornithine biosynthesis (e.g., Arginine Syn4 PWY), 6-hydroxymethyl-dihydropterin diphosphate biosynthesis I (e.g., PWY 6147), 6-hydroxymethyl-dihydropterin diphosphate biosynthesis III, chlamydia (e.g., PWY 7539) and/or dTDP-β-L-rhamnose biosynthesis (e.g., DTDPRHAMSYN PWY).

In certain embodiments, a subject is identified as having an increased likelihood of a CAR T cell therapy toxicity if the level of one or more genes identified as being associated with an increased likelihood of toxicity in FIG. 5M, e.g., a bacterial gene involved in pentose phosphate pathway non-oxidative branch (e.g., nonoxipent PWY), L-isoleucine biosynthesis (e.g., PWY 5104), pyruvate fermentation to acetate and lactate II (PWY 5100), superpathway of glycerol degradation to 1,3-propanediol (e.g., GOLPDLCAT PWY), formaldehyde assimilation III, dihydroxyacetone cycle (e.g., PWY P185), pyrimidine deoxyribonucleotides de novo biosynthesis IV (PWY 7198), acetyl-CoA fermentation to butanoate II (e.g., PWY 5676), glycerol degradation to butanol (e.g., PWY 7003) and/or superpathway of (R,R)-butanediol biosynthesis (e.g., PWY P125), is higher in the subject's sample than the reference level of the bacterial genes. In certain embodiments, a subject is identified as having an increased likelihood of a CAR T cell therapy toxicity if the level of one or more genes involved in pentose phosphate pathway non-oxidative branch (e.g., nonoxipent PWY), is higher in the subject's sample than the reference level of the bacterial genes.

In certain embodiments, a subject is identified as having a decreased likelihood of a CAR T cell therapy toxicity if the level of one or more genes identified as being associated with a decreased likelihood of toxicity in FIG. 5M, e.g., a bacterial gene involved in Biotin biosynthesis II (e.g., PWY 5005), CMP-legionaminate biosynthesis (e.g., PWY 6749), chondroitin sulfate degradation I, bacterial (e.g., PWY 6572), superpathway of polyamine biosynthesis II (e.g., Polyaminsyn 3 PWY), TCA cycle, prokaryotic (e.g., TCA), pyrimidine deoxyribonucleotides de novo biosynthesis (e.g., PWY 7184), GDP-mannose biosynthesis (e.g., PWY 5659), NAD salvage pathway II (e.g., NAD biosynthesis II), CMP 3-deoxy-D-manno-octulosonate biosynthesis (e.g., PWY 1269), superpathway of pyrimidine nucleobases salvage (e.g., PWY 7208), superpathway of thiamine diphosphate biosynthesis II (e.g., PWY 6895), polyisoprenoid biosynthesis, E. coli (e.g., polyisoprensyn PWY), thiamine diphosphate salvage II (e.g., PWY 6897), superpathway of phospholipid biosynthesis I, bacteria (e.g., Phoslipsyn PWY), L-arginine biosynthesis III, via N-acetyl-L-citrulline (e.g., PWY 5154), superpathway of purine nucleotides de novo synthesis (e.g., PWY 841), superpathway of L-aspartate and L-asparagine biosynthesis (e.g., ASPASN PWY), flavin biosynthesis III, fungi (e.g., PWY 6168), Pre QO biosynthesis (e.g., PWY 6703), L-histidine degradation III (e.g., PWY 6168), superpathway of thiamine diphosphate biosynthesis III, eukaryotes (e.g., THISYNARA PWY), L-ornithine biosynthesis (e.g., Arginine Syn4 PWY), 6-hydroxymethyl-dihydropterin diphosphate biosynthesis I (e.g., PWY 6147), 6-hydroxymethyl-dihydropterin diphosphate biosynthesis III, chlamydia (e.g., PWY 7539) and/or dTDP-β-L-rhamnose biosynthesis (e.g., DTDPRHAMSYN PWY), is higher in the subject's sample than the reference level of the bacterial genes. In certain embodiments, the reference level of the bacterial gene is the level of bacterial gene from a subject that has been treated with a CAR T cell therapy and did not exhibit CAR T cell therapy toxicity.

In certain embodiments, a method of identifying a subject as having an increased likelihood of a CAR T cell associated toxicity or a decreased likelihood of a CAR T cell associated toxicity includes determining whether the subject has been administered an antibiotic prior to a CAR T cell therapy, e.g., initiation of a CAR T cell therapy. In certain embodiments, the antibiotic is administered less than about 8 weeks, less than about 7 weeks, less than about 6 weeks, less than about 5 weeks, less than about 4 weeks, less than about 3 weeks, less than about 2 weeks, less than about 1 week prior to a CAR T cell therapy, e.g., initiation of a CAR T cell therapy. In certain embodiments, the antibiotic is administered less than about 4 weeks prior to a CAR T cell therapy, e.g., initiation of a CAR T cell therapy. In certain embodiments, the antibiotic is administered about 4 weeks prior to CAR T cell therapy, e.g., initiation of a CAR T cell therapy. In certain embodiments, a subject is identified as having an increased likelihood of a CAR T cell associated toxicity if the subject has been administered piperacillin-tazobactam, imipenem-cilastatin, meropenem or a combination thereof prior to a CAR T cell therapy. In certain embodiments, a subject having NHL is identified as having an increased likelihood of a CAR T cell associated toxicity if the subject has been administered piperacillin-tazobactam, imipenem-cilastatin, meropenem or a combination thereof prior to a CAR T cell therapy.

In certain embodiments, a subject that is identified as having an increased likelihood of CAR T cell associated toxicity can be monitored for signs of severe toxicity, can receive prophylactic treatments to decrease the chances or effects of severe toxicity, e.g., by administration of a therapeutic bacteria disclosed herein, without unduly hampering the effectiveness of CAR T cell therapy, or a combination thereof.

An increased or decreased level of a bacterium or spores thereof or of a bacterial gene described herein is determined with respect to a reference bacterium or spores thereof level or a reference bacterial gene level. In certain embodiments, the level (e.g., the measured level and the reference level) can be based on a relative abundance in the intestinal microbiome. For example, but not by way of limitation, the level can represent a percentage of the bacterium or spores thereof of all the bacteria or spores thereof in the intestinal microbiome. In certain embodiments, the level can also be an absolute number.

In certain embodiments, the reference level is the level of a bacterium or spores thereof or of a bacterial gene from (a) a population of subjects that are candidates for a CAR T cell therapy, (b) subjects that have been treated with a CAR T cell therapy and exhibited cancer survival, (c) subjects that have been treated with a CAR T cell therapy and did not exhibit cancer survival, (d) subjects that have been treated with a CAR T cell therapy and exhibited CAR T cell associated toxicity and/or (e) subjects that have been treated with a CAR T cell therapy and did not exhibit CAR T cell associated toxicity. In certain embodiments, the reference level is the level of a bacterium or spores thereof or of a bacterial gene from a sample of the same subject collected at an earlier time point. In certain embodiments, the reference level can be based on a prior test in the same subject, or on levels found in a patient population, such as subjects who are candidates for CAR T cell therapy or subject with cancer that have been treated with a CAR T cell therapy and exhibited cancer survival. In certain embodiments, a reference level can be the abundance of a bacterium or spores thereof or of a bacterial gene in the microbiota of a subject with cancer who has survived for at least about 100 days following a CAR T cell therapy.

In certain embodiments, the methods can further include treating the subject that is identified as having an increased likelihood of cancer survival following CAR T cell therapy or a decreased likelihood of a CAR T cell associated toxicity with a CAR T cell therapy. Any CAR T cell therapy known in the art can be used with the presently disclosed subject matter. In certain embodiments, the CAR T cell therapy can include a CAR T cell comprising an extracellular binding domain that binds to mucin 16 (MUC16), B-cell maturation antigen (BCMA), CD19 or a combination thereof. In certain embodiments, the CAR T cell therapy is a CD19 CAR T cell therapy. In certain embodiments, the CAR T cell therapy can comprise a CD28 or 4-1BB co-stimulatory domain.

In certain embodiments, the methods can further include treating the subject identified as having a decreased likelihood of cancer survival following CAR T cell therapy or an increased likelihood of a CAR T cell associated toxicity with a therapeutic bacteria disclosed herein or a pharmaceutical composition thereof, e.g., the compositions disclosed in Section IV. In certain embodiments, the subject can receive prophylactically therapeutic bacteria or a pharmaceutical composition thereof as described herein prior to administration of a CAR T cell therapy, prior to harvesting of cells for modification, between harvesting cells and administration of genetically modified T cells or after administration of genetically modified T cells in CAR T cell therapy.

In certain embodiments, the subject has a cancer. In certain embodiments, the cancer is selected from the group consisting of acute lymphoblastic leukemia, acute myelogenous leukemia, biliary cancer, breast cancer, cervical cancer, chronic lymphocytic leukemia, chronic myelogenous leukemia, colorectal cancer, endometrial cancer, esophageal, gastric, head and neck cancer, Hodgkin's lymphoma, lung cancer, medullary thyroid cancer, non-Hodgkin's lymphoma, multiple myeloma, renal cancer, ovarian cancer, pancreatic cancer, glioma, melanoma, liver cancer, prostate cancer, and urinary bladder cancer, CD19 malignancies, and other B cell-related or hematologic malignancies. In certain embodiments, the cancer is an ovarian cancer, a multiple myeloma or a B-cell malignancy and any combinations thereof. Non-limiting examples of B-cell malignancies include acute lymphoblastic leukemia (ALL), chronic lymphoblastic leukemia (CLL) or non-Hodgkin lymphoma (NHL)). In certain embodiments, the cancer is ALL. In certain embodiments, the cancer is NHL.

In certain embodiments, the level or the abundance of a bacterium can be determined by quantification of bacterial nucleic acid molecules (e.g., DNA or RNA molecules) in the sample. In certain embodiments, the bacterial DNA or RNA comprises 16S rRNA or RNA encoded by a bacterial gene unique to the bacterial species. In certain embodiments, the bacterial DNA (e.g., 16S rDNA) or RNA level (e.g., 16S rRNA) is determined by a sequencing method, e.g., metagenomic sequencing or shotgun metagenomic sequencing. In certain embodiments, the sequencing is performed using an Illumina MiSeq platform or Illumina HiSeq 2000 platform. In certain embodiments, the bacterial DNA or RNA level (e.g., copy number) is determined by an amplification-based method, e.g., by polymerase chain reaction (PCR), including reverse transcription-polymerase chain reaction (RT-PCR) for RNA quantitative analysis. In certain embodiments, amplification of the bacterial DNA or RNA in a sample can be accomplished by any known method, including but not limited to ligase chain reaction (LCR), transcription-mediated amplification, and self-sustained sequence replication or nucleic acid sequence-based amplification (NASBA). In certain embodiments, the level of a bacterial DNA or RNA level can be determined by size fractionation (e.g., gel electrophoresis), whether or not proceeded by an amplification step. In certain embodiments, the level of a bacterial nucleic acid molecule (e.g., DNA or RNA molecules) level can be determined by sequence-specific probe hybridization. In certain embodiments, the level of a bacterial DNA or RNA level can be determined by mass spectroscopy, PCR, microarray hybridization, thermal sequencing, capillary array sequencing or solid phase sequencing.

In certain embodiments, the level or the abundance of the bacterium refers to a relative abundance of the bacterium in a sample. The relative abundance of a bacterium refers to the proportion occupied by the particular bacterium in the whole bacterial flora in the sample. The relative abundance of a bacterium can be determined from, for example, the total number of bacterial cells constituting the bacterial flora and the number of the particular bacterial cells included in the bacterial flora. More specifically, for example, genes having a nucleotide sequence that is common in the bacteria included in the bacterial flora and nucleotide sequences characteristic to each bacterial species (for example, 16S rRNA gene) are comprehensively decoded, and the relative abundance of a particular bacterium can be determined by designating the total number of decoded genes and the total number of genes belonging to particular bacterial species as the total number of bacterial cells constituting the bacterial flora and the number of particular bacterial cells, respectively.

In certain embodiments, the level of a bacterial gene is determined by measuring a level of a bacterial nucleic acids include DNA and RNA including at least a portion of the bacterial gene, a bacterial mRNA or cDNA that is transcribed from the bacterial gene, or a sequence complementary or homologous thereto (including but not limited to antisense or small interfering RNA). Said nucleic acid can include natural nucleotides and can optionally include nucleotide bases which are not naturally occurring. In certain embodiments, the level of a bacterial gene is determined by measuring a level of a bacterial protein that is encoded by the bacterial gene.

Any suitable methods known in the art for measuring nucleic acid and protein levels can be used with the presently disclosed methods. In certain embodiments, methods for measuring nucleic acid levels include, but not limited to, real-time PCR (RT-PCR), quantitative PCR, quantitative real-time polymerase chain reaction (qRT-PCR), fluorescent PCR, RT-MSP (RT methylation specific polymerase chain reaction), PicoGreen™ (Molecular Probes, Eugene, OR) detection of DNA, radioimmunoassay or direct radio-labeling of DNA, in situ hybridization visualization, fluorescent in situ hybridization (FISH), microarray, sequencing. In certain embodiments, methods for measuring protein levels include, but are not limited to, mass spectrometry techniques, 1-D or 2-D gel-based analysis systems, chromatography, enzyme linked immunosorbent assays (ELISAs), radioimmunoassays (RIA), enzyme immunoassays (EIA), Western Blotting, immunoprecipitation and immunohistochemistry.

In certain embodiments, the amount and/or type of bacteria present in a sample can be determined by measuring the amount or presence of bacterial nucleic acid specific for the type of bacteria, such as 16S rRNA. In certain embodiments, the amount and/or type of bacteria present in a sample can be determined by shotgun sequencing of bacterial DNA, PCR amplification of specific genes carried by the bacteria, quantitative PCR of transcripts expressed specifically by the bacteria, antibody based methods of bacterial detection, metabolomic detection of bacterial metabolites, proteomic detection of bacterial proteins, and/or by methods of culturing the microbiota sample.

In certain embodiments, the amount and/or type of bacterial genes present in a sample can be determined by PCR amplification of the specific genes or quantitative PCR of transcripts expressed specifically by the bacteria, or by tests for the effects of the expression of such genes, such as degradation of secondary bile acids by the microbiota sample.

In certain embodiments, the subject is a candidate for a CAR T cell therapy and has not received the CAR T cell therapy. In certain embodiments, the subject has previously received a CAR T cell therapy. In certain embodiments, the subject is receiving a CAR T cell therapy.

In certain embodiments, the microbiota sample can be collected from the subject up to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more days before treatment with a CAR T cell therapy. In certain embodiments, the microbiota sample can be collected from the subject up to about 30 days before treatment with a CAR T cell therapy. For example, but not by way of limitation, the microbiota sample can be collected from the subject up to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more days before cells will be harvested from the subject for modification into CAR T cell therapy, or before modified T cells will be administered to the patient in CAR T cell therapy. The microbiota sample can be collected from the subject after cells are harvested from the patient for CAR T cell therapy, but prior to administration of the modified T cell. The microbiota sample can also be collected from the patient 1, 2, 3, 4, 5, 6, 7, 8, 9, 10, 11, 12, 13 or 14 days after administration of the modified T cells to the patient, or after the patient exhibits a symptom of toxicity.

In certain embodiments, the sample, e.g., microbiota sample, from the subject can be a fecal sample or an intestinal content sample, for example, a rectal swab.

In certain embodiments, the subject is a human subject.

### III. Therapeutic Bacteria

The present disclosure provides therapeutic bacteria or spores thereof for administration to a subject. In certain embodiments, the present disclosure provides therapeutic bacteria or spores thereof for treating a cancer in combination with a CAR T cell therapy. In certain embodiments, the therapeutic bacteria can be used for improving a subject's likelihood of cancer survival following CAR T cell therapy and/or reducing a subject's risk of toxicity after CAR T cell therapy.

In certain embodiments, the therapeutic bacteria can include one or more bacteria associated with an increased likelihood of cancer survival, e.g., as disclosed in FIG. 5. In certain embodiments, the therapeutic bacteria can include one or more bacteria comprising a gene associated with an increased likelihood of cancer survival, e.g., as disclosed in FIG. 5.

In certain embodiments, the therapeutic bacteria can include one or more bacteria associated with a decreased likelihood of a CAR T cell therapy toxicity, e.g., as disclosed in FIG. 5. In certain embodiments, the therapeutic bacteria of the present disclosure can include one or more bacteria comprising a gene associated with a decreased likelihood of a CAR T cell therapy toxicity, e.g., as disclosed in FIG. 5.

In certain embodiments, the therapeutic bacteria for use in the present disclosure include one or more bacterium or spores thereof of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Streptococcus oralis, Lutispora thermophila, Agathobaculum butyriciproducens, Lactobacillus salivarius, Schaalia odontolytica, Ruminococcus bromii, Longicatena caecimuris, Bifidobacterium breve, Blautia luti and/or Faecalibacterium prausnitzii. In certain embodiments, the therapeutic bacteria include one or more bacterium or spores thereof comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Streptococcus oralis, Lutispora thermophila, Agathobaculum butyriciproducens, Lactobacillus salivarius, Ruminococcus bromii, Longicatena caecimuris, Bifidobacterium breve, Blautia luti and/or Faecalibacterium prausnitzii.

In certain embodiments, the therapeutic bacteria for improving a subject's likelihood of cancer survival include one or more bacterium or spores thereof of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti, Faecalibacterium prausnitzii or a combination thereof or bacteria comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti and/or Faecalibacterium prausnitzii.

In certain embodiments, the therapeutic bacteria for reducing a subject's risk of toxicity associated with CAR T cell therapy include one or more bacterium or spores thereof of Streptococcus oralis, Lutispora thermophila, Agathobaculum butyriciproducens, Lactobacillus salivarius, Schaalia odontolytica, Blautia luti, Faecalibacterium prausnitzii or a combination thereof or bacteria comprising a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica, Blautia luti and/or Faecalibacterium prausnitzii.

In certain embodiments, the therapeutic bacteria for reducing a subject's risk of toxicity associated with CAR T cell therapy include one or more bacterium or spores thereof of Lutispora thermophila, Lactobacillus salivarius, Schaalia odontolytica, Blautia luti, Faecalibacterium prausnitzii or a combination thereof or bacteria comprising a 16S rRNA gene sequence having at least 94.5%, 95%, 97%, 98%, 98.7% or 99% sequence identity to the 16S rRNA gene sequence of Lutispora thermophila, Lactobacillus salivarius, Schaalia odontolytica, Blautia luti and/or Faecalibacterium prausnitzii.

In certain embodiments, the therapeutic bacteria comprises Ruminococcus gnavus, Streptococcus salivarius, Oscillibacter ruminantium, Ruminococcus bromii, Blautia luti, Faecalibacterium prausnitzii or a combination thereof or bacteria comprising a 16S rRNA gene sequence having at least 94.5%, 95%, 97%, 98%, 98.7% or 99% sequence identity to the 16S rRNA gene sequence of Ruminococcus gnavus, Streptococcus salivarius, Oscillibacter ruminantium, Ruminococcus bromii, Blautia luti and/or Faecalibacterium prausnitzii.

In certain embodiments, a therapeutic bacteria can include bacteria comprising one or more genes involved in purine nucleosides degradation (e.g., PWY 1296), chorismate biosynthesis (e.g., ARO PWY), antigen building blocks biosynthesis (e.g., O Antigen Pathway), Calvin Benson Bassham cycle (e.g., Calvin PWY), peptidoglycan biosynthesis IV (e.g., PWY 6471), adenine and adenosine salvage III (e.g., PWY 6609), superpathway of aromatic amino acid biosynthesis (e.g., COMPLETE ARO PWY), UDP N-acetyl D glucosamine biosynthesis (e.g., UDPNAGSYN PWY), chorismate biosynthesis from 3-dehydroguinate (e.g., PWY 6163), L-methionine biosynthesis III (e.g., HSERMETANA PWY), peptidoglycan biosynthesis V beta-lactam resistance (e.g., PWY 6470) and/or superpathway of pyrimidine ribonucleosides salvage (e.g., PWY 7196). In certain embodiments, a therapeutic bacteria can include bacteria comprising one or more genes involved in aromatic amino acid biosynthesis (e.g., chorismate biosynthesis and the superpathway of aromatic amino acid biosynthesis (COMPLETE ARO PWY)), peptidoglycan biosynthesis (peptidoglycan biosynthesis IV (PWY 6471) and/or peptidoglycan biosynthesis V beta-lactam resistance (PWY 6470)) (e.g., peptidoglycan biosynthesis IV (PWY 6471)). In certain embodiments, a therapeutic bacteria can include bacteria comprising a gene involved in peptidoglycan biosynthesis IV (e.g., PWY 6471).

In certain embodiments, a therapeutic bacteria can include bacteria comprising one or more genes involved in Biotin biosynthesis II (e.g., PWY 5005), CMP-legionaminate biosynthesis (e.g., PWY 6749), chondroitin sulfate degradation I, bacterial (e.g., PWY 6572), superpathway of polyamine biosynthesis II (e.g., Polyaminsyn 3 PWY), TCA cycle, prokaryotic (e.g., TCA), pyrimidine deoxyribonucleotides de novo biosynthesis (e.g., PWY 7184), GDP-mannose biosynthesis (e.g., PWY 5659), NAD salvage pathway II (e.g., NAD biosynthesis II), CMP 3-deoxy-D-manno-octulosonate biosynthesis (e.g., PWY 1269), superpathway of pyrimidine nucleobases salvage (e.g., PWY 7208), superpathway of thiamine diphosphate biosynthesis II (e.g., PWY 6895), polyisoprenoid biosynthesis, E. coli (e.g., polyisoprensyn PWY), thiamine diphosphate salvage II (e.g., PWY 6897), superpathway of phospholipid biosynthesis I, bacteria (e.g., Phoslipsyn PWY), L-arginine biosynthesis III, via N-acetyl-L-citrulline (e.g., PWY 5154), superpathway of purine nucleotides de novo synthesis (e.g., PWY 841), superpathway of L-aspartate and L-asparagine biosynthesis (e.g., ASPASN PWY), flavin biosynthesis III, fungi (e.g., PWY 6168), Pre QO biosynthesis (e.g., PWY 6703), L-histidine degradation III (e.g., PWY 6168), superpathway of thiamine diphosphate biosynthesis III, eukaryotes (e.g., THISYNARA PWY), L-ornithine biosynthesis (e.g., Arginine Syn4 PWY), 6-hydroxymethyl-dihydropterin diphosphate biosynthesis I (e.g., PWY 6147), 6-hydroxymethyl-dihydropterin diphosphate biosynthesis III, chlamydia (e.g., PWY 7539) and/or dTDP-β-L-rhamnose biosynthesis (e.g., DTDPRHAMSYN PWY).

In certain embodiments, at least one, at least two, at least three, at least four, at least five, at least six, at least seven, at least eight, at least nine, at least ten, at least eleven or at least twelve bacteria species can be administered to a subject, e.g., for improving a subject's likelihood of cancer survival. For example, but not way of limitation, Ruminococcus gnavus can be administered to a subject. In certain embodiments, Streptococcus salivarius can be administered to a subject. In certain embodiments, Oscillibacter ruminantium can be administered to a subject. In certain embodiments, Blautia luti can be administered to a subject. In certain embodiments, Faecalibacterium prausnitzii can be administered to a subject. In certain embodiments, Ruminococcus bromii can be administered to a subject. In certain embodiments, Ruminococcus gnavus and Streptococcus salivarius can be administered to a subject. In certain embodiments, Ruminococcus gnavus and Oscillibacter ruminantium can be administered to a subject. In certain embodiments, Ruminococcus gnavus, Streptococcus salivarius and Oscillibacter ruminantium can be administered to a subject. In certain embodiments, Streptococcus salivarius and Oscillibacter ruminantium can be administered to a subject.

In certain embodiments, at least one, at least two, at least three, at least four, at least five, at least six, at least seven, at least eight, at least nine, at least ten, at least eleven, at least twelve or at least thirteen bacteria species can be administered to a subject, e.g., for improving a subject's likelihood of cancer survival. For example, but not way of limitation, Ruminococcus gnavus can be administered to a subject. In certain embodiments, Blautia schinkii can be administered to a subject. In certain embodiments, Bacteroides finegoldii can be administered to a subject. In certain embodiments, Clostridium glycyrrhizinilyticum can be administered to a subject. In certain embodiments, Clostridium saccharolyticum can be administered to a subject. In certain embodiments, Clostridium celerecrescens can be administered to a subject. In certain embodiments, Anaeromassilibacillus senegalensis can be administered to a subject. In certain embodiments, Clostridium methoxybenzovorans can be administered to a subject. In certain embodiments, Faecalibacterium prausnitzii can be administered to a subject. In certain embodiments, Blautia luti can be administered to a subject. In certain embodiments, Longicatena caecimuris can be administered to a subject. In certain embodiments, Bifidobacterium breve can be administered to a subject. In certain embodiments, Ruminococcus bromii can be administered to a subject.

In certain embodiments, at least one, at least two, at least three, at least four, at least five, at least six, at least seven, at least eight, at least nine, at least ten, at least eleven or at least twelve bacteria species can be administered to a subject, e.g., for reducing a subject's risk of toxicity associated with CAR T cell therapy. For example, but not way of limitation, Streptococcus oralis can be administered to a subject. In certain embodiments, Lutispora thermophila can be administered to a subject. In certain embodiments, Agathobaculum butyriciproducens can be administered to a subject. In certain embodiments, Lactobacillus salivarius can be administered to a subject. In certain embodiments, Schaalia odontolytica can be administered to a subject. In certain embodiments, Blautia luti can be administered to a subject. In certain embodiments, Faecalibacterium prausnitzii can be administered to a subject. In certain embodiments, Lactobacillus salivarius and Lutispora thermophila can be administered to a subject. In certain embodiments, Lactobacillus salivarius, Schaalia odontolytica and Lutispora thermophila can be administered to a subject.

In certain embodiments, the present disclosure provides a composition comprising at least one of the presently disclosed bacteria or spores thereof, or a cluster including at least one of the presently disclosed bacteria.

In certain embodiments, the presently disclosed therapeutic bacteria can be administered in the vegetative or dormant state, or as spores, or a mixture thereof.

Therapeutic bacteria as described herein, any combinations thereof, or a cluster including any one or more of the therapeutic bacteria, can be administered in the form of purified bacteria or spores or other progenitors thereof, or alternatively can be administered as a constituent in a mixture of types of bacteria, optionally including one or more species or cluster of additional bacteria, for example, probiotic bacteria, a probiotic yeast, prebiotic, postbiotic and/or antibiotic.

The presently disclosed therapeutic bacteria can be administered in the form of a liquid, a suspension, a dried (e.g., lyophilized) powder, a tablet, a capsule, or a suppository, and can be administered orally, nasogastrically, or rectally.

In certain embodiments, the presently disclosed therapeutic bacteria can be administered in a food product, for example, a yogurt food product. A “food product” can mean a product or composition that is intended for consumption by a human or a non-human animal. Such food products include any food, feed, snack, food supplement, liquid, beverage, treat, toy (chewable and/or consumable toys), meal substitute or meal replacement.

The present disclosure further provides a composition including an isolated and/or purified therapeutic bacteria, a combination of any isolated therapeutic bacteria with one another or a cluster including any one or more of the isolated therapeutic bacteria. In certain embodiments, the bacteria can be in a formulation for administration to a patient.

The present disclosure provides a composition including an isolated therapeutic bacteria, which can be one or more of the therapeutic bacteria described herein, but alternate or additional bacteria can be included in other compositions described herein, for example, bacteria which can be naturally occurring bacteria that are in a cluster with any one or more of therapeutic bacteria.

### IV. Pharmaceutical Compositions

The present disclosure provides for pharmaceutical compositions including one or more therapeutic bacteria disclosed herein. In certain embodiments, a pharmaceutical composition of the present disclosure includes a therapeutic bacteria or spore thereof and a pharmaceutical carrier.

In certain embodiments, a pharmaceutical composition of the present disclosure can include one or more bacteria associated with an increased likelihood of cancer survival, e.g., as disclosed in FIG. 5. In certain embodiments, a pharmaceutical composition of the present disclosure can include one or more bacteria comprising a gene associated with an increased likelihood of cancer survival, e.g., as disclosed in FIG. 5.

In certain embodiments, a pharmaceutical composition of the present disclosure can include one or more bacteria associated with a decreased likelihood of a CAR T cell therapy toxicity, e.g., as disclosed in FIG. 5. In certain embodiments, a pharmaceutical composition of the present disclosure can include one or more bacteria comprising a gene associated with a decreased likelihood of a CAR T cell therapy toxicity, e.g., as disclosed in FIG. 5.

In certain embodiments, a pharmaceutical composition of the present disclosure can include one or more therapeutic bacteria disclosed in Section III.

In certain embodiments, a pharmaceutical composition of the present disclosure can include one or more therapeutic bacteria or spores thereof, e.g., isolated and/or purified therapeutic bacteria or spores thereof, comprising Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Streptococcus oralis, Lutispora thermophila, Agathobaculum butyriciproducens, Lactobacillus salivarius, Schaalia odontolytica, Ruminococcus bromii, Longicatena caecimuris, Bifidobacterium breve, Blautia luti, Faecalibacterium prausnitzii or a combination thereof. In certain embodiments, a pharmaceutical composition of the present disclosure can include one or more therapeutic bacteria or spores thereof comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Streptococcus oralis, Lutispora thermophila, Agathobaculum butyriciproducens, Lactobacillus salivarius, Schaalia odontolytica, Ruminococcus bromii, Longicatena caecimuris, Bifidobacterium breve, Blautia luti and/or Faecalibacterium prausnitzii.

In certain embodiments, a pharmaceutical composition for improving a subject's likelihood of cancer survival can comprise one or more bacterium or spores thereof, e.g., isolated and/or purified therapeutic bacteria or spores thereof, of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti, Faecalibacterium prausnitzii or a combination thereof. In certain embodiments, a pharmaceutical composition for improving a subject's likelihood of cancer survival can comprise one or more bacterium or spores thereof, e.g., isolated and/or purified therapeutic bacteria or spores thereof, comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti and/or Faecalibacterium prausnitzii.

In certain embodiments, a pharmaceutical composition for reducing a subject's likelihood of toxicity after CAR T cell therapy can comprise one or more therapeutic bacterium or spores thereof, e.g., isolated and/or purified therapeutic bacteria or spores thereof, of Streptococcus oralis, Lutispora thermophila, Agathobaculum butyriciproducens, Lactobacillus salivarius, Schaalia odontolytica, Blautia luti, Faecalibacterium prausnitzii or a combination thereof. In certain embodiments, a pharmaceutical composition for reducing a subject's likelihood of toxicity after CAR T cell therapy can comprise one or more therapeutic bacterium or spores thereof, e.g., isolated and/or purified therapeutic bacteria or spores thereof, comprising a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica, Blautia luti and/or Faecalibacterium prausnitzii.

In certain embodiments, a pharmaceutical composition of the present disclosure can include a therapeutic bacteria, e.g., Ruminococcus gnavus, Streptococcus salivarius, Oscillibacter ruminantium, Ruminococcus bromii, Blautia luti, Faecalibacterium prausnitzii or a combination thereof or bacteria having a 16S rRNA gene sequence having at least 94.5%, 95%, 97%, 98%, 98.7% or 99% sequence identity to the 16S rRNA gene sequence of Ruminococcus gnavus, Streptococcus salivarius, Oscillibacter ruminantium, Ruminococcus bromii, Blautia luti and/or Faecalibacterium prausnitzii.

In certain embodiments, a pharmaceutical composition of the present disclosure can include a therapeutic bacteria, e.g., Ruminococcus gnavus, Streptococcus oralis, Lutispora thermophila, Ruminococcus bromii, Blautia luti, Faecalibacterium prausnitzii or a combination thereof or bacteria having a 16S rRNA gene sequence having at least 94.5%, 95%, 97%, 98%, 98.7% or 99% sequence identity to the 16S rRNA gene sequence of Ruminococcus gnavus, Streptococcus oralis, Lutispora thermophila, Ruminococcus bromii, Blautia luti and/or Faecalibacterium prausnitzii.

In certain embodiments, a pharmaceutical composition of the present disclosure can include a therapeutic bacteria, e.g., Ruminococcus gnavus, Lactobacillus salivarius, Lutispora thermophila, Ruminococcus bromii, Blautia luti, Faecalibacterium prausnitzii or a combination thereof or bacteria having a 16S rRNA gene sequence having at least 94.5%, 95%, 97%, 98%, 98.7% or 99% sequence identity to the 16S rRNA gene sequence of Ruminococcus gnavus, Lactobacillus salivarius, Lutispora thermophila, Ruminococcus bromii, Blautia luti and/or Faecalibacterium prausnitzii.

In certain embodiments, a pharmaceutical composition of the present disclosure can include a therapeutic bacteria, e.g., Blautia luti, Faecalibacterium prausnitzii or a combination thereof or bacteria having a 16S rRNA gene sequence having at least 94.5%, 95%, 97%, 98%, 98.7% or 99% sequence identity to the 16S rRNA gene sequence of Blautia luti and/or Faecalibacterium prausnitzii.

In certain embodiments, a pharmaceutical composition of the present disclosure can include a therapeutic bacteria, e.g., Ruminococcus bromii, Blautia luti, Faecalibacterium prausnitzii or a combination thereof or bacteria having a 16S rRNA gene sequence having at least 94.5%, 95%, 97%, 98%, 98.7% or 99% sequence identity to the 16S rRNA gene sequence of Ruminococcus bromii, Blautia luti and/or Faecalibacterium prausnitzii.

In certain embodiments, a pharmaceutical composition of the present disclosure can include a therapeutic bacteria, e.g., Ruminococcus gnavus, Streptococcus oralis, Lutispora thermophila or a combination thereof or bacteria having a 16S rRNA gene sequence having at least 94.5%, 95%, 97%, 98%, 98.7% or 99% sequence identity to the 16S rRNA gene sequence of Ruminococcus gnavus, Streptococcus oralis and/or Lutispora thermophila.

In certain embodiments, a pharmaceutical composition of the present disclosure can include Ruminococcus gnavus or a bacterium comprising a 16S rRNA gene sequence having at least 94.5%, 95%, 97%, 98%, 98.7% or 99% sequence identity to the 16S rRNA gene sequence of Ruminococcus gnavus.

In certain embodiments, a pharmaceutical composition of the present disclosure can include Ruminococcus bromii or a bacterium comprising a 16S rRNA gene sequence having at least 94.5%, 95%, 97%, 98%, 98.7% or 99% sequence identity to the 16S rRNA gene sequence of Ruminococcus bromii.

In certain embodiments, a pharmaceutical composition of the present disclosure can include Blautia luti or a bacterium comprising a 16S rRNA gene sequence having at least 94.5%, 95%, 97%, 98%, 98.7% or 99% sequence identity to the 16S rRNA gene sequence of Blautia luti.

In certain embodiments, a pharmaceutical composition of the present disclosure can include Faecalibacterium prausnitzii or a bacterium comprising a 16S rRNA gene sequence having at least 94.5%, 95%, 97%, 98%, 98.7% or 99% sequence identity to the 16S rRNA gene sequence of Faecalibacterium prausnitzii.

In certain embodiments, a pharmaceutical composition of the present disclosure can include Streptococcus salivarius or a bacterium comprising a 16S rRNA gene sequence having at least 94.5%, 95%, 97%, 98%, 98.7% or 99% sequence identity to the 16S rRNA gene sequence of Streptococcus salivarius.

In certain embodiments, a pharmaceutical composition of the present disclosure can include Oscillibacter ruminantium or a bacterium comprising a 16S rRNA gene sequence having at least 94.5%, 95%, 97%, 98%, 98.7% or 99% sequence identity to the 16S rRNA gene sequence of Oscillibacter ruminantium.

In certain embodiments, a pharmaceutical composition of the present disclosure can include Streptococcus oralis or a bacterium comprising a 16S rRNA gene sequence having at least 94.5%, 95%, 97%, 98%, 98.7% or 99% sequence identity to the 16S rRNA gene sequence of Streptococcus oralis.

In certain embodiments, a pharmaceutical composition of the present disclosure can include Lutispora thermophila or a bacterium comprising a 16S rRNA gene sequence having at least 94.5%, 95%, 97%, 98%, 98.7% or 99% sequence identity to the 16S rRNA gene sequence of Lutispora thermophila.

In certain embodiments, a pharmaceutical composition of the present disclosure can include Agathobaculum butyriciproducens or a bacterium comprising a 16S rRNA gene sequence having at least 94.5%, 95%, 97%, 98%, 98.7% or 99% sequence identity to the 16S rRNA gene sequence of Agathobaculum butyriciproducens.

In certain embodiments, a pharmaceutical composition of the present disclosure can include Lactobacillus salivarius or a bacterium comprising a 16S rRNA gene sequence having at least 94.5%, 95%, 97%, 98%, 98.7% or 99% sequence identity to the 16S rRNA gene sequence of Lactobacillus salivarius.

In certain embodiments, a pharmaceutical composition of the present disclosure can include Schaalia odontolytica or a bacterium comprising a 16S rRNA gene sequence having at least 94.5%, 95%, 97%, 98%, 98.7% or 99% sequence identity to the 16S rRNA gene sequence of Schaalia odontolytica.

In certain embodiments, a pharmaceutical composition of the present disclosure can include Ruminococcus gnavus and Streptococcus salivarius or bacteria comprising a 16S rRNA gene sequence having at least 94.5%, 95%, 97%, 98%, 98.7% or 99% sequence identity to the 16S rRNA gene sequence of Ruminococcus gnavus and/or Streptococcus salivarius. In certain embodiments, a pharmaceutical composition of the present disclosure can include Ruminococcus gnavus and Oscillibacter ruminantium or bacteria comprising a 16S rRNA gene sequence having at least 94.5%, 95%, 97%, 98%, 98.7% or 99% sequence identity to the 16S rRNA gene sequence of Ruminococcus gnavus and/or Oscillibacter ruminantium.

In certain embodiments, a pharmaceutical composition of the present disclosure can include Ruminococcus gnavus and Lactobacillus salivarius or bacteria comprising a 16S rRNA gene sequence having at least 94.5%, 95%, 97%, 98%, 98.7% or 99% sequence identity to the 16S rRNA gene sequence of Ruminococcus gnavus and/or Lactobacillus salivarius. In certain embodiments, a pharmaceutical composition of the present disclosure can include Ruminococcus gnavus and Lutispora thermophila or bacteria comprising a 16S rRNA gene sequence having at least 94.5%, 95%, 97%, 98%, 98.7% or 99% sequence identity to the 16S rRNA gene sequence of Ruminococcus gnavus and/or Lutispora thermophila.

In certain embodiments, a pharmaceutical composition of the present disclosure can include Streptococcus salivarius and Oscillibacter ruminantium or bacteria comprising a 16S rRNA gene sequence having at least 94.5%, 95%, 97%, 98%, 98.7% or 99% sequence identity to the 16S rRNA gene sequence of Streptococcus salivarius and/or Oscillibacter ruminantium.

In certain embodiments, a pharmaceutical composition of the present disclosure can include Ruminococcus gnavus, Streptococcus salivarius and Oscillibacter ruminantium or bacteria comprising a 16S rRNA gene sequence having at least 94.5%, 95%, 97%, 98%, 98.7% or 99% sequence identity to the 16S rRNA gene sequence of Ruminococcus gnavus, Streptococcus salivarius and/or Oscillibacter ruminantium.

In certain embodiments, a pharmaceutical composition of the present disclosure can include Ruminococcus gnavus, Lactobacillus salivarius and Lutispora thermophila or bacteria comprising a 16S rRNA gene sequence having at least 94.5%, 95%, 97%, 98%, 98.7% or 99% sequence identity to the 16S rRNA gene sequence of Ruminococcus gnavus, Lactobacillus salivarius and/or Lutispora thermophila.

In certain embodiments, a pharmaceutical composition of the present disclosure can include bacteria comprising one or more genes involved in purine nucleosides degradation (e.g., PWY 1296), chorismate biosynthesis (e.g., ARO PWY), antigen building blocks biosynthesis (e.g., O Antigen Pathway), Calvin Benson Bassham cycle (e.g., Calvin PWY), peptidoglycan biosynthesis IV (e.g., PWY 6471), adenine and adenosine salvage III (e.g., PWY 6609), superpathway of aromatic amino acid biosynthesis (e.g., COMPLETE ARO PWY), UDP N-acetyl D glucosamine biosynthesis (e.g., UDPNAGSYN PWY), chorismate biosynthesis from 3-dehydroguinate (e.g., PWY 6163), L-methionine biosynthesis III (e.g., HSERMETANA PWY), peptidoglycan biosynthesis V beta-lactam resistance (e.g., PWY 6470) and/or superpathway of pyrimidine ribonucleosides salvage (e.g., PWY 7196). In certain embodiments, a pharmaceutical composition of the present disclosure can include bacteria comprising one or more genes involved in aromatic amino acid biosynthesis (e.g., chorismate biosynthesis and the superpathway of aromatic amino acid biosynthesis (COMPLETE ARO PWY)), peptidoglycan biosynthesis (peptidoglycan biosynthesis IV (PWY 6471) and/or peptidoglycan biosynthesis V beta-lactam resistance (PWY 6470)) (e.g., peptidoglycan biosynthesis IV (PWY 6471)). In certain embodiments, a pharmaceutical composition of the present disclosure can include bacteria comprising a gene involved in peptidoglycan biosynthesis IV (e.g., PWY 6471).

In certain embodiments, a pharmaceutical composition of the present disclosure can include bacteria comprising one or more genes involved in Biotin biosynthesis II (e.g., PWY 5005), CMP-legionaminate biosynthesis (e.g., PWY 6749), chondroitin sulfate degradation I, bacterial (e.g., PWY 6572), superpathway of polyamine biosynthesis II (e.g., Polyaminsyn 3 PWY), TCA cycle, prokaryotic (e.g., TCA), pyrimidine deoxyribonucleotides de novo biosynthesis (e.g., PWY 7184), GDP-mannose biosynthesis (e.g., PWY 5659), NAD salvage pathway II (e.g., NAD biosynthesis II), CMP 3-deoxy-D-manno-octulosonate biosynthesis (e.g., PWY 1269), superpathway of pyrimidine nucleobases salvage (e.g., PWY 7208), superpathway of thiamine diphosphate biosynthesis II (e.g., PWY 6895), polyisoprenoid biosynthesis, E. coli (e.g., polyisoprensyn PWY), thiamine diphosphate salvage II (e.g., PWY 6897), superpathway of phospholipid biosynthesis I, bacteria (e.g., Phoslipsyn PWY), L-arginine biosynthesis III, via N-acetyl-L-citrulline (e.g., PWY 5154), superpathway of purine nucleotides de novo synthesis (e.g., PWY 841), superpathway of L-aspartate and L-asparagine biosynthesis (e.g., ASPASN PWY), flavin biosynthesis III, fungi (e.g., PWY 6168), Pre QO biosynthesis (e.g., PWY 6703), L-histidine degradation III (e.g., PWY 6168), superpathway of thiamine diphosphate biosynthesis III, eukaryotes (e.g., THISYNARA PWY), L-ornithine biosynthesis (e.g., Arginine Syn4 PWY), 6-hydroxymethyl-dihydropterin diphosphate biosynthesis I (e.g., PWY 6147), 6-hydroxymethyl-dihydropterin diphosphate biosynthesis III, chlamydia (e.g., PWY 7539) and/or dTDP-β-L-rhamnose biosynthesis (e.g., DTDPRHAMSYN PWY).

In certain embodiments, a pharmaceutical composition of the present disclosure can further include at least one other agent, such as a stabilizing compound or additional therapeutic agent, for example, a probiotic, prebiotic and/or postbiotic.

In certain embodiments, a pharmaceutical composition can further include an antibiotic. In certain embodiments, the antibiotic present in the pharmaceutical composition does not target the bacteria present in the composition. In certain embodiments, the antibiotic is selected from clindamycin, cephalexin, tobramycin, cefpodoxime, atovaquone, ampicillin/sulbactam, cefuroxime and cefazolin, levofloxacin, cefepime, ciprofloxacin and vancomycin. In certain embodiments, the antibiotic is not piperacillin-tazobactam, imipenem-cilastatin and meropenem.

In certain embodiments, the pharmaceutical composition can be administered in any sterile, biocompatible pharmaceutical carrier, including, but not limited to, saline, buffered saline, dextrose, glycerol, polyethylene glycol, and water. In certain embodiments, the pharmaceutical composition can be in a liquid or lyophilized or freeze-dried form. In certain embodiments, a formulation includes a diluent (for example, a buffer such as Tris, citrate, acetate or phosphate buffers) having suitable pH values and ionic strengths, solubilizer such as polysorbate (e.g., Tween®), carriers such as human serum albumin or gelatin.

In certain embodiments, a pharmaceutical composition of the present disclosure can further include a preservative. In certain embodiments, the preservative does not affect viability of the organisms in the pharmaceutical composition. Non-limiting examples of preservatives include thimerosal, parabens, benzylalconium chloride or benzyl alcohol, antioxidants such as ascorbic acid or sodium metabisulfite, and other components such as lysine or glycine. Selection of a particular composition can depend upon a number of factors, including the condition being treated, the route of administration and the pharmacokinetic parameters desired. A more extensive survey of components suitable for pharmaceutical compositions is found in Remington's Pharmaceutical Sciences, 18th ed. A. R. Gennaro, ed. Mack, Easton, PA (1980).

In certain embodiments, the pharmaceutical compositions of the present disclosure can be used for treating a subject having a cancer, increasing the likelihood of cancer survival in a subject after CAR T cell therapy, decreasing the likelihood of a CAR T cell associated toxicity in a subject or a combination thereof.

The route of administration eventually chosen will depend upon a number of factors and can be ascertained by one skilled in the art. In certain embodiments, the pharmaceutical compositions of the present disclosure can be formulated using pharmaceutically acceptable carriers well known in the art in dosages suitable for oral, nasogastric or rectal administration. Such carriers enable the pharmaceutical compositions to be formulated as tablets, pills, capsules, liquids, gels, syrups, slurries, suspensions and the like, for oral, rectal or nasal ingestion by a patient to be treated. In certain embodiments, the formulation includes a capsule or tablet formulated for gastrointestinal delivery, e.g., an enteric coated capsule or pill.

In certain embodiments, pharmaceutical compositions for use in the present disclosure can include pharmaceutical compositions where the active ingredients, e.g., therapeutic bacteria, are contained in an effective amount to achieve the intended purpose. The amount will vary from one individual to another and will depend upon a number of factors, including the intestinal microbiota of the subject, whether cells for modification have been collected from the patient, whether modified T cells have been administered to the patient, the type and dose of cancer treated by the CAR T cell therapy, the results of any methods described herein to assess the risk of the patient exhibiting a poor response to the CAR T cell therapy or achieving a partial response to complete response to the CAR T cell therapy, the chances of the patient developing toxicity, including severe toxicity, and the overall physical condition of the patient.

In certain embodiments, the compositions of the present disclosure can be administered for therapeutic use including prophylactic treatments. For example, but not by way of limitation, pharmaceutical compositions of the present disclosure can be administered in an amount sufficient to reduce the risk of a poor response to a CAR T cell therapy, to increase the response to a CAR T cell therapy and/or to increase the likelihood of survival, e.g., cancer survival, in a subject. In certain embodiments, the pharmaceutical compositions of the present disclosure can be administered in an amount sufficient for reducing the risk of CAR T cell therapy toxicity in a subject. Dosages for any one patient depends upon many factors, including stage of the disease or condition, the severity of the disease or condition, the patient's size, body surface area, age, the particular compound to be administered, sex, time and route of administration, general health, and interaction with other drugs being concurrently administered.

A therapeutic bacteria can be administered to a patient alone, or in combination with one or more other drugs, nucleotide sequences, lifestyle changes, etc. used in combination with a CAR T cell therapy, including those designed to treat or reduce the risk of toxicity, including severe toxicity, and/or in pharmaceutical compositions where it is mixed with excipient(s) or other pharmaceutically acceptable carriers.

Single or multiple administrations of formulations can be given depending on the dosage and frequency as required and tolerated by the patient. The formulations can provide a sufficient quantity of active agent to increase the probability of survival following a CAR T cell therapy or to increase the chance of a response to a CAR T cell therapy.

### V. Methods of Treatments

The present disclosure provides methods of treating subjects having cancer. In certain embodiments, the present disclosure provides a method of treating a subject having cancer to increase the likelihood of cancer survival in the subject after CAR T cell therapy. In certain embodiments, the present disclosure provides a method for treating a subject having cancer to decrease the likelihood of a CAR T cell associated toxicity in a subject.

In certain embodiments, the methods disclosed herein, include administering to the subject, at least one presently disclosed therapeutic bacteria or spores thereof or a pharmaceutical composition comprising thereof. In certain embodiments, the methods further comprise administering to the subject a CAR T cell therapy. In certain embodiments, the therapeutic bacteria or spores thereof, or the pharmaceutical composition comprising thereof, is administered to the subject prior to or during the CAR T cell therapy.

In certain embodiments, methods of the present disclosure can further include the administration of an antibiotic. For example, but not by way of limitation, a method of the present disclosure can include the administration of an antibiotic followed by the administration of a CAR T cell therapy. In certain embodiments, the antibiotic is not piperacillin-tazobactam, imipenem-cilastatin or meropenem.

Patients in need of such treatment or compositions include patients who are receiving or are being considered for or can receive CAR T cell therapy. Such patients typically include patients with certain cancers. Non-limiting examples of such cancers are disclosed herein. In certain embodiments, the cancer is selected from the group consisting of acute lymphoblastic leukemia, acute myelogenous leukemia, biliary cancer, breast cancer, cervical cancer, chronic lymphocytic leukemia, chronic myelogenous leukemia, colorectal cancer, endometrial cancer, esophageal, gastric, head and neck cancer, Hodgkin's lymphoma, lung cancer, medullary thyroid cancer, non-Hodgkin's lymphoma, multiple myeloma, renal cancer, ovarian cancer, pancreatic cancer, glioma, melanoma, liver cancer, prostate cancer, and urinary bladder cancer, CD19 malignancies, and other B cell-related or hematologic malignancies. In certain embodiments, the cancer is an ovarian cancer, a multiple myeloma, or a B-cell malignancy and any combinations thereof. Non-limiting examples of B-cell malignancies include acute lymphoblastic leukemia (ALL), chronic lymphoblastic leukemia (CLL) or non-Hodgkin lymphoma (NHL)). In certain embodiments, the cancer is ALL. In certain embodiments, the cancer is NHL. In certain embodiments, the patient is a human subject.

In certain embodiments, such subjects can, in particular, include those identified using diagnostic methods disclosed herein to have a decreased likelihood of cancer survival following CAR T cell therapy or an increased likelihood of CAR T cell associated toxicity, or a combination thereof. In certain embodiments, the administration of a therapeutic bacterium disclosed herein or a pharmaceutical composition thereof can increase the likelihood of cancer survival following CAR T cell therapy or decrease the likelihood of CAR T cell associated toxicity, or a combination thereof.

In certain embodiments, such subjects can include those identified using diagnostic methods disclosed herein to have an increased likelihood of cancer survival following CAR T cell therapy or a decreased likelihood of CAR T cell associated toxicity, or a combination thereof. In certain embodiments, the administration of a therapeutic bacterium disclosed herein or a pharmaceutical composition thereof can further increase the likelihood of cancer survival following CAR T cell therapy or further decrease the likelihood of CAR T cell associated toxicity, or a combination thereof.

In certain embodiments, the present disclosure provides for a method of increasing the likelihood of cancer survival following CAR T cell therapy or decreasing the likelihood of CAR T cell associated toxicity or a combination thereof, by administering, to a subject in need of such treatment, an effective amount of at least one therapeutic bacteria or a pharmaceutical composition thereof. Non-limiting examples of therapeutic bacteria that can be administered to a subject in need thereof are disclosed in Section III and pharmaceutical compositions that can be administered to a subject in need thereof are disclosed in Section IV.

In certain embodiments, a method of treating a subject having cancer can include (a) identifying the subject as having a decreased likelihood of cancer survival following CAR T cell therapy, e.g., determined by any one of the methods disclosed herein, (b) administering an effective amount of a therapeutic bacterium disclosed herein or a pharmaceutical composition thereof and (c) administering a CAR T cell therapy to the subject. Non-limiting examples of pharmaceutical compositions are disclosed in Section IV. In certain embodiments, the pharmaceutical composition can comprise one or more bacterium selected from Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti, Faecalibacterium prausnitzii or a combination thereof. In certain embodiments, the pharmaceutical composition can comprise one or more bacterium comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti and/or Faecalibacterium prausnitzii. In certain embodiments, the pharmaceutical composition can include Blautia luti and/or Faecalibacterium prausnitzii. In certain embodiments, the pharmaceutical composition can include Ruminococcus gnavus and/or Ruminococcus bromii.

In certain embodiments, a method of treating a subject having cancer can include (a) identifying the subject as having an increased likelihood of CAR T cell associated toxicity, e.g., determined by any one of the methods disclosed herein, (b) administering a therapeutic bacterium disclosed herein or a pharmaceutical composition thereof and (c) administering a CAR T cell therapy to the subject. Non-limiting examples of pharmaceutical compositions are disclosed in Section IV. In certain embodiments, the pharmaceutical composition can comprise one or more bacterium selected from Streptococcus oralis, Lutispora thermophila, Agathobaculum butyriciproducens, Lactobacillus salivarius, Schaalia odontolytica, Blautia luti, Faecalibacterium prausnitzii or a combination thereof. In certain embodiments, the pharmaceutical composition can comprise one or more bacterium comprising a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica, Blautia luti and/or Faecalibacterium prausnitzii. In certain embodiments, the pharmaceutical composition can include Blautia luti and/or Faecalibacterium prausnitzii.

In certain embodiments, a method of treating a subject having cancer can include (a) identifying the subject as having an increased likelihood of cancer survival following CAR T cell therapy, e.g., determined by any one of the methods disclosed herein, and (b) administering a CAR T cell therapy to the subject. In certain embodiments, the method can further include the administration of a therapeutic bacterium disclosed herein or a pharmaceutical composition thereof prior to CAR T cell therapy.

In certain embodiments, a method of treating a subject having cancer can include (a) identifying the subject as having a decreased likelihood of CAR T cell associated toxicity, e.g., determined by any one of the methods disclosed herein, and (b) administering a CAR T cell therapy to the subject. In certain embodiments, the method can further include the administration of a therapeutic bacterium disclosed herein or a pharmaceutical composition thereof prior to CAR T cell therapy.

In certain embodiments, a method of the present disclosure can further include administering an effective amount of a prebiotic. In certain embodiments, the prebiotic can be administered separately from the therapeutic bacteria and can promote the growth, proliferation and/or survival of at least one therapeutic bacteria. In certain embodiments, the prebiotic can include one or more agents, for example, a nutritional supplement, that increases growth and survival of at least one therapeutic bacteria. The prebiotic can include one or more of poorly-absorbed complex carbohydrates, oligosaccharides, inulin-type fructans or arabinoxylans.

In certain embodiments, a method of the present disclosure can further administering an effective amount of a postbiotic. The postbiotic can be administered separately from the therapeutic bacteria.

The present disclosure provides the use of any composition described herein, including the use of any therapeutic bacteria described herein for increasing the likelihood of cancer survival following CAR T cell therapy or decreasing the likelihood of CAR T cell associated toxicity or a combination thereof in a subject. The use can be further characterized by aspects of the methods described above and elsewhere herein.

### VI. Kits

The presently disclosed subject matter provides for kits for performing any of the methods disclosed herein or providing any of the therapeutic bacteria disclosed herein or pharmaceutical compositions thereof.

In certain embodiments, a kit of the present disclosure is for use in diagnosing a subject's likelihood of cancer survival following CAR T cell therapy or likelihood of CAR T cell associated toxicity. In certain embodiments, a kit of the present disclosure is for use in treating a subject having cancer, e.g., for increasing the likelihood of cancer survival following CAR T cell therapy or decreasing the likelihood of CAR T cell associated toxicity or a combination thereof.

In certain embodiments, a kit of the present disclosure can include an agent for determining whether a sample (e.g., a feces sample or an intestinal content sample) of a subject contains an increased or decreased level of a bacterium or spores thereof, or a bacterial gene as compared to a reference level.

An increased or decreased level of the bacterium or spores thereof or the bacterial gene is determined with respect to a reference bacterium or spores thereof level or a reference bacterial gene level. In certain embodiments, the level (e.g., the measured level and the reference level) can be based on a relative abundance in the intestinal microbiome. For instance, the level can represent a percentage of the bacterium or spores thereof of all the bacteria or spores thereof in the intestinal microbiome. The level can also be an absolute number.

In certain embodiments, the agent is used for determining the level of a bacterium in the sample of the subject, where the bacterium is selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Hungatella effluvii, Coprobacillus cateniformis, Atopobium parvulum, Faecalicoccus acidiformans, Lactococcus lactis, Anaerobacterium chartisolvens, Atopobiaceae, Clostridium innocuum, Bacteroides thetaiotamicron, Streptococcus salivarius, Oscillibacter ruminantium, Eubacterium ramulus, Streptococcus gordonii, Caproiciproducens galactitolivorans, Clostridium hylemonae, Streptococcus oralis, Lutispora thermophila, Agathobaculum butyriciproducens, Lactobacillus salivarius, Schaalia odontolytica, Ruminococcus bromii, Bifidobacterium breve, Blautia luti, Longicatena caecimuris, Escherichia coli, Faecalibacterium prausnitzii and any combinations thereof.

In certain embodiments, the agent is used for determining a bacterial gene in a sample of the subject, where the bacterial gene is selected from the group consisting of genes involved in purine nucleosides degradation (e.g., PWY 1296), chorismate biosynthesis (e.g., ARO PWY), antigen building blocks biosynthesis (e.g., O Antigen Pathway), Calvin Benson Bassham cycle (e.g., Calvin PWY), peptidoglycan biosynthesis IV Enterococcus faecium (e.g., PWY 6471), adenine and adenosine salvage III (e.g., PWY 6609), superpathway of aromatic amino acid biosynthesis (e.g., COMPLETE ARO PWY), UDP N-acetyl D glucosamine biosynthesis (e.g., UDPNAGSYN PWY), chorismate biosynthesis from 3-dehydroguinate (e.g., PWY 6163), L-methionine biosynthesis III (e.g., HSERMETANA PWY), peptidoglycan biosynthesis V beta-lactam resistance (e.g., PWY 6470), superpathway of pyrimidine ribonucleosides salvage (e.g., PWY 7196), heterolactic fermentation (e.g., P122 PWY), allantoin degradation glyoxylate II (e.g., PWY 5692), anaerobic energy metabolism (e.g., PWY 7384), superpathway of allantoin degradation (e.g., URDEGR PWY), superpathway of anaerobic energy metabolism (e.g., PWY 7389), superpathway of unsaturated fatty acid biosynthesis (e.g., PWY 6284), enterobactin biosynthesis (e.g., ENTBACSYN PWY), NAD salvage pathway III (e.g., NAD Biosynthesis II), superpathway of fatty acid biosynthesis (e.g., PWY 6285), pyrimidine deoxyribonucleotide biosynthesis from CTP (e.g., PWY 7210), superpathway of mycolate biosynthesis (e.g., PWY 6113), NAD NADH phosphorylation and dephosphorylation (e.g., PWY 5083), palmitate biosynthesis II bacteria and plants (e.g., PWY 5971), biotin synthesis I (e.g., BIOTIN BIOSYNTHESIS PWY), CMP 3-deoxy-D-manno-octulosonate biosynthesis (e.g., PWY 1269), lipid IVA biosynthesis (e.g., NAGLIPASYN) and/or peptidoglycan biosynthesis (peptidoglycan biosynthesis IV Enterococcus faecium (e.g., PWY 6471) and any combinations thereof.

In certain embodiments, the agent is used for determining a bacterial gene in a sample of the subject, where the bacterial gene is selected from the group consisting of genes involved in pentose phosphate pathway non-oxidative branch (e.g., nonoxipent PWY), L-isoleucine biosynthesis (e.g., PWY 5104), pyruvate fermentation to acetate and lactate II (PWY 5100), superpathway of glycerol degradation to 1,3-propanediol (e.g., GOLPDLCAT PWY), formaldehyde assimilation III, dihydroxyacetone cycle (e.g., PWY P185), pyrimidine deoxyribonucleotides de novo biosynthesis IV (PWY 7198), acetyl-CoA fermentation to butanoate II (e.g., PWY 5676), glycerol degradation to butanol (e.g., PWY 7003), superpathway of (R,R)-butanediol biosynthesis (e.g., PWY P125), Biotin biosynthesis II (e.g., PWY 5005), CMP-legionaminate biosynthesis (e.g., PWY 6749), chondroitin sulfate degradation I, bacterial (e.g., PWY 6572), superpathway of polyamine biosynthesis II (e.g., Polyaminsyn 3 PWY), TCA cycle, prokaryotic (e.g., TCA), pyrimidine deoxyribonucleotides de novo biosynthesis (e.g., PWY 7184), GDP-mannose biosynthesis (e.g., PWY 5659), NAD salvage pathway II (e.g., NAD biosynthesis II), CMP 3-deoxy-D-manno-octulosonate biosynthesis (e.g., PWY 1269), superpathway of pyrimidine nucleobases salvage (e.g., PWY 7208), superpathway of thiamine diphosphate biosynthesis II (e.g., PWY 6895), polyisoprenoid biosynthesis, E. coli (e.g., polyisoprensyn PWY), thiamine diphosphate salvage II (e.g., PWY 6897), superpathway of phospholipid biosynthesis I, bacteria (e.g., Phoslipsyn PWY), L-arginine biosynthesis III, via N-acetyl-L-citrulline (e.g., PWY 5154), superpathway of purine nucleotides de novo synthesis (e.g., PWY 841), superpathway of L-aspartate and L-asparagine biosynthesis (e.g., ASPASN PWY), flavin biosynthesis III, fungi (e.g., PWY 6168), Pre QO biosynthesis (e.g., PWY 6703), L-histidine degradation III (e.g., PWY 6168), superpathway of thiamine diphosphate biosynthesis III, eukaryotes (e.g., THISYNARA PWY), L-ornithine biosynthesis (e.g., Arginine Syn4 PWY), 6-hydroxymethyl-dihydropterin diphosphate biosynthesis I (e.g., PWY 6147), 6-hydroxymethyl-dihydropterin diphosphate biosynthesis III, chlamydia (e.g., PWY 7539) and/or dTDP-β-L-rhamnose biosynthesis (e.g., DTDPRHAMSYN PWY), CMP-legionaminate biosynthesis (e.g., PWY 6749) and/or Pre QO biosynthesis (e.g., PWY 6703) and any combinations thereof.

In certain embodiments, the agent can include nucleic acid primers specific for said bacteria or genes, such as nucleic acid primers are specific for 16S rRNA gene sequencing.

The presently disclosed subject matter also provides for kits for treating a subject who has received or can receive CAR T cell therapy. For example, but not by way of limitation, a kit of the present disclosure can be used for treating a subject that has been identified as having a decreased likelihood of cancer survival and/or an increased likelihood of CAR T cell associated toxicity. Such kits can include one or more therapeutic bacteria or pharmaceutical compositions as described herein (e.g., disclosed in Sections III and IV).

In certain embodiments, the kit can include instructions for administering the therapeutic bacteria or pharmaceutical compositions thereof. The instructions can include information about the use of the therapeutic bacterial or pharmaceutical compositions thereof in conjunction with CAR T cell therapy. The instructions can include at least one of the following: description of the therapeutic bacteria or composition; dosage schedule and administration; precautions; warnings; indications; counter-indications; over dosage information; adverse reactions; animal pharmacology; clinical studies; and/or references. The instructions can be printed directly on a container (when present) containing the therapeutic bacteria or composition, or as a label applied to the container, or as a separate sheet, pamphlet, card, or folder supplied in or with the container.

In certain embodiments, a kit of the present disclosure can include both components for diagnosing whether a subject has an increased/decreased likelihood of cancer survival following CAR T cell therapy or an increased/decreased likelihood of CAR T cell associated toxicity receiving or considered for CAR T cell therapy, and components for treating a subject who has or can receive or can CAR T cell therapy, e.g., one or more therapeutic bacteria. The kit can include instructions for administering components for treating the subject based upon results obtained using the components for diagnosing the subject.

### VII. Exemplary Embodiments

A. The present disclosure provides a pharmaceutical composition comprising an effective amount of (a) a therapeutic bacterium or a spore thereof selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Streptococcus oralis, Lutispora thermophila, Agathobaculum butyriciproducens, Lactobacillus salivarius, Schaalia odontolytica, Longicatena caecimuris, Bifidobacterium breve, Ruminococcus bromii, Blautia luti, Faecalibacterium prausnitzii or a combination thereof; and/or (b) a bacterium or a spore thereof comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Streptococcus oralis, Lutispora thermophila, Agathobaculum butyriciproducens, Lactobacillus salivarius, Schaalia odontolytica, Longicatena caecimuris, Bifidobacterium breve, Ruminococcus bromii, Blautia luti, Faecalibacterium prausnitzii or a combination thereof.

A1. The pharmaceutical composition of A, further comprising a biocompatible pharmaceutical carrier.

A2. The pharmaceutical composition of A-A1, wherein the pharmaceutical composition is formulated for oral, nasogastric, rectal, percutaneous, orogastric tube administration.

A3. The pharmaceutical composition of any one of A-A2 further comprising a probiotic bacteria, a probiotic yeast, a prebiotic, a postbiotic, an antibiotic or a combination thereof.

A4. The pharmaceutical composition of any one of A-A3, wherein the pharmaceutical composition is in a form of a liquid, a suspension, a dried powder, a tablet, a capsule, a food product or a combination thereof.

A5. The pharmaceutical composition of any one of A-A4, wherein the bacterium or spore thereof is a recombinant bacterium or a progeny thereof.

A6. The pharmaceutical composition of any one of A-A5, comprising (a) a therapeutic bacterium or a spore thereof selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti, Faecalibacterium prausnitzii or a combination thereof; and/or (b) a bacterium or a spore thereof comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti, Faecalibacterium prausnitzii or a combination thereof.

A7. The pharmaceutical composition of A6, comprising (a) a therapeutic bacterium or a spore thereof selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans or a combination thereof; and/or (b) a bacterium or a spore thereof comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans or a combination thereof.

A8. The pharmaceutical composition of A6, comprising (a) a therapeutic bacterium or a spore thereof selected from the group consisting of Ruminococcus bromii, Longicatena caecimuris, Bifidobacterium breve, Blautia luti, Faecalibacterium prausnitzii or a combination thereof; and/or (b) a bacterium or a spore thereof comprising a 16S rRNA gene sequence having at least 94.5%, 95%, 97%, 98%, 98.7% or 99% sequence identity to the 16S rRNA gene sequence of Ruminococcus bromii, Longicatena caecimuris, Bifidobacterium breve, Blautia luti, Faecalibacterium prausnitzii or a combination thereof.

A9. The pharmaceutical composition of any one of claims A-A6, comprising (a) a therapeutic bacterium or a spore thereof selected from the group consisting of Streptococcus oralis, Lutispora thermophila, Agathobaculum butyriciproducens, Lactobacillus salivarius, Blautia luti, Faecalibacterium prausnitzii or a combination thereof; and/or (b) a bacterium or a spore thereof comprising a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Blautia luti, Faecalibacterium prausnitzii or a combination thereof.

A10. The pharmaceutical composition of A9, comprising (a) a therapeutic bacterium or a spore thereof selected from the group consisting of Streptococcus oralis, Lutispora thermophila, Agathobaculum butyriciproducens, Lactobacillus salivarius, Schaalia odontolytica or a combination thereof; and/or (b) a bacterium or a spore thereof comprising a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica or a combination thereof.

A11. The pharmaceutical composition of A9, comprising (a) a therapeutic bacterium or a spore thereof selected from the group consisting of Blautia luti, Faecalibacterium prausnitzii or a combination thereof; and/or (b) a bacterium or a spore thereof comprising a 16S rRNA gene sequence having at least 94.5%, 95%, 97%, 98%, 98.7% or 99% sequence identity to the 16S rRNA gene sequence of Blautia luti, Faecalibacterium prausnitzii or a combination thereof.

A12. The pharmaceutical composition of any one of A6-A8, wherein the effective amount of the bacterium or spore thereof increases the likelihood of cancer survival in a subject administered the pharmaceutical composition.

A13. The pharmaceutical composition of any one of A9-A11, wherein the effective amount of the bacterium or spore thereof decreases the likelihood of CAR T cell associated toxicity in a subject administered the pharmaceutical composition.

B. The present disclosure provides a method for identifying a subject having a cancer as having a decreased likelihood of cancer survival following a CAR T cell therapy that includes:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a patient that did not exhibit cancer survival; and
(c) identifying the subject as having a decreased likelihood of cancer survival if the level of the diagnostic bacterium or spore thereof is higher than the reference level;
wherein the bacterium or spore thereof is selected from the group consisting of Hungatella effluvii, Coprobacillus cateniformis, Atopobium parvulum, Faecalicoccus acidiformans, Lactococcus lactis, Anaerobacterium chartisolvens, Atopobiaceae Clostridium innocuum, Bacteroides thetaiotaomicron, a species of the Veillonellaceae family and a combination thereof; or the bacterium or spore thereof comprises a 16S rRNA gene sequence having at least 94.5%, 95%, 97%, 98%, 98.7% or 99% sequence identity to the 16S rRNA gene sequence of Hungatella effluvii, Coprobacillus cateniformis, Atopobium parvulum, Faecalicoccus acidiformans, Lactococcus lactis, Anaerobacterium chartisolvens, Atopobiaceae Clostridium innocuum, Bacteroides thetaiotaomicron, a species of the Veillonellaceae family or a combination thereof

B1. The method of B, wherein the bacterium or spore thereof is selected from the group consisting of Hungatella effluvii, Coprobacillus cateniformis, Atopobium parvulum, Faecalicoccus acidiformans, Lactococcus lactis, Anaerobacterium chartisolvens, Atopobiaceae Clostridium innocuum, Bacteroides thetaiotaomicron and a combination thereof; or the bacterium or spore thereof comprises a 16S rRNA gene sequence having at least 94.5%, 95%, 97%, 98%, 98.7% or 99% sequence identity to the 16S rRNA gene sequence of Hungatella effluvii, Coprobacillus cateniformis, Atopobium parvulum, Faecalicoccus acidiformans, Lactococcus lactis, Anaerobacterium chartisolvens, Atopobiaceae Clostridium innocuum, Bacteroides thetaiotaomicron or a combination thereof.

B2. The method of B, wherein the bacterium or spore thereof is a species of the Veillonellaceae family or is a bacterium or spore thereof comprising a 16S rRNA gene sequence having at least 94.5%, 95%, 97%, 98%, 98.7% or 99% sequence identity to the 16S rRNA gene sequence of a species of the Veillonellaceae family.

B3. The method of any one of B-B2, further comprising treating the subject identified as having a decreased likelihood of exhibiting cancer survival with the pharmaceutical composition of any one of A-A8.

C. The present disclosure provides a method for identifying a subject having a cancer as having an increased likelihood of cancer survival following a CAR T cell therapy comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a patient exhibited cancer survival; and
(c) identifying the subject as having increased likelihood of cancer survival if the level of the diagnostic bacterium or spore thereof is higher than the reference level; wherein the bacterium or spore thereof is selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Blautia luti, Faecalibacterium prausnitzii or a combination thereof.

C1. The method of C, wherein the bacterium or spore thereof is selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans and a combination thereof; or the bacterium or spore thereof comprises a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans or a combination thereof.

C2. The method of C, wherein the bacterium or spore thereof is selected from the group consisting of Longicatena caecimuris, Bifidobacterium breve, Escherichia coli, Ruminococcus bromii, Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Longicatena caecimuris, Bifidobacterium breve, Escherichia coli, Ruminococcus bromii, Blautia luti, Faecalibacterium prausnitzii or a combination thereof.

D. The present disclosure provides a method for identifying a subject having a cancer as having an increased likelihood of cancer survival and/or a decreased likelihood of CAR T cell associated toxicity comprising:
(a) determining a level of a bacterial gene in a sample of the subject;
(b) comparing the level of the bacterial gene to a reference bacterial gene level, wherein the reference level is the level of the bacterial gene in a patient that exhibited cancer survival and/or in a patient that did not exhibit CAR T cell associated toxicity; and
(c) identifying the subject as having an increased likelihood of cancer survival and/or a decreased likelihood of CAR T cell associated toxicity if the level of the bacterial gene is higher than the reference bacterial gene level;
wherein the bacterial gene is a gene involved in aromatic amino acid biosynthesis and/or peptidoglycan biosynthesis and/or dTDP-β-L-rhamnose biosynthesis (e.g., DTDPRHAMSYN PWY).

D1. The method of any one of B-D, further comprising treating the subject with a CAR T cell therapy.

D2. The method of any one of B-D1, wherein cancer survival is the survival of the subject at least about 100 days following a CAR T cell therapy.

E. The present disclosure provides a method for identifying a subject having a cancer as having an increased likelihood of exhibiting a CAR T cell associated toxicity comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a patient that exhibited a CAR T cell associated toxicity; and
(c) identifying the subject as having an increased likelihood of exhibiting a CAR T cell associated toxicity if the level of the bacterium or spore thereof is higher than the reference level;
wherein the bacterium or spore thereof is selected from the group consisting of Streptococcus salivarius, Oscillibacter ruminantium, Eubacterium ramulus, Streptococcus gordonii, Caprocicproducens galactitolivorans, Clostridium hylemonae and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus salivarius, Oscillibacter ruminantium, Eubacterium ramulus, Streptococcus gordonii, Caprocicproducens galactitolivorans, Clostridium hylemonae and a combination thereof.

E1. The method of E, further comprising treating the subject identified as having an increased likelihood of exhibiting a CAR T cell associated toxicity with the pharmaceutical composition of any one of A-A5 and A9-A11.

E2. The method of E or E1, further comprising treating the subject with a CAR T cell therapy.

F. The present disclosure provides a method for identifying a subject having a cancer as having a decreased likelihood to exhibit cancer survival and/or an increased likelihood to exhibit a CAR T cell associated toxicity comprising:
(a) determining if the subject has been administered an antibiotic; and
(b) identifying the subject as having an increased likelihood of a CAR T cell associated toxicity and/or a decreased likelihood of cancer survival if the subject has been administered piperacillin-tazobactam, imipenem-cilastatin, meropenem or a combination thereof.

F1. The method of F, wherein the antibiotic is administered less than about 4 weeks prior to the initiation of a CAR T cell therapy.

F2. The method of any one of claims E-F1, wherein the CAR T cell associated toxicity is cytokine release syndrome (CRS) and/or immune effector cell-associated neurotoxicity syndrome (ICANS).

G. The present disclosure provides a method for treating a subject having a cancer with a CAR T cell therapy, comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a subject that exhibited cancer survival;
(c) identifying the subject as having an increased likelihood of cancer survival if the level of the bacterium or spore thereof is higher than the reference level; and
(d) treating the subject identified as having an increased likelihood of cancer survival with a CAR T cell therapy,
wherein the bacterium or spore thereof is selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Blautia luti, Longicatena caecimuris, Bifidobacterium breve, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Blautia luti, Longicatena caecimuris, Bifidobacterium breve or Faecalibacterium prausnitzii.

G1. The method of G, wherein the bacterium or spore thereof is selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans and a combination thereof; or the bacterium or spore thereof comprises a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis or Clostridium methoxybenzovorans.

G2. The method of G, wherein the bacterium or spore thereof is selected from the group consisting of Ruminococcus bromii, Blautia luti, Longicatena caecimuris, Bifidobacterium breve, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Ruminococcus bromii, Blautia luti, Longicatena caecimuris, Bifidobacterium breve or Faecalibacterium prausnitzii.

H. The present disclosure provides a method for treating a subject having a cancer with a CAR T cell therapy, comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a subject that did not exhibit cancer survival;
(c) identifying the subject as having a decreased likelihood of cancer survival if the level of the bacterium or spore thereof is higher than the reference level; and
(d) treating the subject identified as having a decreased likelihood of cancer survival with a pharmaceutical composition comprising one or more therapeutic bacteria and a CAR T cell therapy,
wherein the bacterium or spore thereof is selected from the group consisting of Hungatella effluvii, Coprobacillus cateniformis, Atopobium parvulum, Faecalicoccus acidiformans, Lactococcus lactis, Anaerobacterium chartisolvens, Clostridium innocuum, Bacteroides thetaiotaomicron, a species of the Veillonellaceae family and a combination thereof; or the bacterium or spore thereof comprises a 16S rRNA gene sequence having at least 94.5%, 95%, 97%, 98%, 98.7% or 99% sequence identity to the 16S rRNA gene sequence of Hungatella effluvii, Coprobacillus cateniformis, Atopobium parvulum, Faecalicoccus acidiformans, Lactococcus lactis, Anaerobacterium chartisolvens, Clostridium innocuum, Bacteroides thetaiotaomicron, a species of the Veillonellaceae family and a combination thereof,
wherein the therapeutic bacteria comprises one or more of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti and/or Faecalibacterium prausnitzii or a bacteria comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti and/or Faecalibacterium prausnitzii.

H1. The method of any one of G-G2 and H, wherein cancer survival is the survival of the subject at least about 100 day following a CAR T cell therapy.

I. The present disclosure provides a method for treating a subject having a cancer with a CAR T cell therapy, comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a subject that did not exhibit a CAR T cell associated toxicity;
(c) identifying the subject as having a decreased likelihood of exhibiting a CAR T cell associated toxicity if the level of the bacterium or spore thereof is higher than the reference level; and
(d) treating the subject identified as having a decreased likelihood of exhibiting a CAR T cell associated toxicity with a CAR T cell therapy,
wherein the bacterium or spore thereof is selected from the group consisting of Streptococcus oralis, Lutispora thermophila, Agathobaculum butyriciproducens, Lactobacillus salivarius, Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Blautia luti and/or Faecalibacterium prausnitzii.

I1. The method of I, wherein the bacterium or spore thereof is selected from the group consisting of Streptococcus oralis, Lutispora thermophila, Agathobaculum butyriciproducens, Lactobacillus salivarius and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens and/or Lactobacillus salivarius.

I2. The method of I, wherein the bacterium or spore thereof is selected from the group consisting of Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Blautia luti and/or Faecalibacterium prausnitzii.

J. The present disclosure provides a method for treating a subject having a cancer with a CAR T cell therapy, comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a subject that exhibited CAR T cell associated toxicity;
(c) identifying the subject as having an increased likelihood of exhibiting a CAR T cell associated toxicity if the level of the bacterium or spore thereof is higher than the reference level; and
(d) treating the subject identified as having an increased likelihood of exhibiting a CAR T cell associated toxicity with a pharmaceutical composition comprising one or more therapeutic bacteria and a CAR T cell therapy,
wherein the bacterium or spore thereof is selected from the group consisting of Streptococcus salivarius, Oscillibacter ruminantium, Eubacterium ramulus, Streptococcus gordonii, Caprocicproducens galactitolivorans, Clostridium hylemonae and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus salivarius, Oscillibacter ruminantium, Eubacterium ramulus, Streptococcus gordonii, Caprocicproducens galactitolivorans, Clostridium hylemonae or a combination thereof,
wherein the therapeutic bacteria comprises one or more of Streptococcus oralis, Lutispora thermophila, Agathobaculum butyriciproducens, Lactobacillus salivarius, Blautia luti, Faecalibacterium prausnitzii or a bacterium comprising a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Blautia luti, Faecalibacterium prausnitzii or a combination thereof.

J1. The method any one of I-A2 or J, wherein the CAR T cell associated toxicity is cytokine release syndrome (CRS) and/or immune effector cell-associated neurotoxicity syndrome (ICANS).

J2. The method of any one of B-J1, wherein the sample is a fecal sample or an intestinal content sample of the subject.

J3. The method of any one of B-J2, further comprising administering to the subject a chemotherapy, immunotherapy, stem cell therapy, cellular therapy, a probiotic bacteria, a probiotic yeast, a prebiotic, a postbiotic, an antibiotic or a combination thereof.

J4. The method of B-J3, wherein the cancer is selected from the group consisting of acute lymphoblastic leukemia (ALL), chronic lymphoblastic leukemia (CLL) or non-Hodgkin lymphoma (NHL)).

K. The present disclosure provides a kit comprising the pharmaceutical composition of any one of A-A13.

L. The present disclosure provides a kit comprising means for identifying a bacterium or a spore thereof selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Hungatella effluvii, Coprobacillus cateniformis, Atopobium parvulum, Faecalicoccus acidiformans, Lactococcus lactis, Anaerobacterium chartisolvens, Atopobiaceae, Clostridium innocuum, Bacteroides thetaiotamicron, Streptococcus salivarius, Oscillibacter ruminantium, Eubacterium ramulus, Streptococcus gordonii, Caprocicproducens galactitolivorans, Clostridium hylemonae, Streptococcus oralis, Lutispora thermophila, Agathobaculum butyriciproducens, Schaalia odontolytica, Lactobacillus salivarius, Ruminococcus bromii, Bifidobacterium breve, Longicatena caecimuris, Escherichia coli, Blautia luti, Faecalibacterium prausnitzii and a combination thereof.

L1. The kit of K or L, further comprising instructions for treating a subject having a cancer.

L2. The kit of K, L or L1, further comprising instructions for identifying the subject as having an increased likelihood or decreased likelihood of exhibiting cancer survival, wherein the instructions comprise:
(a) determining the level of the bacterium or spore thereof in a sample of the subject;
(b) comparing the level of the bacterium or spores thereof to a reference level of the bacterium or a spore thereof; and
(c) identifying the subject as having an increased likelihood or decreased likelihood of exhibiting cancer survival based on the comparison.

L3. The kit of any one of K and L-L2, further comprising instructions for identifying the subject as having an increased likelihood or decreased likelihood of a CAR T cell associated toxicity, wherein the instructions comprise:
(a) determining the level of the bacterium or spore thereof in a sample of the subject;
(b) comparing the level of the bacterium or spores thereof to a reference level of the bacterium or a spore thereof; and
(c) identifying the subject having an increased likelihood or decreased likelihood of a CAR T cell associated toxicity based on the comparison.

M. The present disclosure provides a use of a pharmaceutical composition of any one of A-A13 for treating a subject having a cancer.

M1. The use of M, wherein the cancer is being treated or will be treated with a CAR T cell therapy.

N. The present disclosure provides a use of a pharmaceutical composition of any one of A6-A8 for increasing the likelihood of cancer survival in a subject.

N1. The use of N, wherein the subject is being treated or will be treated with a CAR T cell therapy.

O. The present disclosure provides a use of a pharmaceutical composition of any one of A9-A11 for decreasing the likelihood of a CAR T cell associated toxicity in a subject.

O1. The use of O, wherein the subject is being treated or will be treated with a CAR T cell therapy.

P. The present disclosure provides a method for identifying a subject having a cancer as having a decreased likelihood of exhibiting a CAR T cell associated toxicity comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a patient that did not exhibit a CAR T cell associated toxicity; and
(c) identifying the subject as having a decreased likelihood of exhibiting a CAR T cell associated toxicity if the level of the bacterium or spore thereof is higher than the reference level;
wherein the bacterium or spore thereof is selected from the group consisting of Streptococcus oralis, Lutispora thermophila, Agathobaculum butyriciproducens, Lactobacillus salivarius, Schaalia odontolytica, Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica, Blautia luti, Faecalibacterium prausnitzii and a combination thereof.

P1. The method of P, wherein the bacterium or spore thereof is selected from the group consisting of Streptococcus oralis, Lutispora thermophila, Agathobaculum butyriciproducens, Lactobacillus salivarius, Schaalia odontolytica and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica and a combination thereof.

P2. The method of P, wherein the bacterium or spore thereof is selected from the group consisting of Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Blautia luti, Faecalibacterium prausnitzii and a combination thereof.

### EXAMPLES

The presently disclosed subject matter will be better understood by reference to the following Example, which is provided as exemplary of the disclosure, and not by way of limitation.

### Example 1: Composition of the Intestinal Microbiota Correlates with Response and Toxicity after CAR T Cell Immunotherapy in Patients with B-Cell Malignancies

Preclinical and clinical studies have previously demonstrated that the intestinal microbiome can regulate T cell immunity in a variety of human diseases. Specifically, the composition of the gut microbiota has been associated with autoimmune diseases, such as inflammatory bowel disease, type-1 diabetes, multiple sclerosis, and rheumatoid arthritis. Other studies have confirmed that the intestinal microbiome can modulate the anti-tumor immune response to chemo and radiation therapy, immune checkpoint blockade, graft-versus-host disease after allogeneic hematopoietic cell transplantation, and adoptive cellular therapy. Furthermore, recent studies have observed that the alteration of the gut microbiota using fecal microbiota transplant improves the anti-tumor response to checkpoint blockade in otherwise refractory melanoma patients. In addition, exposure to antibiotics before treatment with chemotherapy or immune checkpoint blockade is associated with worse outcomes in patients with various cancer types, including lymphoma.

Cellular immunotherapy with chimeric antigen receptor (CAR) T cells has provided new therapeutic options for patients with high-risk hematologic malignancies. Following this therapy, patients can experience disease relapse or CAR-mediated toxicity due to cytokine release syndrome (CRS) or immune effector cell-associated neurotoxicity syndrome (ICANS). The role that the intestinal microbiome can have on these outcomes in a multi-center study was investigated. It was hypothesized that the intestinal microbiome could modulate CAR T cell activity, thereby affecting CAR T immunotherapy efficacy and toxicity. In this Example, the association between antibiotic exposures or fecal microbiome composition with clinical outcomes following CD19 CAR T therapy in patients with B-cell malignancies treated at two institutions, Memorial Sloan Kettering (MSK) and the University of Pennsylvania (Penn), were analyzed.

### Methods

### CAR T Cell Patients.

This study included two primary cohorts consisting of patients receiving CD19-targeted chimeric antigen receptor (CAR) T cell therapy at two Institutions: MSK and Penn. The antibiotic cohort was curated to assess clinical outcomes with respect to antibiotic exposure and included patients (N=228) who received CD19 CAR T cell infusions between 2010 and 2020 (127 from MSK and 101 from Penn), within the following clinical trials (MSK: NCT01044069 (n=55) and Penn: NCT02030834 (n=41), NCT02030847 (n=30), and NCT01029366 (n=6). All other patients were treated with commercial CD19 CAR T cells (n=96). Patients with acute lymphoblastic leukemia (ALL, n=91) and non-Hodgkin lymphoma (NHL, n=137) were included in the analysis. Written informed consent was obtained from all the participants.

The fecal microbiome cohort included patients from whom a baseline fecal sample was collected prior to CD19 CAR T cell infusion. There were 28 patients from MSK and 20 patients from Penn. The study was conducted in accordance with the Declaration of Helsinki.

### Healthy Controls.

The healthy controls consisted of employees at Memorial Sloan Kettering who consented to the collection of a fecal sample under the same IRB-approved protocol used for microbiome analysis in CAR T cell recipients, IRB Protocol No. 06-107, which has a provision for healthy control specimens. Data pertaining to age and sex were not obtained for all participants, but the available data is outlined below. The age range for the healthy controls is 26 to 34 (median 30, 12 values missing) and the group consisted of 44% men (12 values missing). The healthy volunteers were not queried as to whether they had recently taken antibiotics or whether they had any GI disease. They are classified here as healthy controls based on not currently undergoing CAR T therapy.

### Assessment of Clinical Outcomes.

To address differences in institutional practices for assessing treatment response with respect to timing and criteria, tumor responses were broadly classified as either complete response (CR) or no complete response at approximately Day 100 post cell infusion by the treating clinician. This timepoint was assessed given that durable response at 3 months has been associated with long-term response durability. Patients with complete responses at this evaluation were classified as responders, whereas patients with partial response, stable, or progressive disease, were classified as non-complete responders. Disease assessment was based on radiographic or pathologic assessment by the treating clinician. For patients with ALL and NHL, overall survival (OS) was defined as the length of time from CD19 CAR T cell infusion to death. For patients with NHL and ALL, progression-free survival (PFS) was defined as the time from CAR T infusion to the date of progression or death.

CAR-mediated toxicity was classified as any cytokine release syndrome (CRS) or immune effector cell-associated neurotoxicity syndrome (ICANS)/neurotoxicity. At MSK, CRS and ICANS were assessed according to the consensus grading criteria defined by the American Society of Transplantation and Cellular Therapy (ASTCT). Patients treated at Penn were assessed for CRS according to the Penn grading scale. For the antibiotic cohort, different scales were used in between MSK and Penn. This is since patients included in this cohort were treated between 2010 and 2020 in two different institutions. Indeed, at that time there were multiple grading scales for toxicity based on different institutions, due to the relative infancy of CAR T cell immunotherapy development. For this reason, the toxicity grading (0 to 5) was not reported for the antibiotic cohort, but we only define toxicity as a binomial variable (yes vs. no). This binomial variable was used in the antibiotic cohort to standardize the data between the two centers. For the microbiota cohort, only patients treated more recently (2019 and beyond) when both MSK and Penn used the ASTCT consensus grading were included. Therefore, the toxicity grading for the two centers in the fecal microbiome cohort as consistent

### Clinical Data Analysis.

Analysis of both patient samples and healthy control samples was approved under biospecimen research protocols. At MSK, patients were approved under IRB Protocol No. 16-834 for retrospective collection of clinical data. Patients and healthy volunteers provided written informed consent for the prospective collection and use of their fecal samples under IRB Protocol No. 06-107. At Penn, retrospective data collection was performed on IRB Protocol No. UPCC-44420, whereas prospective data collection was approved on IRB Protocol No. UPCC-37418.

### Antibiotic Cohort Analysis.

Patient antibiotic exposures were retrospectively collected from patient records at each institution. Baseline exposure was considered for any antibiotic exposure between Day −30 and the day of CD19 CAR T cell infusion. The duration of antibiotic exposure was not incorporated in the analysis as the period of antibiotic exposure was relatively limited (30 days), and most patients received a full course of antibiotics (5-7 days). Moreover, it is known that even a single dose of an antibiotic can alter the microbiota. Thus, a single dose of an antibiotic was categorized as exposure.

The patients were referred to these Centers to be evaluated to receive CAR T cell treatment several weeks before CAR T infusion in order to undergo verification of eligibility, consent for therapy and apheresis. Exposure was exposed to (a) any antibiotic exposure, (b) piperacillin-tazobactam, imipenem and/or meropenem (P-I-M) and (c) cefepime. Of note, ertapenem was not administered to any patients in this cohort in the 30 days prior to CAR T cell infusion.

Analysis was focused on P-I-M since these antibiotics are anaerobically active and known to induce dysbiosis based on literature and previous studies. Antibiotics more commonly given to these patients (e.g., neutropenic fever) were focused on so that the findings and potential remedies could be applicable to a broad population.

Moreover, other anaerobically active antibiotics were analyzed, including clindamycin, metronidazole, and oral vancomycin. It was found that most patients who received one of these three other anaerobically active antibiotics in the 30 days prior to CAR T cell infusion also received P-I-M. Hence, it could not assess whether exposure to these medications alone had a similar impact on clinical outcomes as exposure to P-I-M given the small sample size.

For the antibiotic data, the assessment of OS and PFS included different covariates depending on analysis: 1. The Cox models were stratified by center and disease when the figure included both diseases (ALL and NHL) and both centers (MSK and Penn). 2. The Cox models were stratified by center when the figure included one disease (ALL or NHL) and both centers (MSK and Penn). 3. The Cox models were stratified by disease when the figure included both diseases (ALL and NHL) and one location (MSK or Penn).

### Center Specific Antibiotic Protocols.

MSK guidelines for antibiotic prophylaxis of CAR T patients included antiviral treatment (acyclovir prophylaxis commencing with chemotherapy and continue for minimum of 6 months post-CAR T infusion.), antifungal prophylaxis (posaconazole or voriconazole 48 hours prior to the start of cyclophosphamide and until neutrophil recovery per clinician decision), antibacterial prophylaxis (levofloxacin considered commencing with chemotherapy and discontinuing upon neutrophil recovery or initiation of broad-spectrum antibiotics for fever and neutropenia for all patients receiving fludarabine/cyclophosphamide and being managed as an outpatient), PJP prophylaxis (administered to all patient receiving lymphodepletion regimen).

Penn guidelines for antibiotic prophylaxis of CAR T patients included antiviral treatment (acyclovir/valaciclovir prophylaxis starting from Day 0 until at least Day 30 post-CAR T cell infusion), antifungal prophylaxis (fluconazole Day 0-30 per clinician decision or routinely to all patients receiving fludarabine/cyclophosphamide lymphodepletion regimen), antibacterial prophylaxis (levofloxacin Day 0-10 to all patients receiving fludarabine/cyclophosphamide and to patients with prolonged neutropenia prior to infusion), PJP prophylaxis was administered to all patient receiving fludarabine/cyclophosphamide lymphodepletion regimen and per clinical decision in all remaining patients.

### Fecal Microbiome Collection and Processing.

The fecal sample collection occurred from 2017 to 2020. Specifically, the range for collection at MSK was 2017 to 2020 and the range for collection at Penn was 2019 to 2020. All patients consented to biospecimen protocols, 06-107 or 09-141 (MSK) and UPCC37418 (Penn). All the patients in the fecal microbiome cohort are also analyzed in the antibiotic cohort.

The samples were collected prospectively at the clinical facilities of MSK or Penn. Upon collection, they were aliquoted and frozen (−80 C) within 24 hours in all but two samples in laboratories at MSK or Penn. We did not utilize a preservative in the processing of the samples.

At both MSK and Penn, some of the fecal samples were collected before conditioning chemotherapy, while some fecal samples were collected after conditioning therapy was administered. Specifically, at MSK, 32% (n=9) of samples were collected before conditioning, whereas 68% (n=19) of samples were collected after conditioning. At Penn, 85% (n=17) of samples were collected before conditioning, and 15% (n=3) were collected after conditioning. Overall, for the two institutions, the median timing of sample collection relative to lymphodepletion was 0 (range, −29, 10).

DNA was extracted centrally (at MSK). In order to mitigate batch effect, fecal samples were processed in batches and sequenced together at the MSK sequencing core given that prior studies have shown that relative, not absolute, measures are comparable between protocols. Three patient samples were excluded from 16S sequencing, whereas four were excluded from metagenomic shotgun sequencing due to inadequate fecal material, lack of amplification, or failure quality control measures (FIG. 6). For the fecal microbiome cohort, the analysis was limited to fecal specimens that were prospectively collected within 30 days prior to CD19 CAR T cell infusion.

16S rRNA Amplicon Sequencing and Bioinformatic Pipeline Analysis.

For the 16S rRNA sequencing, bacterial cell walls were disrupted using silica bead-beating, nucleic acids were isolated using phenol-chloroform extraction, and the V4-V5 variable region of the 16S rRNA gene was amplified with polymerase chain reaction (PCR). The median read count was 50,788 and the range was from 7,041 to 93,953. 16S amplicons were purified either using a Qiagen PCR Purification Kit (Qiagen, USA) or AMPure magnetic beads (Beckman Coulter, USA) and quantified using a Tape station instrument (Agilent, USA). DNA was pooled to equal final concentrations for each sample and then sequenced using the Illumina MiSeq platform as previously described in previous publications. The 16S sequencing data was analyzed using the R package DADA2 (version 1.16.0) pipeline with default parameters except for maxEE=2 and truncQ=2 in filterandtrim( ) function, 16S Fastq files were capped at 100K reads per sample. Amplicon sequence variants (ASVs) were annotated according to NCBI 16S database using BLAST.

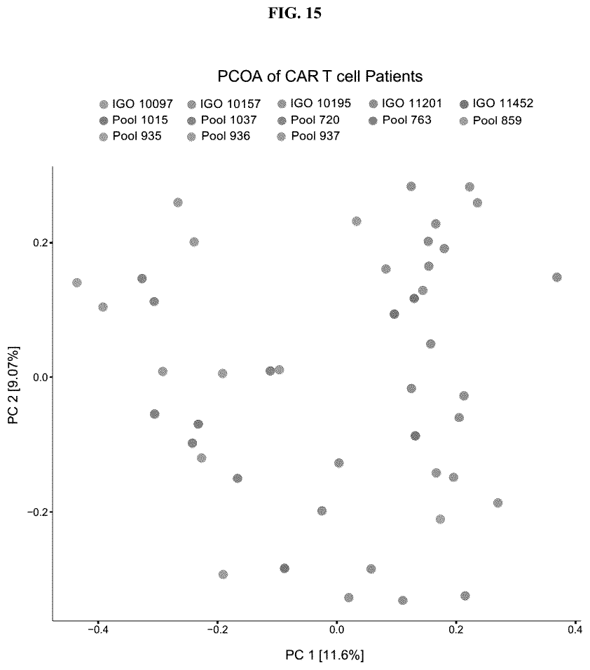

The various pools of 16S sequencing were assessed in a PCoA plot in which the samples were colored by pool ID. In this plot, 45 baseline fecal samples were sequenced in 13 different batches. The samples were well-mixed across the various pools of sequencing (FIG. 15). Hence, batch effect was not a concern in this analysis. Additionally, in the PCoA modeling, the center from which the sample was collected is incorporated as a covariate.

Alpha-diversity was evaluated using the Inverse Simpson index. Beta-diversity matrix was computed using Bray-Curtis dissimilarities at the genus level. Linear discriminant analysis Effect Size (LEfSe) was applied to 16S compositional data. LEfSe identified differentially abundant bacteria between groups with a linear discriminant analysis (LDA) score threshold>4. The abundance threshold for LEfSe was 0.01% and the prevalence threshold was 25%. In this small cohort of 45 patients, the updated analysis will reflect taxa present in at least 12 patients.

### Metagenomic Shotgun Sequencing and Analysis.

For shotgun metagenomic sequencing, DNA was extracted as described above and then sheared to a target size of 650 bp using a Covaris ultrasonicator. DNA was then prepared for sequencing using the Illumina TruSeq DNA library preparation kit and sequenced using the Illumina HiSeq system targeting ˜10-20×106 reads per sample with 100 bp, paired-end reads.

The right and left side of a read in a pair was trimmed to Q10 using the Phred algorithm, using the bbduk.sh script in the BBMap package (BBMap—Bushnell B.—https://www.sourceforge.net/projects/bbmap/). A pair of reads was dropped if any one of them had a length shorter than 51 nucleotides after trimming. The 3′-end adapters were trimmed using a kmer of length 31, and a shorter kmer of 9 at the other end of the read. One mismatch was allowed in this process, and adapter trimming was based on pair overlap detection (which does not require known adapter sequences) using the ‘tbo’ parameter. The ‘tpe’ parameter was used to trim the pair of reads to the same length. For the shotgun data, after preprocessing and decontamination, the median read depth was 19,476,595. The range was from 6,106,173 to 48,289,024.

Removal of human contamination was done using Kneaddata with paired end reads, employing BMTagger. The BMTagger database was built with human genome assembly GRCh38. After decontamination, the paired-end reads were concatenated to a single FASTQ file as the input for functional profiling with the HUMAnN 3.0 ( ) pipeline. After aligning to the updated ChocoPhlAn and UniRef90 database with default settings, the samples were renormalized by library depth to copies per million. MetaCyc was used to obtain stratified and unstratified pathway abundances.

Renormalized pathway abundance tables of the samples were contrasted between the binary outcome of toxicity or complete response at Day 100 using LEfSe. LEfSe was also utilized to assess differential abundance of MetaCyc pathways with an LDA score threshold>2. The abundance threshold for LEfSe was 50 copies per million (0.01%) and the prevalence threshold was 25%. In this small cohort of 45 patients, the updated analysis will reflect taxa present in at least 12 patients.

### Computational Analysis.

Unless otherwise noted, computational analyses were performed using R version 4.1.1. The following R packages were used for the data analysis: parallel, stats, graphics, grDevices, utils, datasets, methods, base, rethinking (version 2.13), rstan (version 2.21.2), StanHeaders (version 2.21.0-7), vegan (version 2.5-7), lattice (version 0.20-44), permute (version 0.9-5), ggpubr (version 0.4.0), vdbR (version 0.0.0.9000), RPostgreSQL (version 0.6-2), DBI (version 1.1.1), Rtsne (version 0.15), ape (version 5.5), labdsv (version 2.0-1), mgcv (version 1.8-36), nlme (version 3.1-152), data.table (version 1.14.0), forcats (version 0.5.1), stringr (version 1.4.0), dplyr (version 1.0.7), purrr (version 0.3.4), readr (version 2.0.1), tidyr (version 1.1.3), tibble (version 3.1.4), ggplot2 (version 3.3.5), and tidyverse (version 1.3.1).

Functional profiling was carried out with HUMAnN v.3.0 (; Methods).

### Statistical Analysis.

Analyses of processed data were conducted using the R software package (version 3.6.1). Two-tailed P values less than 0.05 were considered statistically significant across all tests. Wilcoxon rank-sum tests were used to compare the pairwise association between continuous variables and a binary outcome, while Fisher's exact test was used to compare two categorical variables. OS and PFS survival curves were estimated using Kaplan-Meier curves. A log-rank test compared survival for various antibiotic categories. For comparisons that combined data from Penn and MSK, a stratified test statistic was used. A Cox proportional hazards models was used to adjust for other clinical factors; the model was similarly stratified by institution.

### Bayesian Modeling Analysis.

Using a Bayesian approach, posterior distribution of the coefficients was obtained, which retains the uncertainty of the coefficient estimates. The posterior predictive values were also evaluated to quantify the impact of the coefficients.

The Bayesian modeling was conducted using the rethinking package (version 2.13). The yes response for toxicity and CR Day 100 was converted to 1 and no response to 0, and the Inverse Simpson diversity index was log transformed and then standardized before entering the model. The model was constructed with toxicity or CR response as the outcome with a logit link function and transformed standardized diversity as the predictor while also incorporating random intercepts to adjust for center-wise difference, running on 4 chains using 8 cores. The covariates included in the model are as follows: CR/Toxicity˜alpha diversity+Center.

The prior distribution for the predictor's coefficient is set to be a normal distribution with mean 0 and standard deviation 2. The prior distribution for the random intercepts is set to be a normal distribution with mean 0 and standard deviation 0.5. To visualize and contrast the association between diversity and outcome, a hypothetical higher diversity was considered for one standard deviation above the mean on the log scale, whereas one standard deviation below the mean was deemed lower diversity. The probability distribution for having a toxicity or CR response was calculated from the posterior distribution of the coefficients and contrasted between the higher and lower diversity at the two centers. The aggregated version that combined two centers were shown in the figures.

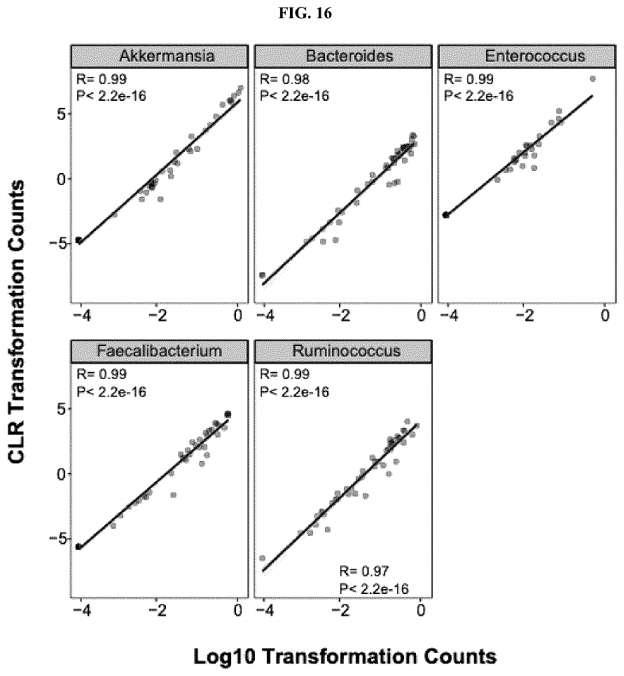

Akkermansia, Bacteroides, Enterococcus, Faecalibacterium, and Ruminococcus were selected based on the immune checkpoint blockade literature to investigate the correlation between genera relative abundance and the response to toxicity as well as Day 100 CR. The genera relative abundance was first log transformed with a pseudocount of 2×10⁶. We visualized the Pearson correlation between the centered log-ratio (CLR) and the log 10 transformed counts for the five genera that was put into the Bayesian model (FIG. 16). It was found that the correlation is about 1 for all the genera. Therefore, it was determined that the log 10 transformation was appropriate to use for this analysis. The model was built with the log 10 transformed relative abundance of the 5 genera along with a random intercept for the centers, running on 4 chains using 8 cores. The covariates included in the model are as follows: CR/Toxicity˜Akkermansia+Bacteroides+Enterococcus+Faecalibacterium+Ruminococcus+Center.

The prior distribution for the genera's coefficient is set to be a normal distribution with mean 0 and standard deviation 1, while that for the random intercepts is set to be a normal distribution with mean 0 and standard deviation 0.5 as well. To understand the implication of the coefficient for Ruminococcus, the patients that had Ruminococcus abundance in the top 10% quantiles and bottom 10% quantiles were identified. The “high” represents the samples that had the top 10% Ruminococcus/Bacteroides relative abundance among all the samples, while “low” pertained to the samples with the bottom 10%. Then, the log relative abundance for the 5 genera was integrated with the 100 random draws from the coefficients' posterior distribution to compute a probability distribution for having a toxicity or CR response. This process was repeated similarly for a situation where patients had top 10% quantiles, and bottom 10% quantiles of Bacteroides abundance observed in this dataset.

### Results

Antibiotic Exposure Before CD19 CAR T Cell Therapy is Associated with Reduced Progression-Free and Overall Survival.

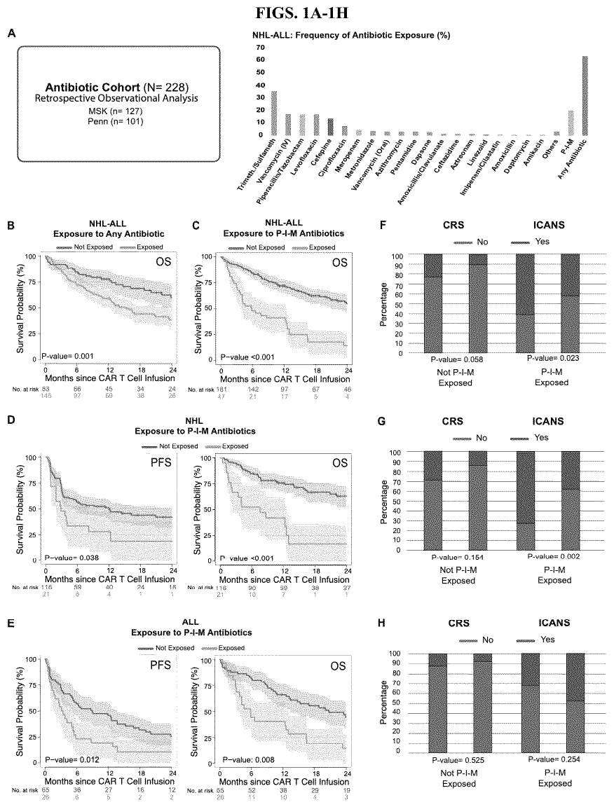

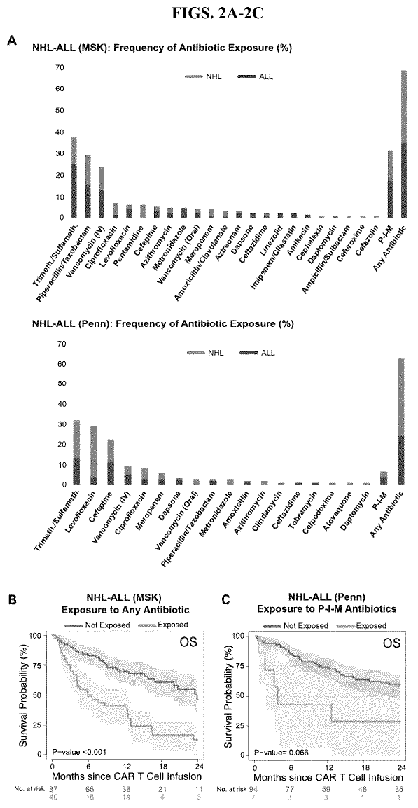

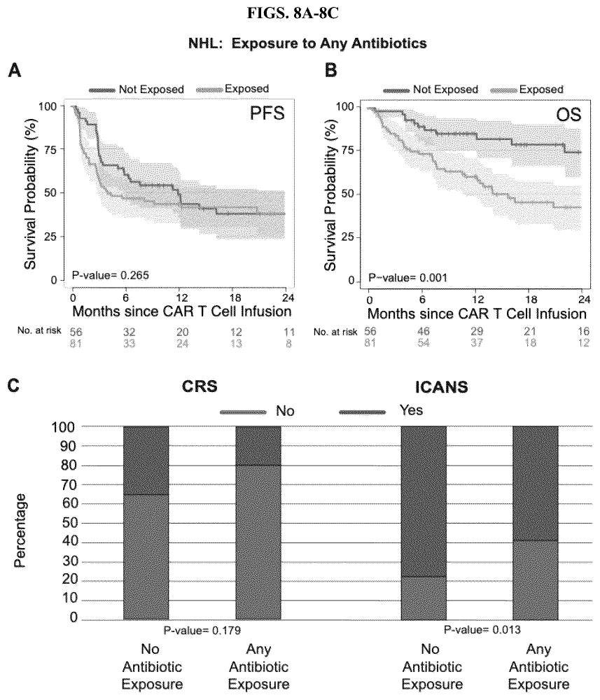

To explore the role of the intestinal microbiome in the response to CD19-targeted CAR T cell therapy, the association between exposure to antibiotics and clinical outcomes were first investigated, since antibiotics are well-known to change the composition of the intestinal microbiota communities. Clinical data on patients treated with investigational or commercial CD19 CAR T cells at two institutions, MSK and Penn, were retrospectively collected. The combined cohort included patients with both acute lymphoblastic leukemia (ALL, n=91) and non-Hodgkin lymphoma (NHL, n=137) (Table 1). Exposure to any kind of antibiotic during the four-week period before CAR T cell infusion was first assessed. Overall, approximately 60% of NHL and ALL patients received at least one antibiotic in the month before CD19 CAR T cell therapy. The most used antibiotics that patients received were trimethoprim-sulfamethoxazole, intravenous vancomycin, piperacillin-tazobactam, levofloxacin, cefepime, ciprofloxacin and meropenem (FIG. 1A). A similar percentage of patients were exposed to antibiotics in both the MSK and Penn cohorts (FIG. 2A). Of note, antibiotic exposure was associated with worse overall survival (OS) (FIG. 1B; OS hazard ratio (HR), 1.71; 95% confidence interval (CI), 1.12-2.59; p=0.011). Likewise, when a more homogenous subgroup of patients with NHL was assessed, an association between any antibiotic exposure and decreased OS was found but not progression-free survival (PFS) (FIG. 8A-8B; PFS HR, 1.29; 95% CI, 0.82-2.01; p=0.265; OS HR, 2.54; 95% CI, 1.41-4.56; p=0.001).

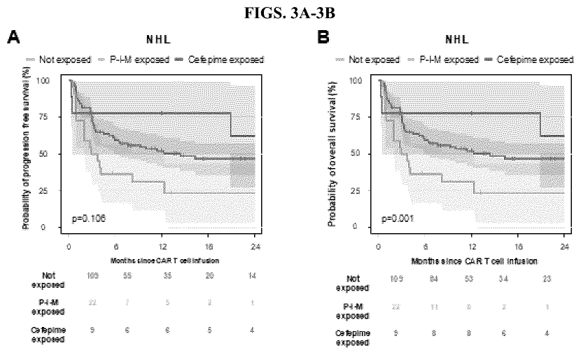

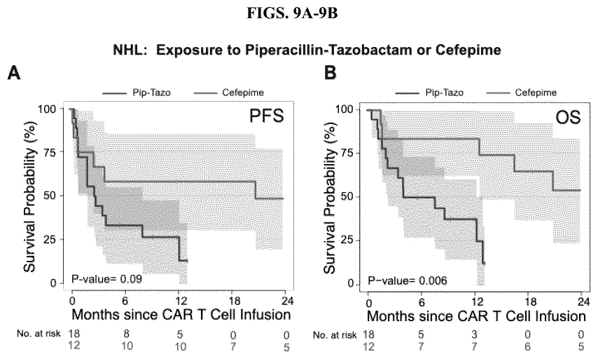

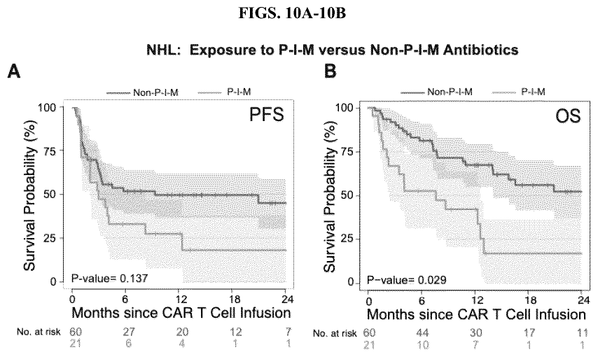

It has been previously observed profound alterations in fecal microbiota communities following exposure to antibiotics, such as piperacillin-tazobactam and imipenem, whose target spectra include anaerobic organisms, in keeping with the observation that many members of the microbial community are strict anaerobes. It was reasoned that treatment with broad spectrum antibiotics that target anaerobes would induce dysbiosis characterized by loss of obligate anaerobes. Thus, this analysis focused on anaerobe-targeting antibiotics used in the setting of neutropenic fever: piperacillin-tazobactam, imipenem-cilastatin and meropenem (here referred to as “P-I-M”) at the exclusion of other anaerobe-targeting medications such as clindamycin, metronidazole, and oral vancomycin. In this regard, only 9 of 228 patients in this cohort were exposed to one of these other anaerobe-targeting antibiotics in the absence of P-I-M. Forty-seven (20.6%) of 228 patients were exposed to P-I-M in the four weeks before CD19-targeted CAR T cell infusion. Patient characteristics at the time of CAR T cell infusion were overall similar between the P-I-M-exposed and not-exposed groups although a worse performance status was observed in patients with NHL patients treated with P-I-M (Table 2). A difference in performance status in patients with ALL treated with P-I-M was not observed (Table 5). It was found that OS was significantly shorter following CAR T cell infusion in patients exposed to P-I-M (FIG. 1C; OS HR, 2.58; 95% CI, 1.68-3.98; p=<0.001). A similar association is observed for OS when analyzed by disease (NHL and ALL), as well as PFS, for the two diseases (FIG. 1D; PFS HR, 1.83; 95% CI, 1.03-3.27; p=0.038; OS HR, 3.37; 95% CI, 1.77-6.44; p=<0.001) (FIG. 1E; PFS HR, 1.96; 95% CI, 1.15-3.35; p=0.012; OS HR, 2.12; 95% CI, 1.2-3.76; p=0.008). When the analysis was separated by institution, it was found that P-I-M antibiotic exposure is associated with worse OS in the MSK cohort. In both diseases, P-I-M antibiotics were used more frequently at MSK (n=40) compared to Penn (n=7) due to institutional guidelines for the management of neutropenic fever (MSK used piperacillin-tazobactam as first-line for neutropenic fever while Penn first used cefepime) (Table 1). However, also in the Penn cohort, P-I-M exposure was associated with a trend towards decreased OS (FIG. 2C); OS HR, 2.37; 95% CI, 0.92-6.09; p=0.066). Since cefepime is a commonly used first-line therapy for neutropenic fever, its effect on CAR T cell immunotherapy outcomes was explored. It was postulated that exposure to cefepime is not associated with decreased survival since it does not target obligate anaerobes to the same degree as P-I-M. Only 9 patients with NHL were treated with cefepime but not exposed to P-I-M. Nevertheless, exposure to cefepime, during the four weeks preceding cell infusion was not associated with worse PFS (FIG. 3A); P-I-M compared to unexposed: PFS HR, 1.81; 95% CI, 1.02-3.23; Cefepime compared to unexposed: PFS HR, 0.68; 95% CI, 0.24-1.93; overall p=0.089) (FIG. 3B; P-I-M: OS HR, 3.13; 95% CI, 1.64-5.96; Cefepime compared to unexposed: OS HR, 0.69; 95% CI, 0.21-2.29; overall p=<0.001). Further, it was found that in patients with NHL exposure to piperacillin-tazobactam as compared to cefepime is associated with worse OS but not worse PFS (FIG. 9A-9B; PFS HR, 0.42; 95% CI, 0.15-1.18; p=0.09; OS HR, 0.18; 95% CI, 0.05-0.68; p=0.006); however, this analysis is limited as receipt of these two antibiotics are highly associated with each center. Moreover, exposure to P-I-M versus non-P-I-M antibiotics was compared to understand their relative role in affecting CAR T outcomes. Interestingly, it was found that P-I-M is associated with worse OS but not worse PFS (10A-10B; PFS HR, 1.65; 95% CI, 0.85-3.21; p=0.137; OS HR, 2.19; 95% CI, 1.07-4.47; p=0.029). Overall, exposure to P-I-M in the four weeks prior to CAR T cell infusion is associated with worse OS and PFS.

TABLE 1
Antibiotic Cohort: Patient Characteristics by Institution.

| | Total | | MSK | | Penn | |
|---|---|---|---|---|---|---|
| Characteristics | N = 228 (100%) | | n = 127 (55.7%) | | n = 101 (44.3%) | |
| Gender | | | | | | |
| Male | 159 | (69.7) | 90 | (70.1) | 69 | (68.3) |
| Female | 69 | (30.3) | 37 | (29.1) | 32 | (31.7) |
| Age - median [IQR] | 56 | [41-66] | 58 | [43-67] | 54 | [37-64] |
| Disease | | | | | | |
| ALL | 91 | (39.9) | 55 | (43.3) | 36 | (35.6) |
| NHL | 137 | (60.1) | 72 | (56.7) | 65 | (64.4) |
| Previous lines of therapy | | | | | | |
| ≤4 | 131 | (57.5) | 75 | (59.1) | 56 | (55.4) |
| >4 | 97 | (42.5) | 52 | (40.9) | 45 | (44.6) |
| Performance status | | | | | | |
| (ECOG) | | | | | | |
| 0-1 | 205 | (89.9) | 107 | (89.2) | 98 | (98.0) |
| ≥2 | 15 | (6.6) | 13 | (10.8) | 2 | (2.0) |
| Missing | 8 | (3.5) | | | | |
| Disease status at infusion | | | | | | |
| Complete response | 26 | (11.4) | 23 | (18.1) | 3 | (3.0) |
| Persistent disease | 202 | (88.6) | 104 | (81.9) | 98 | (97.0) |
| Specific CD19 CAR T cell | | | | | | |
| product | | | | | | |
| 19-28z | 55 | (24.1) | 55 | (43.3) | 0 | (0.0) |
| axicabtagene ciloleucel | 72 | (31.6) | 48 | (37.8) | 24 | (23.8) |
| tisagenlecleucel | 101 | (44.3) | 24 | (18.9) | 77 | (76.2) |
| Costimulatory domain | | | | | | |
| CD28 | 127 | (55.7) | 103 | (81.1) | 24 | (23.8) |
| 4-1BB | 101 | (44.3) | 24 | (18.9) | 77 | (76.2) |
| Toxicity | | | | | | |
| No | 40 | (17.5) | 21 | (16.5) | 19 | (19.2) |
| Yes | 186 | (81.6) | 106 | (83.5) | 80 | (80.8) |
| Missing | 2 | (0.9) | | | | |
| CRS | | | | | | |
| No | 47 | (20.6) | 25 | (19.7) | 22 | (21.8) |
| Yes | 181 | (79.4) | 102 | (80.3) | 79 | (78.2) |
| ICANS/Neurotoxicity | | | | | | |
| No | 118 | (51.8) | 69 | (54.3) | 49 | (61.3) |
| Yes | 89 | (39.0) | 58 | (45.7) | 31 | (38.8) |
| Missing | 21 | (9.2) | | | | |
| Complete response, Day | | | | | | |
| 100 | | | | | | |
| Yes | 117 | (51.3) | 63 | (49.6) | 54 | (53.5) |
| No | 111 | (48.7) | 64 | (50.4) | 47 | (46.5) |
| Vital status | | | | | | |
| Alive | 126 | (55.3) | 65 | (51.2) | 61 | (60.4) |
| Dead | 102 | (44.7) | 62 | (48.8) | 40 | (39.6) |
| Antibiotic exposure | | | | | | |
| No | 83 | (36.4) | 40 | (31.5) | 43 | (42.6) |
| Yes | 145 | (63.6) | 87 | (68.5) | 58 | (57.4) |
| P-I-M antibiotic exposure | | | | | | |
| No | 181 | (79.4) | 87 | (68.5) | 94 | (93.1) |
| Yes | 47 | (20.6) | 40 | (31.5) | 7 | (6.9) |

Patients who were treated with anti-CD19 CAR T cell immunotherapy at two institutions, MSK and Penn, are included in this cohort (N=228). Patients were studied for antibiotic use in the 30 days before CAR T cell infusion and evaluated for clinical characteristics and outcomes. Complete response (CR) denotes whether the patient was in CR when assessed at approximately Day 100 (as opposed to best response of CR by Day 100). Regarding the CAR costimulatory domain, all recipients of a 4-1BB product received tisagenlecleucel (n=72). Recipients of a CD28 product either received an MSK investigational product (n=55) or axicabtagene ciloleucel (n=101). Toxicity is defined as either cytokine release syndrome (CRS) of any grade or immune effector cell-associated neurotoxicity syndrome (ICANS) or neurotoxicity of any grade. Vital status is noted within 24 months of follow-up after CAR T cell infusion. Antibiotic exposure denotes exposure to any antibiotic. Abbreviations: IQR: inter-quartile range; ALL: acute lymphoblastic leukemia; NHL: non-Hodgkin lymphoma; ECOG: Eastern Cooperative Oncology Group; P-I-M: piperacillin-tazobactam, imipenem-cilastatin or meropenem; No P-I-M antibiotic exposure: patients exposed to non-P-I-M plus patients who did not receive any antibiotics within 4 weeks before CD19 CAR T cell infusion.

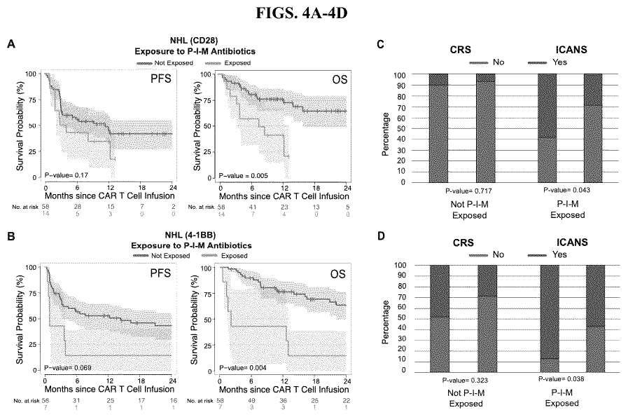

In order to understand whether exposure to P-I-M antibiotics affected CAR T outcomes independently of the CAR costimulatory domain, the survival of NHL was analyzed: patients treated with CD28- versus 4-1BB-costimulated CAR T cells. The analysis was focused on NHL patients because they included patients treated with both costimulatory domains in both institutions, while ALL patients more frequently received CD28-costimulation at MSK and 4-1BB at Penn. In both CD28 and 4-1BB CAR T cell cohorts, OS was lower in P-I-M treated patients (FIG. 4A-4B; CD28: OS HR, 3.68; 95% CI, 1.4-9.67; p=0.005; 4-1BB: OS HR, 3.58; 95% CI, 1.42-9.02; p=0.004), with a trend toward decreased rate of PFS in recipients of CD28 CAR T and a significant decrease in the 4-1BB CAR T cohort (FIG. 4A-4B; CD28: PFS HR, 1.71; 95% CI, 0.79-3.73; p=0.17; 4-1BB: PFS HR, 2.24; 95% CI, 0.92-5.46; p=0.069). Hence, exposure to P-I-M in the four weeks prior to CAR T cell infusion is associated with decreased OS but not PFS in NHL patients irrespective of the CAR costimulatory domain.

It was queried whether patients that were exposed to P-I-M antibiotics were the ones with a more aggressive disease and disease-related complications that led to antibiotic treatment. To evaluate these potential confounders, the relationship between P-I-M antibiotic exposure and overall survival was analyzed in uni- and multi-variable models that included age, gender, disease type, performance status, CAR co stimulatory domain and LDH as variables. Importantly, while the predictive role of ECOG performance status and LDH was confirmed, exposure to P-I-M antibiotics remained a strong predictor of shorter OS (HR, 2.54; 95% CI, 1.62-3.97; p=<0.001) status (Table 3).

TABLE 2
Antibiotic Cohort: Non-Hodgkin Lymphoma Patient Characteristics by Exposure
to Piperacillin-Tazobactam, Imipenem-Cilastatin or Meropenem (P-I-M).
Non-Hodgkin Lymphoma

| | | | No P-I-M | | P-I-M | | |
|---|---|---|---|---|---|---|---|
| | | | antibiotic | | antibiotic | | |
| | Total | | exposure | | exposure | | |
| | n = 137 | | n = 116 | | n = 21 | | |
| Characteristics | (100%) | | (84.7% ) | | (15.3%) | | p-value |
| Gender | | | | | | | |
| Male | 93 | (67.9) | 79 | (68.1) | 14 | (66.7) | 0.897 |
| Female | 44 | (32.1) | 37 | (31.9) | 7 | (33.3) | |
| Age - median [IQR] | 61 | [51-69] | 60 | [52-68] | 63 | [46-70] | 0.843 |
| Center | | | | | | | |
| MSK | 72 | (52.6) | 54 | (46.6) | 18 | (85.7) | 0.001 |
| Penn | 65 | (47.4) | 62 | (53.4) | 3 | (14.3) | |
| Performance status (ECOG) | | | | | | | |
| 0-1 | 120 | (87.6) | 107 | (93.9) | 13 | (72.2) | 0.003 |
| ≥2 | 12 | (8.8) | 7 | (6.1) | 5 | (27.8) | |
| Missing | 5 | (3.6) | | | | | |
| Previous lines of therapy | | | | | | | |
| ≤4 | 77 | (56.2) | 69 | (59.5) | 8 | (38.1) | 0.069 |
| >4 | 60 | (43.8) | 47 | (40.5) | 13 | (61.9) | |
| Disease status at infusion | | | | | | | |
| Complete response | 4 | (2.9) | 4 | (3.4) | 0 | (0.0) | 0.388 |
| Persistent disease | 133 | (97.1) | 112 | (96.6) | 21 | (100.0) | |
| Costimulatory domain | | | | | | | |
| CD28 | 72 | (52.6) | 58 | (50.0) | 14 | (66.7) | 0.159 |
| 4-1BB | 65 | (47.4) | 58 | (50.0) | 7 | (33.3) | |
| Toxicity | | | | | | | |
| No | 33 | (24.1) | 32 | (27.8) | 1 | (4.8) | 0.023 |
| Yes | 103 | (75.2) | 83 | (72.2) | 20 | (95.2) | |
| Missing | 1 | (0.7) | | | | | |
| CRS | | | | | | | |
| No | 37 | (27.0) | 34 | (29.3) | 3 | (14.3) | 0.154 |
| Yes | 100 | (73.0) | 82 | (70.7) | 18 | (85.7) | |
| ICANS/Neurotoxicity | | | | | | | |
| No | 91 | (66.4) | 83 | (72.8) | 8 | (38.1) | 0.002 |
| Yes | 44 | (32.1) | 31 | (27.2) | 13 | (61.9) | |
| Missing | 2 | (1.5) | | | | | |
| Complete response, Day 100 | | | | | | | |
| Yes | 62 | (45.3) | 55 | (47.4) | 7 | (33.3) | 0.340 |
| No | 75 | (54.7) | 61 | (52.6) | 14 | (66.7) | |
| Vital status | | | | | | | |
| Alive | 88 | (64.2) | 82 | (70.7) | 6 | (28.6) | |
| Dead | 49 | (35.8) | 34 | (29.3) | 15 | (71.4) | |

Non-Hodgkin lymphoma patients from Memorial Sloan Kettering Cancer Center (MSK) and the University of Pennsylvania (Penn) who received anti-CD19 CAR T immunotherapy (N=140) were assessed based upon exposure to piperacillin-tazobactam, imipenem-cilastatin or meropenem (P-I-M) antibiotics in the 30 days before CAR T infusion and evaluated for clinical characteristics and outcomes. The baseline and clinical characteristics of the patients in each of these group was compared to assess for variables that were associated with exposure to these antibiotics. Vital status is noted within 24 months of follow-up after CAR T cell infusion. Abbreviations: IQR: inter-quartile range; NHL=non-Hodgkin lymphoma; ECOG: Eastern Cooperative Oncology Group; CRS: cytokine release syndrome; No P-I-M antibiotic exposure: patients exposed to non-P-I-M plus patients who did not receive any antibiotics within the 4 weeks before CD19 CAR T cell infusion; ICANS: immune effector cell-associated neurotoxicity syndrome.

TABLE 3
Antibiotic Cohort: Uni- and Multivariable Analysis of the Association
of Patient Characteristics and Overall Survival Following CD19-Targeted
CAR T Cell Therapy, Stratified by Institution.
Overall Survival

| | | Univariable* | | | Multivariable* | | |
|---|---|---|---|---|---|---|---|
| Variable | | HR (95% CI) | | P-value | HR (95% CI) | | p-value |
| Gender | Female | (reference) | | 0.157 | | | |
| | Male | 1.37 | (0.89-2.11) | | | | |
| Age | (Continuous) | 1 | (0.99-1.01) | 0.669 | | | |
| Disease | ALL | (reference) | | 0.097 | (reference) | | 0.130 |
| | NHL | 0.72 | (0.48-1.06) | | 0.72 | (0.46-1.1) | |
| Performance | 0 | (reference) | | 0.03 | (reference) | | 0.033 |
| status (ECOG) | 1 | 1.49 | (0.98-2.27) | | 1.59 | (1.0-2.54) | |
| | 2-3 | 2.49 | (1.21-5.15) | | 2.56 | (1.2-5.45) | |
| Previous lines | ≤4 | (reference) | | 0.404 | | | |
| of therapy | >4 | 0.85 | (0.59-1.24) | | | | |
| Costimulatory | 4-1BB | (reference) | | 0.865 | | | |
| domain | CD28 | 1.04 | (0.63-1.73) | | | | |
| LDH | (Continuous, | 1.33 | (1.18-1.5) | <0.001 | 1.34 | (1.18-1.52) | <0.001 |
| | per 100) | | | | | | |
| P-I-M | No | (reference) | | <0.001 | (reference) | | <0.001 |
| antibiotic | Yes | 2.71 | (1.76-4.16) | | 2.58 | (1.55-4.3) | |

exposure

Uni- and multivariable Cox proportional-hazards analyses of the association of decreased overall survival in patients who were exposed to piperacillin-tazobactam, imipenem-cilastatin or meropenem (P-I-M) in the 30 days prior to CD19-targeted CAR T cell therapy. LDH indicates a measurement prior to lymphodepletion. The statical threshold for inclusion of a variable in the multivariate model is <0.10. *Univariable and multivariable Cox proportional hazards models were stratified based on institution. Abbreviations: IQR: inter-quartile range; ALL: acute lymphoblastic leukemia; NHL: non-Hodgkin lymphoma; ECOG: Eastern Cooperative Oncology Group; LDH: lactate dehydrogenase.
P-I-M Exposure Before CD19 CAR T Cell Therapy is Associated with Increased ICANS.

It was then investigated whether exposure to any antibiotic is associated with CAR-mediated toxicities, such as CRS and ICANS. It was found that exposure to any antibiotic in the 30 days prior to CAR T cell infusion is associated with increased ICANS in patients with NHL (p=0.013) (FIG. 8C). Next, the association between P-I-M and CAR-mediated toxicities was assessed. Exposure to P-I-M is associated with increased ICANS (p=0.023) but not CRS (p=0.058) in patients in the combined NHL and ALL cohort, as well as in patients with NHL (CRS: p=0.154, ICANS: p=0.002) (FIG. 1F, 1G, 1H). However, exposure to P-I-M is not significantly associated with CRS or ICANS in patients with ALL (CRS: p=0.525, ICANS: p=0.254) (FIG. 1H). Finally, it was observed that P-I-M exposure in the NHL patients was associated with a higher rate of ICANS events irrespective of the costimulatory domain used (CD28: p=0.038, 4-1BB: p=0.038) (FIG. 4C, 4D). These findings are consistent with a potential association of the intestinal microbiota with ICANS through the gut-brain axis that plays a substantial role in the regulation of CNS autoimmunity, inflammation, and immune cell trafficking.

In conclusion, it was found that exposure to piperacillin/tazobactam, meropenem, or imipenem in the four weeks before CD19 CAR T cell is associated with worse survival (OS and PFS) and increased toxicity (ICANS) in patients with ALL and NHL. These findings held up in a subset analysis of patients stratified by institution (NHL-ALL) (FIG. 2) or recipients of CAR T cells with a 4-1BB costimulatory domain (NHL) (FIG. 4).

### The Microbiome of CD19 CAR T Cell Recipients is Altered Before Therapy.

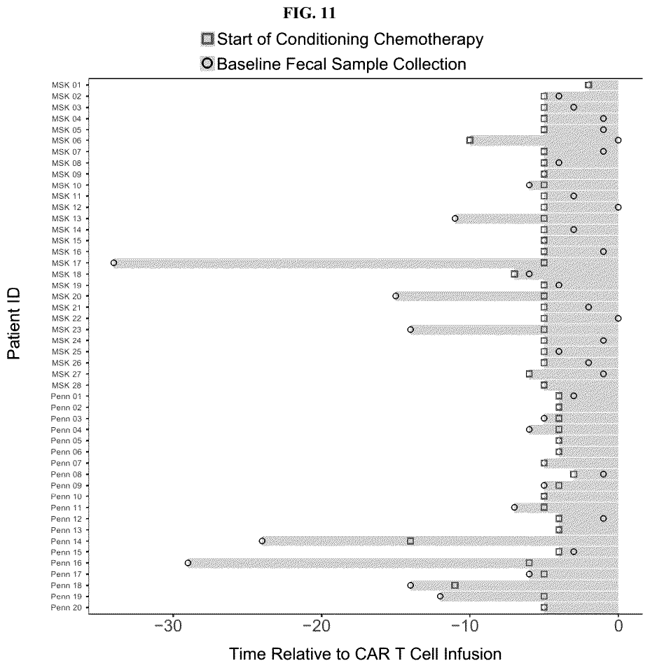

To characterize the fecal microbiome of CAR T cell recipients, collected baseline stool samples from patients at MSK and Penn (n=48) to evaluate the association between the composition of the fecal microbiome and outcomes of anti-CD19 CAR T cell therapy. The baseline fecal samples were all collected prior to CAR T cell infusion but not necessarily prior to the onset of conditioning chemotherapy (FIG. 5A, FIG. 11). Of the forty-eight fecal samples, 14 (29%) were collected before the start of conditioning chemotherapy (FIG. 11). In the fecal microbiome cohort, there is no significant difference in patient characteristics at the time of CAR T cell infusion between patients treated with P-I-M antibiotics compared to untreated patients. (Table 4).

TABLE 4
Fecal Microbiome Cohort: Patient Characteristics.

| | | | | No P-I-M | | P-I-M | | |
|---|---|---|---|---|---|---|---|---|
| | | | | antibiotic | | antibiotic | | |
| | | Total | | exposure | | exposure | | |
| | | N = 48 | | n = 43 | | n = 5 | | |
| | Category | (100%) | | (89.6%) | | (10.4%) | | p-value |
| Institution | MSK | 28 | (58.3) | 23 | (53.5) | 5 | (100.0) | 0.129 |
| | Penn | 20 | (41.7) | 20 | (46.5) | 0 | (0.0) | |
| Age, median [IQR] | | 64 | [55-70] | 64 | [55-70] | 70 | [56-70] | 0.64 |
| Gender | Female | 14 | (29.2) | 13 | (30.2) | 1 | (20.0) | >0.999 |
| | Male | 34 | (70.8) | 30 | (69.8) | 4 | (80.0) | |
| Disease | ALL | 2 | (4.2) | 2 | (4.7) | 0 | (0.0) | >0.999 |
| | NHL | 46 | (95.8) | 41 | (95.3) | 5 | (100.0) | |
| Prior Lines of Therapies - | | 4 | [3-5] | 4 | [3-5] | 6 | [5-6] | 0.111 |
| median [IQR] | | | | | | | | |
| Specific CD19 CAR | 1928z | 2 | (4.2) | 2 | (4.7) | 0 | (0.0) | 0.384 |
| T cell product | axicabtagene | 21 | (43.8) | 17 | (39.5) | 4 | (80) | |
| | ciloleucel | | | | | | | |
| | tisagenlecleucel | 23 | (47.9) | 22 | (51.2) | 1 | (20.0) | |
| | brexucabtagene | 2 | (4.2) | 2 | (4.7 | 0 | (0.0) | |
| | autoleucel | | | | | | | |
| Costimulatory | 4-1BB | 23 | (47.9) | 22 | (51.2) | 1 | (20.0) | 0.397 |
| Domain | CD28 | 25 | (52.1) | 21 | (48.8) | 4 | (80.0) | |
| Complete | Yes | 23 | (47.9) | 22 | (51.2) | 1 | (20.0) | 0.397 |
| response, Day 100 | No | 25 | (52.1) | 21 | (48.8) | 4 | (80.0) | |
| Toxicity | Yes | 33 | (68.8) | 28 | (65.1) | 5 | (100.0) | 0.279 |
| | No | 15 | (31.2) | 15 | (34.9) | 0 | (0.0) | |
| Cytokine release | Yes | 30 | (62.5) | 26 | (60.5) | 4 | (80.0) | 0.714 |
| syndrome | No | 18 | (37.5) | 17 | (39.5) | 1 | (20.0) | |
| CRS Grade | 0 | 18 | (37.5) | 17 | (39.5) | 1 | (20.0) | 0.817 |
| | 1 | 15 | (31.2) | 13 | (30.2) | 2 | (40.0) | |
| | 2 | 14 | (29.2) | 12 | (27.9) | 2 | (40.0) | |
| | 3 | 1 | (2.1) | 1 | (2.3) | 0 | (0.0) | |
| ICANS/ | Yes | 12 | (25.0) | 8 | (18.6) | 4 | (80.0) | 0.014 |
| Neurotoxicity | No | 36 | (75.0) | 35 | (81.4) | 1 | (20.0) | |
| ICANS Grade | 0 | 36 | (75.0) | 35 | (81.4) | 1 | (20.0) | <0.001 |
| | 1 | 6 | (12.5) | 5 | (11.6) | 1 | (20.0) | |
| | 2 | 3 | (6.2) | 0 | (0.0) | 3 | (60.0) | |
| | 3 | 3 | (6.2) | 3 | (7.0) | 0 | (0.0) | |
| Vital Status, last | Alive | 33 | (68.8) | 32 | (74.4) | 1 | (20.0) | |
| follow-up | Dead | 15 | (31.2) | 11 | (25.6) | 4 | (80.0) | |

Fecal microbiome samples from patients at MSK and Penn who received anti-CD19 CAR T cell immunotherapy (N=48) were prospectively collected. Patient characteristics are listed based on exposure to piperacillin-tazobactam, imipenem-cilastatin or meropenem (P-I-M) in the 30 days before CAR T cell infusion and evaluated for clinical characteristics and outcomes. Regarding the CAR costimulatory domain, all recipients of a 4-1BB product received tisagenlecleucel (n=23). Recipients of a CD28 product either received an MSK investigational product (n=2), axicabtagene ciloleucel (n=21), or brexucabtagene autoleucel (n=2). Abbreviations: CR: complete response; ALL: acute lymphoblastic leukemia; NHL: non-Hodgkin lymphoma; CRS: cytokine release syndrome; ICANS: immune effector cell-associated neurotoxicity syndrome; No P-I-M antibiotic exposure: patients exposed to non-P-I-M plus patients who did not receive any antibiotics within the 4 weeks before CD19 CAR T cell infusion.

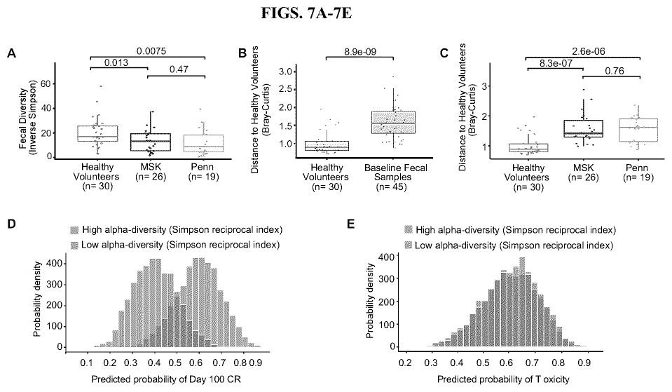

Taxonomic profiling was first performed with 16S ribosomal RNA (rRNA) gene sequencing (n=45) and metagenomic shotgun sequencing (n=45) on the baseline stool samples (FIG. 5A, FIG. 6). The results were correlated with clinical outcomes defined as response (Day 100 complete response (CR) and no CR) and toxicity (CRS and ICANS). It was found that the baseline fecal samples had heterogeneous bacterial compositions—including high abundances of commensal Clostridia (pink, orange, and brown) and Bacteroidetes (teal) as well as occasional samples with high abundances of facultative anaerobes that are potential pathobionts, including Escherichia (dark red), Klebsiella (bright red), and Enterococcus (dark green) (FIG. 5B). Next, the Inverse Simpson index was computed per sample, an alpha-diversity metric that considers the number of unique organisms in a sample and the evenness with which they are distributed. Alpha-diversity is a convenient summary metric of an ecological community and has been linked to various diseases. It was found that alpha-diversity in fecal samples of CAR T cell recipients was significantly lower than those of healthy volunteers (n=30) (p=0.0023 when patients from both Institutions were combined; p=0.013 for MSK and p=0.0075 for Penn) (FIG. 5C and FIG. 7A). The healthy volunteers were not queried as to whether they had recently taken antibiotics or whether they had any GI disease. They are classified as healthy controls based on not currently receiving CD19 CAR T therapy. These data suggest that multiple factors, including prior lines of treatment, disease, antibiotic exposure, and dietary intake, shape the baseline microbiome of cancer patients receiving CD19 CAR T therapy. In addition to a decrease in alpha-diversity, community dominance, defined as a relative abundance of any single taxon of greater than 30%, has also been associated with injury to the microbiome. Dominance in 15 of the 45 fecal samples was found from the 16S sequencing. Several taxa were observed as the dominating organism, but dominance was most frequently observed by the genus Akkermansia (6 of the 15 patients) (FIG. 5B).

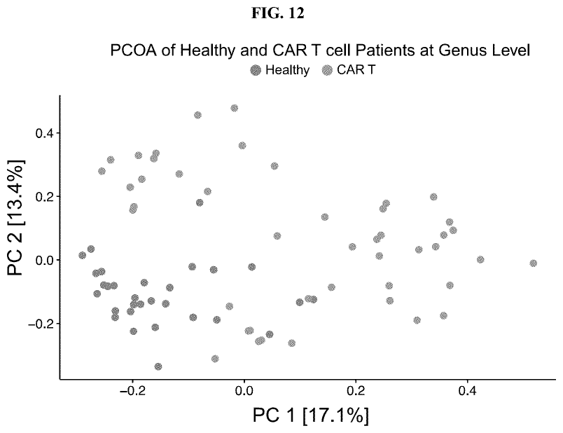

Having observed that patients receive CAR T therapy with abnormally low alpha-diversity, it was sought to characterize the extent to which their global microbiome composition differed from healthy volunteers by measuring Bray-Curtis beta-diversity dissimilarity between each patient sample and a reference point defined by the average of healthy volunteers. It was found that the composition of fecal samples and healthy volunteers was significantly different (p=<0.001 combining patients from both institutions; p=<0.001 (MSK) and p=<0.001 (Penn)) (FIG. 5D and FIG. 12). Principal Coordinates Analysis (PCoA) of the beta-diversity of fecal samples at the amplicon sequence variant (ASV) and genus level demonstrated that the microbiome of CAR T cell patients differed from healthy controls, and both visualizations showed similar levels of separation (FIG. 5D; FIG. 12). It was also found that the composition of fecal samples from CAR T cell recipients was significantly different from healthy volunteers (p=<0.001 (combining patients from both institutions); p=<0.001 (MSK) and p=<0.001 (Penn), FIG. 7B-C). These data suggest that the composition of the baseline microbiome by 16S sequencing of ALL and NHL patients receiving CD19 CAR T therapy is altered compared to healthy individuals. Multiple factors can cause this dysbiosis, including prior lines of treatment, disease, antibiotic exposure, and dietary intake. Hence, these data indicate that CD19 CAR T cell patients have an altered fecal microbiome before cell infusion as measured by lower alpha-diversity, increased frequency of bacterial dominance, and a composition that is distinct from that of healthy volunteers.

The Composition of the Baseline Fecal Microbiome of CAR T Patients Correlates with Clinical Response.

Several studies have found an association between the composition of the intestinal microbiome and the response to cancer therapy, including chemotherapy, immune checkpoint blockade, and allogeneic hematopoietic cell transplantation. Hence, whether differences in the fecal microbiome at baseline are associated with clinical outcomes following treatment with CD19 CAR T cell therapy was investigated. The relationship between alpha-diversity and the odds of Day 100 CR using a Bayesian logistic regression (FIG. 5E upper panel); log-odds ratio, 0.41 [−0.21, 1.05]; mean and 95% highest posterior density intervals [HDI95], respectively). To aid in model interpretation, the posterior probability distribution of Day 100 CR was estimated for a hypothetical patient with a diversity value one standard deviation above the mean, as compared with one with a diversity value one standard deviation below the mean (FIG. 7C). For this hypothetical patient with diversity one standard deviation above the mean on the log scale, the estimated probability of having a Day 100 CR was 20% higher than a patient with diversity one standard deviation below the mean on the log scale (FIG. 7D). An association was not found between alpha-diversity and toxicity analyzed as a binary variable (as defined as Yes or No CRS or ICANS) (FIG. 5E lower panel; log-odds ratio, 0.02 [HDI95: −0.63, 0.58]; posterior distribution, FIG. 7E).

After assessing the association of alpha-diversity and clinical outcomes, whether compositional differences in the fecal microbiome are associated with clinical outcomes following treatment with CD19 CAR T cell therapy were analyzed. High-dimensional class comparisons using linear discriminant analysis of effect size (LEfSe) were performed to characterize taxa associated with clinical response.

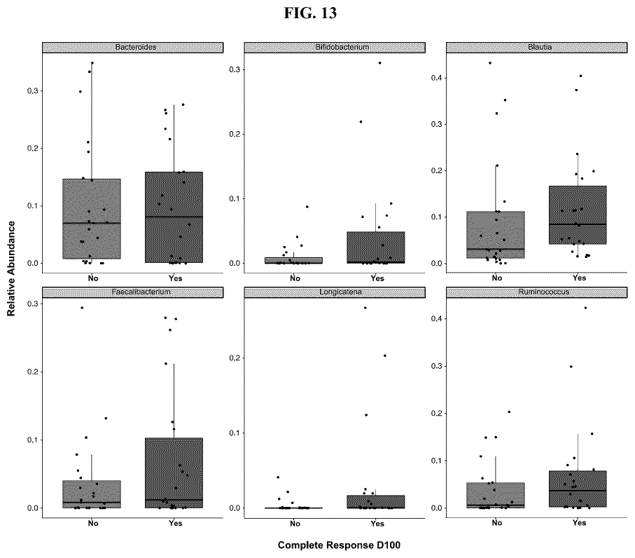

A higher relative abundance of microbial taxa within the class Clostridia, including the genera Ruminococcus and Faecalibacterium, family Ruminococcaceae, as well as the species Faecalibacterium prausnitzii and Ruminococcus bromii was found to be associated with Day 100 CR (FIG. 5F). Notably, all these taxa are obligate anaerobes. It was also noted a higher abundance of the phylum Bacteroidetes and its associated lower classifications: class Bacteroidia, order Bacteroidiales, family Bacteroidaceae, and genus Bacteroides in responders. Conversely, a higher abundance of the order Veillonellales and the family Veillonellaceae are associated with decreased CR at Day 100 (FIG. 5F). The relative abundance of selected taxa and their association with Day 100 CR was analyzed, including Ruminococcus, Bacteroides, and Faecalibacterium (FIG. 13). Overall, it was found that Ruminococcus, Bacteroides, and Faecalibacterium were associated with response to CD19 CAR T cell therapy.

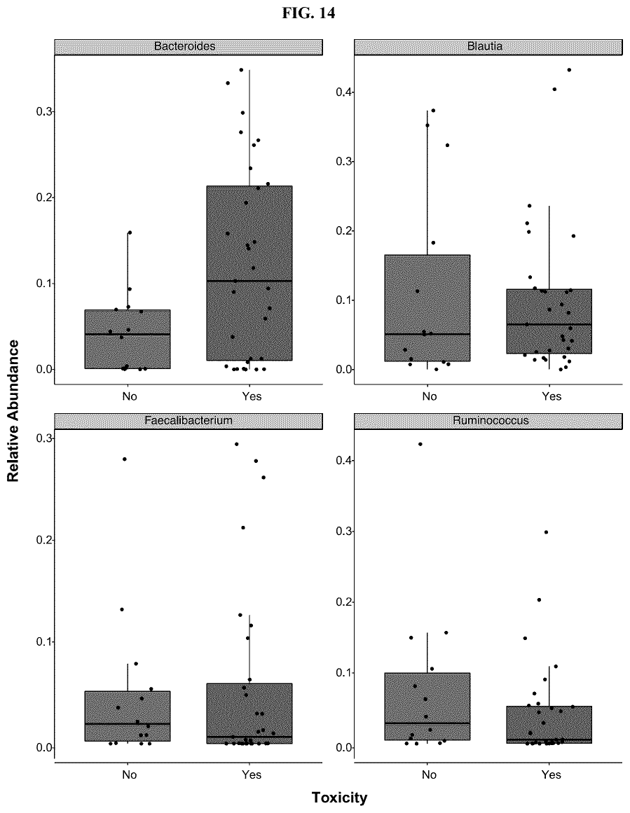

LEfSe was also used to identify taxa that are differentially associated with CAR-mediated toxicity, CRS and ICANS (FIG. 5G). Several abundant microbial taxa were identified in the patients who did not experience toxicity. Interestingly, there is notable overlap between several of the taxa associated with Day 100 CR and no toxicity. In this analysis, it was found that higher abundance of microbial taxa within the class Clostridia, including the genera Ruminococcus and Faecalibacterium, as well as the species Faecalibacterium prausnitzii, was associated with no toxicity. The box plots show the relative abundance of selected taxa of interest for toxicity (FIG. 14). In sum, the taxa highlighted in the LEfSe plots include important genera, such as Blautia, Ruminococcus, Bacteroides, and Faecalibacterium, that were associated with no toxicity.

Ruminococcus, Bacteroides, and Faecalibacterium were associated with Day 100 CR in the untargeted LEfSe analysis, and Akkermansia is the top enriched dominant taxa in these patients. These same genera have also been reported to affect cancer immunotherapy. These four bacterial taxa and Enterococcus have been associated with improved response to immune checkpoint blockade therapy, decreased toxicity to immune checkpoint blockade, as well as immune cell dynamics following allogeneic hematopoietic cell transplantation. Based upon their relevance in the literature, these genera were studied further. A hypothesis-driven exploratory analysis was performed to assess the relevance of bacterial taxa that have been associated with improved response to checkpoint blockade therapy, decreased toxicity to checkpoint blockade, and increased lymphocytes following allogeneic hematopoietic cell transplantation. The association of five genera Akkermansia, Bacteroides, Enterococcus, Faecalibacterium, and Ruminococcus with Day 100 CR and toxicity was assessed in a Bayesian logistic regression. A higher abundance of the genus Ruminococcus was found to be associated with increased odds of Day 100 CR (FIG. 5H); log-odds ratio, 0.56 [HDI95: −0.01, 1.19]). The posterior coefficient distributions can be interpreted as on average there is a 35% higher probability of Day 100 CR in a hypothetical patient with Ruminococcus abundance in the top 10% quantile (high) of all investigated samples compositions compared to one with Ruminococcus abundance in the lower 10% quantile (low) (FIG. 5I). Conversely, a Bayesian logistic regression for toxicity did not show an association with any of the genera, including Bacteroides (FIG. 5J); log-odds ratio, 0.28 [HDI95: −0.29, 0.84]). However, as an exploratory visualization of the posterior distribution of the predicted probability for toxicity, a hypothetical patient with Bacteroides abundance in the top 10% quantile (high) of all investigated sample compositions is estimated to have a high probability of toxicity (posterior probability amassed on the right side of 0.5), whereas a hypothetical patient with Bacteroides abundance in the lower 10% quantile (low) may or may not experience toxicity with equal likelihood (FIG. 5K).

TABLE 5
Antibiotic Cohort: Acute Lymphoblastic Leukemia
Patient Characteristics by Exposure to Piperacillin-
Tazobactam, Imipenem-Cilastatin or Meropenem (P-I-M).
Acute Lymphoblastic Leukemia

| | | | No P-I-M | | P-I-M | | |
|---|---|---|---|---|---|---|---|
| | | | antibiotic | | antibiotic | | |
| | Total | | exposure | | exposure | | |
| | N = 91 | | N = 65 | | N = 26 | | p- |
| Characteristics | (100%) | | (71.4%) | | (28.6%) | | value |
| Gender | | | | | | | |
| Male | 66 | (72.5) | 49 | (75.4) | 17 | (65.4) | 0.334 |
| Female | 25 | (27.5) | 16 | (24.6) | 9 | (34.6) | |
| Age - median [IQR] | 43 | [28-59] | 44 | [30-60] | 40 | [28-58] | 0.849 |
| Center | | | | | | | |
| MSK | 55 | (60.4) | 33 | (50.8) | 22 | (84.6) | 0.004 |
| Penn | 36 | (39.6) | 32 | (49.2) | 4 | (15.4) | |
| Performance status | | | | | | | |
| (ECOG) | | | | | | | |
| 0-1 | 85 | (93.4) | 62 | (96.9) | 23 | (95.8) | 0.863 |
| ≥2 | 3 | (3.3) | 2 | (3.1) | 1 | (4.2) | |
| Missing | 3 | (3.3) | | | | | |
| Previous lines of | | | | | | | |
| therapy | | | | | | | |
| ≤4 | 54 | (59.3) | 41 | (63.1) | 13 | (50.0) | 0.251 |
| >4 | 37 | (40.7) | 24 | (36.9) | 13 | (50.0) | |
| Disease status at | | | | | | | |
| infusion | | | | | | | |
| Complete response | 22 | (24.2) | 16 | (24.6) | 6 | (23.1) | 0.877 |
| Persistent disease | 69 | (75.8) | 49 | (75.4) | 20 | (76.9) | |
| Costimulatory domain | | | | | | | |
| CD28 | 55 | (60.4) | 33 | (50.8) | 22 | (84.6) | 0.003 |
| 4-1BB | 36 | (39.6) | 32 | (49.2) | 4 | (15.4) | |
| Toxicity | | | | | | | |
| No | 7 | (7.7) | 5 | (7.8) | 2 | (7.7) | 0.985 |
| Yes | 83 | (91.2) | 59 | (92.2) | 24 | (92.3) | |
| Missing | 1 | (1.1) | | | | | |
| CRS | | | | | | | |
| No | 10 | (11.0) | 8 | (12.3) | 2 | (7.7) | 0.525 |
| Yes | 81 | (89.0) | 57 | (87.7) | 24 | (92.3) | |
| Neurotoxicity | | | | | | | |
| No | 27 | (29.7) | 15 | (32.6) | 12 | (46.2) | 0.254 |
| Yes | 45 | (49.4) | 31 | (67.4) | 14 | (53.8) | |
| Missing | 19 | (20.9) | | | | | |
| Complete response, | | | | | | | |
| Day 100 | | | | | | | |
| Yes | 55 | (60.4) | 44 | (67.7) | 11 | (42.3) | 0.046 |
| No | 36 | (39.6) | 21 | (32.3) | 15 | (57.7) | |
| Vital status | | | | | | | |
| Alive | 38 | (41.8) | 33 | (50.8) | 5 | (19.2) | |
| Dead | 53 | (58.2) | 32 | (49.2) | 21 | (80.8) | |

Acute lymphoblastic leukemia (ALL) patients from Memorial Sloan Kettering Cancer Center (MSK) and the University of Pennsylvania (Penn) who received anti-CD19 CAR T cell immunotherapy (N=91) were assessed based upon exposure to piperacillin-tazobactam, imipenem-cilastatin, or meropenem (P-J-M) antibiotics in the 30 days before CAR T infusion and evaluated for clinical characteristics and outcomes. The baseline and clinical characteristics of the patients in each of these group was compared to assess for variables that were associated with exposure to these antibiotics. Vital status is noted within 24 months of follow-up after CAR T cell infusion. Abbreviations: IQR: inter-quartile range; ALL: acute lymphoblastic leukemia; ECOG: Eastern Cooperative Oncology Group; CRS: cytokine release syndrome; ICANS: immune effector cell-associated neurotoxicity syndrome; No P-I-M antibiotic exposure: patients exposed to non-P-I-M plus patients who did not receive any antibiotics within the 4 weeks before CD19 CAR T cell infusion.

In addition, a higher abundance of Ruminococcus gnavus and amplicon sequence variant (ASV) 21 ([Ruminococcus] gnavus, species) was found to be associated with increased day 100 CR, and higher abundance of Bacteroides thetaiotaomicron species and ASV 29 (Bacteroides thetaiotaomicron) was associated with decreased CR at day 100 (FIG. 5N). Ruminococcus gnavus has been associated with T cell activation, including flares of Crohn's disease and increased lymphocyte counts following allogeneic hematopoietic cell transplantation. Of note, Ruminococcus gnavus produces an inflammatory complex polysaccharide that can mediate TNF-alpha secretion by dendritic cells. Conversely, Bacteroides thetaiotaomicron has been associated with anti-inflammatory effects, including attenuated severity of mouse colitis models, increased anti-inflammatory cytokine response with high TLR9 expression, increased differentiation of Treg/Th2 cells, and decreased development of Th1/Th17 cells. Taxa that were differentially associated with CAR-mediated toxicity using LEfSe were also identified. It was found that ASV 6 (Streptococcus salivarius) and ASV 253 (Oscillibacter ruminantium) were abundant in patients who experienced toxicity (FIG. 5O). In contrast, Lactobacillus salivarius species and ASV 6 (Lactobacillus salivarius) were abundant in patients who did not experience toxicity (FIG. 5O). Using LEfSe analysis of differentially abundant bacteria in the fecal samples from patients with Day 100 CR versus no CR an association between Ruminococcus gnavus (species) and Day 100 CR was found, as well as an association between Bacteroides thetaiotamicron (species) and no CR (FIG. 5P). These metagenomic analyses confirmed the bacterial taxa that were identified using 16S sequencing.

Possible mechanisms by which the gut microbiome can influence CD19 CAR T cell therapy response was explored via metagenomic shotgun sequencing of fecal samples. Using the metagenomics data, organism-specific gene hits were annotated according to the Kyoto Encyclopedia of Genes and Genomes Orthology. Based on these annotations, reads from each sample were reconstructed into metabolic pathways using the MetaCyc hierarchy of pathway classifications. Using LEfSe analysis, it was found that patients who had a Day 100 CR showed pathway enrichment for peptidoglycan biosynthesis (peptidoglycan biosynthesis IV Enterococcus faecium (PWY 6471)) (FIG. 5Q). In addition, it was found that patients who had a Day 100 CR showed pathway enrichment for aromatic amino acid biosynthesis (chorismate biosynthesis E. coli (ARO PWY), a superpathway of aromatic amino acid biosynthesis (COMPLETE ARO PWY)) and peptidoglycan biosynthesis V beta-lactam resistance (PWY 6470)) (FIG. 5Q). Of the aromatic amino acids, metabolites of tryptophan are agonists of the aryl hydrocarbon receptor (AhR), which is expressed by a number of immune cells and can modulate differentiation and function of T cells and dendritic cells. Activation of the Nod1 pathway by peptidoglycan fragments has been found to modulate the steady-state survival and turnover kinetics of circulating neutrophils and inflammatory monocytes—both of which are immune cells that have been implicated in the therapeutic and toxic response of CAR T cell therapy.

The LEfSe analysis of pathway enrichment based on toxicity was assessed (FIG. 5M). It was found that the non-oxidative branch of the pentose phosphate pathway (Nonoxipent PWY) is enriched in patients who experienced toxicity. This pathway produces D-erythrose 4-phosphate, a precursor of aromatic amino acids, and pyridoxal 5′-phosphate, the active form of pyridoxine. Pyridoxal 5′-phosphate serves several crucial roles, including acting as a cofactor for enzymes involved in tryptophan metabolism in bacteria as well as the host. Metagenomic shotgun sequencing of the baseline fecal samples demonstrates that metabolites produced by bacterial taxa can function as biomarkers of clinical outcomes in CD19 CAR T cell recipients.

### CONCLUSION

The Example discloses an association between the fecal microbiome composition and clinical outcomes of patients treated with CD19 CAR T cell immunotherapy. The Example shows that exposure to antibiotics and more specifically broad-spectrum antibiotics, such as piperacillin-tazobactam, imipenem-cilastatin, and meropenem (P-I-M), prior to CAR T cell infusion was associated with worse survival and increased toxicity in patients with B-cell malignancies. P-I-M exposure was associated with ICANS in the overall population, and in NHL but not in ALL. This finding could be related to the profoundly different nature of NHL compared to ALL. ALL is characterized by an aggressive proliferation with the leukemic cells being localized mostly in the bone marrow and blood, while NHL cells are usually found in lymphoid organs and are characterized by a profoundly altered environment. These differences might affect the indirect role of antibiotics on clinical outcomes. Overall, these data have potential implications on antibiotic stewardship in CAR T cell patients with B-cell malignancies.

It was also found that patients with NHL and ALL present with alteration in their fecal microbiome as noted by decreased alpha-diversity in comparison to healthy subjects before treatment with CAR T cells. Unbiased as well as hypothesis-driven taxonomic analysis of 16S sequencing of the fecal microbiome revealed that members within the class Clostridia are associated with Day 100 complete response. In particular, higher abundance of Ruminococcus, Bacteroides, and Faecalibacterium were associated with response to CD19 CAR T cell therapy.

The data on the relevance of the fecal microbiome to CD19 CAR T cell response aligns with prior cancer immunotherapy literature. In one study, members of Ruminococcus, specifically Ruminococcaceae, and Faecalibacterium were associated with better response to anti-PD1 immunotherapy. In this study, mice were treated with fecal microbiota transplant (FMT) from responders of anti-PD1 therapy and they were subsequently noted to have an enrichment of Faecalibacterium in the intestinal microbiome. The abundance of this genera was associated with increased intratumoral CD8+ T cells and innate effector cells (expressing CD45+CD11b+Ly6G+) as well as fewer splenic CD11b+CD11c+ expressing suppressive myeloid cells and splenic Fox3P+CD4+ Tregs. Higher abundances of Faecalibacterium and members of the genus Ruminococcus have also been associated with immune cell dynamics, including increased monocytes, neutrophils, and lymphocytes. Furthermore, several studies have found that Faecalibacterium prausnitzii is associated with improved response to immune checkpoint therapy. The mechanism whereby Faecalibacterium prausnitzii modulates the immune system is not well defined, but existing data suggests that it is associated with pro-inflammatory effects, including induction of TLR2 and TLR6. One of the metabolites produced by many bacteria within the phylum Firmicutes, including Faecalibacterium prausnitzii, is the short-chain fatty acid, butyrate. Data regarding the regulatory mechanisms of butyrate can aid in clarifying the seemingly paradoxical finding of increased abundance of Faecalibacterium prausnitzii associated with Day 100 CR and no toxicity based on the untargeted LEfSe analysis. Data supports that lower butyrate concentrations facilitate the differentiation of Tregs and increase the secretion of IL-10, while higher concentrations of butyrate induce expression T-bet and mediate IFN-gamma-producing Tregs or conventional T cells. Indeed, the activation of the Nod1 pathway by peptidoglycan fragments has been found to modulate the steady-state survival and turnover kinetics of circulating neutrophils and inflammatory monocytes—both of which are immune cells that have been implicated in the therapeutic and toxic response of CAR T cell therapy.

Of note, the boxplot distribution of the 16S bacterial taxa appears similar between responders and non-responders as well as between patients who did and did not experience toxicity. However, the Bayesian regression demonstrates increased odds of higher abundance of Ruminococcus associated with Day 100 CR. Without being limited to a particular theory, this difference can be due to the analytic tools used to assess this association. Whereas the Bayesian model is a multivariate regression analysis, the boxplot is a univariate analysis. The univariate analysis can mask relationships or otherwise not be able to detect marginal effects that can be assessed with multivariate analysis.

Overall, this study indicates a significant impact of the gut microbiota on CD19 CAR T cell therapy and a novel possibility to study therapeutic strategies that target the intestinal microbiome to improve clinical outcomes of patients treated with cellular therapies. The findings also support the hypothesis that the baseline microbial composition may correlate with efficacy and toxicity in CAR T cell recipients.

### REFERENCES

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

Patents, patent applications, publications, product descriptions and protocols are cited throughout, the disclosures of which are incorporated herein by reference in their entireties for all purposes.

What is claimed is:

1. A pharmaceutical composition comprising an effective amount of (a) a therapeutic bacterium or a spore thereof selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Streptococcus oralis, Lutispora thermophila, Agathobaculum butyriciproducens, Lactobacillus salivarius, Schaalia odontolytica, Longicatena caecimuris, Bifidobacterium breve, Ruminococcus bromii, Blautia luti, Faecalibacterium prausnitzii or a combination thereof; and/or (b) a bacterium or a spore thereof comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Streptococcus oralis, Lutispora thermophila, Agathobaculum butyriciproducens, Lactobacillus salivarius, Schaalia odontolytica, Longicatena caecimuris, Bifidobacterium breve, Ruminococcus bromii, Blautia luti, Faecalibacterium prausnitzii or a combination thereof.

2. The pharmaceutical composition of claim 1, further comprising a biocompatible pharmaceutical carrier.

3. The pharmaceutical composition of claim 1 or 2, wherein the pharmaceutical composition is formulated for oral, nasogastric, rectal, percutaneous or gastric tube administration.

4. The pharmaceutical composition of any one of claims 1-3, further comprising a probiotic bacteria, a probiotic yeast, a prebiotic, a postbiotic, an antibiotic or a combination thereof.

5. The pharmaceutical composition of any one of claims 1-4, wherein the pharmaceutical composition is in a form of a liquid, a suspension, a dried powder, a tablet, a capsule, a food product or a combination thereof.

6. The pharmaceutical composition of any one of claims 1-5, wherein the bacterium or spore thereof is a recombinant bacterium or a progeny thereof.

7. The pharmaceutical composition of any one of claims 1-6, comprising (a) a therapeutic bacterium or a spore thereof selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti, Faecalibacterium prausnitzii or a combination thereof; and/or (b) a bacterium or a spore thereof comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti, Faecalibacterium prausnitzii or a combination thereof.

8. The pharmaceutical composition of claim 7, comprising (a) a therapeutic bacterium or a spore thereof selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans or a combination thereof; and/or (b) a bacterium or a spore thereof comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans or a combination thereof.

9. The pharmaceutical composition of claim 7, comprising (a) a therapeutic bacterium or a spore thereof selected from the group consisting of Ruminococcus bromii, Longicatena caecimuris, Bifidobacterium breve, Blautia luti, Faecalibacterium prausnitzii or a combination thereof; and/or (b) a bacterium or a spore thereof comprising a 16S rRNA gene sequence having at least 94.5%, 95%, 97%, 98%, 98.7% or 99% sequence identity to the 16S rRNA gene sequence of Ruminococcus bromii, Longicatena caecimuris, Bifidobacterium breve, Blautia luti, Faecalibacterium prausnitzii or a combination thereof.

10. The pharmaceutical composition of any one of claims 1-6, comprising (a) a therapeutic bacterium or a spore thereof selected from the group consisting of Streptococcus oralis, Lutispora thermophila, Agathobaculum butyriciproducens, Lactobacillus salivarius, Schaalia odontolytica, Blautia luti, Faecalibacterium prausnitzii or a combination thereof; and/or (b) a bacterium or a spore thereof comprising a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica, Blautia luti, Faecalibacterium prausnitzii or a combination thereof.

11. The pharmaceutical composition of claim 10, comprising (a) a therapeutic bacterium or a spore thereof selected from the group consisting of Streptococcus oralis, Lutispora thermophila, Agathobaculum butyriciproducens, Lactobacillus salivarius, Schaalia odontolytica or a combination thereof; and/or (b) a bacterium or a spore thereof comprising a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica or a combination thereof.

12. The pharmaceutical composition of claim 10, comprising (a) a therapeutic bacterium or a spore thereof selected from the group consisting of Blautia luti, Faecalibacterium prausnitzii or a combination thereof; and/or (b) a bacterium or a spore thereof comprising a 16S rRNA gene sequence having at least 94.5%, 95%, 97%, 98%, 98.7% or 99% sequence identity to the 16S rRNA gene sequence of Blautia luti, Faecalibacterium prausnitzii or a combination thereof.

13. The pharmaceutical composition of any one of claims 7-9, wherein the effective amount of the bacterium or spore thereof increases the likelihood of cancer survival in a subject administered the pharmaceutical composition.

14. The pharmaceutical composition of any one of claims 10-12, wherein the effective amount of the bacterium or spore thereof decreases the likelihood of CAR T cell associated toxicity in a subject administered the pharmaceutical composition.

15. A method for identifying a subject having a cancer as having a decreased likelihood of cancer survival following a CAR T cell therapy comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a patient that did not exhibit cancer survival; and
(c) identifying the subject as having a decreased likelihood of cancer survival if the level of the diagnostic bacterium or spore thereof is higher than the reference level;
wherein the bacterium or spore thereof is selected from the group consisting of Hungatella effluvii, Coprobacillus cateniformis, Atopobium parvulum, Faecalicoccus acidiformans, Lactococcus lactis, Anaerobacterium chartisolvens, Atopobiaceae Clostridium innocuum, Bacteroides thetaiotaomicron, a species of the Veillonellaceae family and a combination thereof; or the bacterium or spore thereof comprises a 16S rRNA gene sequence having at least 94.5%, 95%, 97%, 98%, 98.7% or 99% sequence identity to the 16S rRNA gene sequence of Hungatella effluvii, Coprobacillus cateniformis, Atopobium parvulum, Faecalicoccus acidiformans, Lactococcus lactis, Anaerobacterium chartisolvens, Atopobiaceae Clostridium innocuum, Bacteroides thetaiotaomicron, a species of the Veillonellaceae family or a combination thereof.

16. The method of claim 15, wherein the bacterium or spore thereof is selected from the group consisting of Hungatella effluvii, Coprobacillus cateniformis, Atopobium parvulum, Faecalicoccus acidiformans, Lactococcus lactis, Anaerobacterium chartisolvens, Atopobiaceae Clostridium innocuum, Bacteroides thetaiotaomicron and a combination thereof; or the bacterium or spore thereof comprises a 16S rRNA gene sequence having at least 94.5%, 95%, 97%, 98%, 98.7% or 99% sequence identity to the 16S rRNA gene sequence of Hungatella effluvii, Coprobacillus cateniformis, Atopobium parvulum, Faecalicoccus acidiformans, Lactococcus lactis, Anaerobacterium chartisolvens, Atopobiaceae Clostridium innocuum, Bacteroides thetaiotaomicron or a combination thereof.

17. The method of claim 15, wherein the bacterium or spore thereof is a species of the Veillonellaceae family or is a bacterium or spore thereof comprising a 16S rRNA gene sequence having at least 94.5%, 95%, 97%, 98%, 98.7% or 99% sequence identity to the 16S rRNA gene sequence of a species of the Veillonellaceae family.

18. The method of any one of claims 15-17, further comprising treating the subject identified as having a decreased likelihood of exhibiting cancer survival with the pharmaceutical composition of any one of claims 1-9.

19. A method for identifying a subject having a cancer as having an increased likelihood of cancer survival following a CAR T cell therapy comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a patient exhibited cancer survival; and
(c) identifying the subject as having increased likelihood of cancer survival if the level of the diagnostic bacterium or spore thereof is higher than the reference level;
wherein the bacterium or spore thereof is selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Longicatena caecimuris, Bifidobacterium breve, Escherichia coli, Clostridium methoxybenzovorans, Ruminococcus bromii, Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Longicatena caecimuris, Bifidobacterium breve, Escherichia coli, Clostridium methoxybenzovorans, Ruminococcus bromii, Blautia luti, Faecalibacterium prausnitzii or a combination thereof.

20. The method of claim 19, wherein the bacterium or spore thereof is selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans and a combination thereof; or the bacterium or spore thereof comprises a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans or a combination thereof.

21. The method of claim 19, wherein the bacterium or spore thereof is selected from the group consisting of Longicatena caecimuris, Bifidobacterium breve, Escherichia coli, Ruminococcus bromii, Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Longicatena caecimuris, Bifidobacterium breve, Escherichia coli, Ruminococcus bromii, Blautia luti, Faecalibacterium prausnitzii or a combination thereof.

22. A method for identifying a subject having a cancer as having an increased likelihood of cancer survival comprising:
(a) determining a level of a bacterial gene in a sample of the subject;
(b) comparing the level of the bacterial gene to a reference bacterial gene level, wherein the reference level is the level of the bacterial gene in a patient that exhibited cancer survival; and
(c) identifying the subject as having an increased likelihood of cancer survival if the level of the bacterial gene is higher than the reference bacterial gene level;
wherein the bacterial gene is a gene involved in aromatic amino acid biosynthesis and/or peptidoglycan biosynthesis.

23. The method of any one of claims 19-22, further comprising treating the subject with a CAR T cell therapy.

24. The method of any one of claims 19-23, wherein cancer survival is the survival of the subject at least about 100 days following a CAR T cell therapy.

25. A method for identifying a subject having a cancer as having an increased likelihood of exhibiting a CAR T cell associated toxicity comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a patient that exhibited a CAR T cell associated toxicity; and
(c) identifying the subject as having an increased likelihood of exhibiting a CAR T cell associated toxicity if the level of the bacterium or spore thereof is higher than the reference level;
wherein the bacterium or spore thereof is selected from the group consisting of Streptococcus salivarius, Oscillibacter ruminantium, Eubacterium ramulus, Streptococcus gordonii, Caprocicproducens galactitolivorans, Clostridium hylemonae and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus salivarius, Oscillibacter ruminantium, Eubacterium ramulus, Streptococcus gordonii, Caprocicproducens galactitolivorans, Clostridium hylemonae and a combination thereof.

26. The method of claim 25, further comprising treating the subject identified as having an increased likelihood of exhibiting a CAR T cell associated toxicity with the pharmaceutical composition of any one of claims 1-6 and 10-12.

27. The method of claim 25 or 26, further comprising treating the subject with a CAR T cell therapy.

28. A method for identifying a subject having a cancer as having a decreased likelihood of exhibiting a CAR T cell associated toxicity comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a patient that did not exhibit a CAR T cell associated toxicity; and
(c) identifying the subject as having a decreased likelihood of exhibiting a CAR T cell associated toxicity if the level of the bacterium or spore thereof is higher than the reference level;
wherein the bacterium or spore thereof is selected from the group consisting of Streptococcus oralis, Lutispora thermophila, Agathobaculum butyriciproducens, Lactobacillus salivarius, Schaalia odontolytica, Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica, Blautia luti, Faecalibacterium prausnitzii and a combination thereof.

29. The method of claim 28, wherein the bacterium or spore thereof is selected from the group consisting of Streptococcus oralis, Lutispora thermophila, Agathobaculum butyriciproducens, Lactobacillus salivarius, Schaalia odontolytica and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica and a combination thereof.

30. The method of claim 28, wherein the bacterium or spore thereof is selected from the group consisting of Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Blautia luti, Faecalibacterium prausnitzii and a combination thereof.

31. A method for identifying a subject having a cancer as having a decreased likelihood to exhibit cancer survival and/or an increased likelihood to exhibit a CAR T cell associated toxicity comprising:
(a) determining if the subject has been administered an antibiotic; and
(b) identifying the subject as having an increased likelihood of a CAR T cell associated toxicity and/or a decreased likelihood of cancer survival if the subject has been administered piperacillin-tazobactam, imipenem-cilastatin, meropenem or a combination thereof.

32. The method of claim 31, wherein the antibiotic is administered less than about 4 weeks prior to the initiation of a CAR T cell therapy.

33. The method of any one of claims 25-32, wherein the CAR T cell associated toxicity is cytokine release syndrome (CRS) and/or immune effector cell-associated neurotoxicity syndrome (ICANS).

34. A method for treating a subject having a cancer with a CAR T cell therapy, comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a subject that exhibited cancer survival;
(c) identifying the subject as having an increased likelihood of cancer survival if the level of the bacterium or spore thereof is higher than the reference level; and
(d) treating the subject identified as having an increased likelihood of cancer survival with a CAR T cell therapy,
wherein the bacterium or spore thereof is selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Blautia luti, Longicatena caecimuris, Bifidobacterium breve, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Blautia luti, Longicatena caecimuris, Bifidobacterium breve or Faecalibacterium prausnitzii.

35. The method of claim 34, wherein the bacterium or spore thereof is selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans and a combination thereof; or the bacterium or spore thereof comprises a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis or Clostridium methoxybenzovorans.

36. The method of claim 34, wherein the bacterium or spore thereof is selected from the group consisting of Ruminococcus bromii, Blautia luti, Longicatena caecimuris, Bifidobacterium breve, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Ruminococcus bromii, Blautia luti, Longicatena caecimuris, Bifidobacterium breve or Faecalibacterium prausnitzii.

37. A method for treating a subject having a cancer with a CAR T cell therapy, comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a subject that did not exhibit cancer survival;
(c) identifying the subject as having a decreased likelihood of cancer survival if the level of the bacterium or spore thereof is higher than the reference level; and
(d) treating the subject identified as having a decreased likelihood of cancer survival with a pharmaceutical composition comprising one or more therapeutic bacteria and a CAR T cell therapy,
wherein the bacterium or spore thereof is selected from the group consisting of Hungatella effluvii, Coprobacillus cateniformis, Atopobium parvulum, Faecalicoccus acidiformans, Lactococcus lactis, Anaerobacterium chartisolvens, Clostridium innocuum, Bacteroides thetaiotaomicron, a species of the Veillonellaceae family and a combination thereof; or the bacterium or spore thereof comprises a 16S rRNA gene sequence having at least 94.5%, 95%, 97%, 98%, 98.7% or 99% sequence identity to the 16S rRNA gene sequence of Hungatella effluvii, Coprobacillus cateniformis, Atopobium parvulum, Faecalicoccus acidiformans, Lactococcus lactis, Anaerobacterium chartisolvens, Clostridium innocuum, Bacteroides thetaiotaomicron, a species of the Veillonellaceae family and a combination thereof,
wherein the therapeutic bacteria comprises one or more of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti and/or Faecalibacterium prausnitzii or a bacteria comprising a 16S rRNA gene sequence having at least 94.5%, 95%, 97%, 98%, 98.7% or 99% sequence identity to the 16S rRNA gene sequence of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Ruminococcus bromii, Longicatena caecimuris, Bifidobacterium breve, Blautia luti and/or Faecalibacterium prausnitzii.

38. The method of any one of claims 34-37, wherein cancer survival is the survival of the subject at least about 100 day following a CAR T cell therapy.

39. A method for treating a subject having a cancer with a CAR T cell therapy, comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a subject that did not exhibit a CAR T cell associated toxicity;
(c) identifying the subject as having a decreased likelihood of exhibiting a CAR T cell associated toxicity if the level of the bacterium or spore thereof is higher than the reference level; and
(d) treating the subject identified as having a decreased likelihood of exhibiting a CAR T cell associated toxicity with a CAR T cell therapy,
wherein the bacterium or spore thereof is selected from the group consisting of Streptococcus oralis, Lutispora thermophila, Agathobaculum butyriciproducens, Lactobacillus salivarius, Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Blautia luti and/or Faecalibacterium prausnitzii.

40. The method of claim 39, wherein the bacterium or spore thereof is selected from the group consisting of Streptococcus oralis, Lutispora thermophila, Agathobaculum butyriciproducens, Lactobacillus salivarius and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens and/or Lactobacillus salivarius.

41. The method of claim 39, wherein the bacterium or spore thereof is selected from the group consisting of Blautia luti, Faecalibacterium prausnitzii and a combination thereof; or the bacterium or spore thereof comprises a 16S rRNA gene sequence having at least 94.5%, 95%, 97%, 98%, 98.7% or 99% sequence identity to the 16S rRNA gene sequence of Blautia luti and/or Faecalibacterium prausnitzii.

42. A method for treating a subject having a cancer with a CAR T cell therapy, comprising:
(a) determining a level of a bacterium or a spore thereof in a sample of the subject;
(b) comparing the level of the bacterium or spores thereof to a reference level of the bacterium or a spore thereof, wherein the reference level is the level of the bacterium or a spore thereof in a subject that exhibited CAR T cell associated toxicity;
(c) identifying the subject as having an increased likelihood of exhibiting a CAR T cell associated toxicity if the level of the bacterium or spore thereof is higher than the reference level; and
(d) treating the subject identified as having an increased likelihood of exhibiting a CAR T cell associated toxicity with a pharmaceutical composition comprising one or more therapeutic bacteria and a CAR T cell therapy,
wherein the bacterium or spore thereof is selected from the group consisting of Streptococcus salivarius, Oscillibacter ruminantium, Eubacterium ramulus, Streptococcus gordonii, Caprocicproducens galactitolivorans, Clostridium hylemonae and a combination thereof; or the bacterium or spore thereof comprises a 16S rRNA gene sequence having at least 94.5%, 95%, 97%, 98%, 98.7% or 99% sequence identity to the 16S rRNA gene sequence of Streptococcus salivarius, Oscillibacter ruminantium, Eubacterium ramulus, Streptococcus gordonii, Caprocicproducens galactitolivorans, Clostridium hylemonae or a combination thereof,
wherein the therapeutic bacteria comprises one or more of Streptococcus oralis, Lutispora thermophila, Agathobaculum butyriciproducens, Lactobacillus salivarius, Schaalia odontolytica, Blautia luti, Faecalibacterium prausnitzii or a bacterium comprising a 16S rRNA gene sequence having at least 94.5%, 95%, 97%, 98%, 98.7% or 99% sequence identity to the 16S rRNA gene sequence of Streptococcus oralis, Lutispora thermophila, Agathobaculum butyriciproducens, Lactobacillus salivarius, Schaalia odontolytica, Blautia luti, Faecalibacterium prausnitzii or a combination thereof.

43. The method of any one of claims 38-42, wherein the CAR T cell associated toxicity is cytokine release syndrome (CRS) and/or immune effector cell-associated neurotoxicity syndrome (ICANS).

44. The method of any one of claims 15-43, wherein the sample is a fecal sample or an intestinal content sample of the subject.

45. The method of any one of claims 15-44, further comprising administering to the subject a chemotherapy, immunotherapy, stem cell therapy, cellular therapy, a probiotic bacteria, a probiotic yeast, a prebiotic, a postbiotic, an antibiotic or a combination thereof.

46. The method of any one of claims 15-45, wherein the cancer is selected from the group consisting of acute lymphoblastic leukemia (ALL), chronic lymphoblastic leukemia (CLL) or non-Hodgkin lymphoma (NHL)).

47. The method of any one of claims 15-46, wherein the CAR T cell therapy comprises a CAR T cell that targets mucin 16 (MUC16), B-cell maturation antigen (BCMA) and/or CD19.

48. A kit comprising the pharmaceutical composition of any one of claims 1-14.

49. A kit comprising means for identifying a bacterium or a spore thereof selected from the group consisting of Ruminococcus gnavus, Blautia schinkii, Bacteroides finegoldii, Clostridium glycyrrhizinilyticum, Clostridium saccharolyticum, Clostridium celerecrescens, Anaeromassilibacillus senegalensis, Clostridium methoxybenzovorans, Hungatella effluvii, Coprobacillus cateniformis, Atopobium parvulum, Faecalicoccus acidiformans, Lactococcus lactis, Anaerobacterium chartisolvens, Atopobiaceae, Clostridium innocuum, Bacteroides thetaiotamicron, Streptococcus salivarius, Oscillibacter ruminantium, Eubacterium ramulus, Streptococcus gordonii, Caprocicproducens galactitolivorans, Clostridium hylemonae, Streptococcus oralis, Lutispora thermophila, Agathobaculum butyriciproducens, Schaalia odontolytica, Lactobacillus salivarius, Ruminococcus bromii, Bifidobacterium breve, Longicatena caecimuris, Escherichia coli, Blautia luti, Faecalibacterium prausnitzii and a combination thereof.

50. The kit of claim 48 or 49, further comprising instructions for treating a subject having a cancer.

51. The kit of any one of claims 48-50, further comprising instructions for identifying the subject as having an increased likelihood or decreased likelihood of exhibiting cancer survival, wherein the instructions comprise:
(a) determining the level of the bacterium or spore thereof in a sample of the subject;
(b) comparing the level of the bacterium or spores thereof to a reference level of the bacterium or a spore thereof; and
(c) identifying the subject as having an increased likelihood or decreased likelihood of exhibiting cancer survival based on the comparison.

52. The kit of any one of claims 48-51, further comprising instructions for identifying the subject as having an increased likelihood or decreased likelihood of a CAR T cell associated toxicity, wherein the instructions comprise:
(a) determining the level of the bacterium or spore thereof in a sample of the subject;
(b) comparing the level of the bacterium or spores thereof to a reference level of the bacterium or a spore thereof; and
(c) identifying the subject having an increased likelihood or decreased likelihood of a CAR T cell associated toxicity based on the comparison.

53. Use of a pharmaceutical composition of any one of claims 1-14 for treating a subject having a cancer.

54. The use of claim 53, wherein the cancer is being treated or will be treated with a CAR T cell therapy.

55. Use of a pharmaceutical composition of any one of claims 7-9 for increasing the likelihood of cancer survival in a subject.

56. The use of claim 55, wherein the subject is being treated or will be treated with a CAR T cell therapy.

57. Use of a pharmaceutical composition of any one of claims 10-12 for decreasing the likelihood of a CAR T cell associated toxicity in a subject.

58. The use of claim 57, wherein the subject is being treated or will be treated with a CAR T cell therapy.
